(19) United States
(12) Patent Application Publication
(10) Pub. No.: US 2011/0196614 A1
(43) Pub. Date: Aug. 11, 2011

(54) BLOOD TRANSCRIPTIONAL SIGNATURE OF MYCOBACTERIUM TUBERCULOSIS INFECTION

(75) Inventors: Jacques F. Banchereau, Dallas, TX (US); Damien Chaussabel, Bainbridge Island, WA (US); Anne O'Garra, London (GB); Matthew Berry, London (GB); Onn Min Kon, London (GB)

(73) Assignees: BAYLOR RESEARCH INSTITUTE, Dallas, TX (US); NATIONAL INSTITUTE FOR MEDICAL RESEARCH, London (GB); IMPERIAL COLLEGE HEALTHCARE NHS TRUST, London (GB)

(21) Appl. No.: 12/602,488

(22) PCT Filed: Jun. 25, 2009
(86) PCT No.: PCT/US09/48698

§ 371 Date: Apr. 25, 2011

Related U.S. Application Data
(60) Provisional application No. 61/075,728, filed on Jun. 25, 2008.

(51) Int. Cl. G06F 19/00; G01N 33/48

(52) U.S. Cl. 702/19

(57) ABSTRACT

The present invention includes methods, systems and kits for distinguishing between active and latent mycobacterium tuberculosis infection in a patient suspected of being infected with mycobacterium tuberculosis, and distinguishing such patients from uninfected individuals, the method including the steps of obtaining a gene expression dataset from a whole blood obtained sample from the patient and determining the differential expression of one or more transcriptional gene expression modules that distinguish between infected and non-infected patients, wherein the dataset demonstrates an aggregate change in the levels of polynucleotides in the one or more transcriptional gene expression modules as compared to matched non-infected patients, thereby distinguishing between active and latent mycobacterium tuberculosis infection.

### TECHNICAL FIELD OF THE INVENTION

The present invention relates in general to the field of Mycobacterium tuberculosis infection, and more particularly, to a system, method and apparatus for the diagnosis, prognosis and monitoring of latent and active Mycobacterium tuberculosis infection and disease progression before, during and after treatment.

### LENGTHY TABLE

The patent application contains a lengthy table section. A copy of the table is available in electronic form from the USPTO web site (http://seqdata.uspto.gov/). An electronic copy of the table will also be available from the USPTO upon request and payment of the fee set forth in 37 CFR 1.19(b)(3).

### BACKGROUND OF THE INVENTION

Without limiting the scope of the invention, its background is described in connection with the identification and treatment of Mycobacterium tuberculosis infection.

Pulmonary tuberculosis (PTB) is a major and increasing cause of morbidity and mortality worldwide caused by Mycobacterium tuberculosis (M. tuberculosis). However, the majority of individuals infected with M. tuberculosis remain asymptomatic, retaining the infection in a latent form and it is thought that this latent state is maintained by an active immune response (WHO; Kaufmann, S H & McMichael, A J., Nat Med, 2005). This is supported by reports showing that treatment of patients with Crohn's Disease or Rheumatoid Arthritis with anti-TNF antibodies, results in improvement of autoimmune symptoms, but on the other hand causes reactivation of TB in patients previously in contact with M. tuberculosis (Keane). The immune response to M. tuberculosis is multifactorial and includes genetically determined host factors, such as TNF, and IFN-γ and IL-12, of the Th1 axis (Reviewed in Casanova, Ann Rev; Newport). However, immune cells from adult pulmonary TB patients can produce IFN-γ, IL-12 and TNF, and IFN-γ therapy does not help to ameliorate disease (Reviewed in Reljic, 2007, J Interferon & Cyt Res., 27, 353-63), suggesting that a broader number of host immune factors are involved in protection against M. tuberculosis and the maintenance of latency. Thus, a knowledge of host factors induced in latent versus active TB may provide information with respect to the immune response which can control infection with M. tuberculosis.

The diagnosis of PTB can be difficult and problematic for a number of reasons. Firstly demonstrating the presence of typical M. tuberculosis bacilli in the sputum by microscopy examination (smear positive) has a sensitivity of only 50-70%, and positive diagnosis requires isolation of M. tuberculosis by culture, which can take up to 8 weeks. In addition, some patients are smear negative on sputum or are unable to produce sputum, and thus additional sampling is required by bronchoscopy, an invasive procedure. Due to these limitations in the diagnosis of PTB, smear negative patients are sometimes tested for tuberculin (PPD) skin reactivity (Mantoux). However, tuberculin (PPD) skin reactivity cannot distinguish between BCG vaccination, latent or active TB. In response to this problem, assays have been developed demonstrating immunoreactivity to specific M. tuberculosis antigens, which are absent in BCG. Reactivity to these M. tuberculosis antigens, as measured by production of IFN-γ by blood cells in Interferon Gamma Release Assays (IGRA), however, does not differentiate latent from active disease. Latent TB is defined in the clinic by a delayed type hypersensitivity reaction when the patient is intradermally challenged with PPD, together with an IGRA positive result, in the absence of clinical symptoms or signs, or radiology suggestive of active disease. The reactivation of latent/dormant tuberculosis (TB) presents a major health hazard with the risk of transmission to other individuals, and thus biomarkers reflecting differences in latent and active TB patients would be of use in disease management, particularly since anti-mycobacterial drug treatment is arduous and can result in serious side-effects.

### SUMMARY OF THE INVENTION

The present invention includes methods and kits for the identification of latent versus active tuberculosis (TB) patients, as compared to healthy controls. In one embodiment, microarray analysis of blood of a distinct and reciprocal immune signature is used to determine, diagnose, track and treat latent versus active tuberculosis (TB) patients.

In one embodiment, the present invention includes methods, systems and kits for distinguishing between active and latent Mycobacterium tuberculosis infection in a patient suspected of being infected with Mycobacterium tuberculosis, the method including the steps of: obtaining a gene expression dataset from a whole blood sample from the patient; determining the differential expression of one or more transcriptional gene expression modules that distinguish between infected patients and non-infected individuals, wherein the dataset demonstrates an aggregate change in the levels of polynucleotides in the one or more transcriptional gene expression modules as compared to matched non-infected individuals, and distinguishing between active and latent Mycobacterium tuberculosis (TB) infection based on the one or more transcriptional gene expression modules that differentiate between active and latent infection. In one aspect, the invention may also include the step of using the determined comparative gene product information to formulate a diagnosis.

In another aspect, the method may also include the step of using the determined comparative gene product information to formulate a prognosis or the step of using the determined comparative gene product information to formulate a treatment plan. In one alternative aspect, the method may include the step of distinguishing patients with latent TB from active TB patients. In one aspect, the module may include a dataset of the genes in modules M1.2, M1.3, M1.4, M1.5, M1.8, M2.1, M2.4, M2.8, M3.1, M3.2, M3.3, M3.4, M3.6, M3.7, M3.8 or M3.9 to detect active pulmonary infection. In another aspect, the module may include a dataset of the genes in modules M1.5, M2.1, M2.6, M2.10, M3.2 or M3.3 to detect a latent infection. In yet another aspect, the following genes are down-regulated in active pulmonary infection CD3, CTLA-4, CD28, ZAP-70, IL-7R, CD2, SLAM, CCR7 and GATA-3. In one specific aspect, the expression profile of the modules in FIG. 9 is indicative of active pulmonary infection and the expression profile of the modules in FIG. 10 is indicative of latent infection. It has been found that the underexpression of genes in modules M3.4, M3.6, M3.7, M3.8 and M3.9 is indicative of active infection. It has also been found that the overexpression of genes in modules M3.1 is indicative of active infection.

In yet another aspect of the present invention, the method may also include the step of distinguishing TB infection from other bacterial infections by determining the gene expression in modules M2.2, M2.3 and M3.5, which are overexpressed by the peripheral blood mononuclear cells or whole blood in infection other than Mycobacterium. Alternatively, the method may include the step of distinguishing the differential and reciprocal transcriptional signatures in the blood of latent and active TB patients using two or more of the following modules: M1.3, M1.4, M1.5, M1.8, M2.1, M2.4, M2.8, M3.1, M3.2, M3.3, M3.4, M3.6, M3.7, M3.8 or M3.9 for active pulmonary infection and modules M1.5, M2.1, M2.6, M2.10, M3.2 or M3.3 for a latent infection. Examples of the genes that are upregulated in active pulmonary TB infection versus a healthy patient are selected from Tables 7A, 7D, 71, 7J and 7K. Further examples of the genes that are downregulated in active pulmonary TB infection versus a healthy patient are selected from Tables 7B, 7C, 7E, 7F, 7G, 7H, 7L, 7M, 7N, 70 and 7P. In one specific aspect, the genes that are upregulated in latent TB infection versus a healthy patient may be selected from Table 8B. In another specific aspect, the genes that are downregulated in latent TB infection versus a healthy patient may be selected from Tables 8A, 8C, 8D, 8E and 8F.

Another embodiment of the present invention is a method for distinguishing between active and latent Mycobacterium tuberculosis infection in a patient suspected of being infected with Mycobacterium tuberculosis, the method including the steps of: obtaining a first gene expression dataset obtained from a first clinical group with active Mycobacterium tuberculosis infection, a second gene expression dataset obtained from a second clinical group with a latent Mycobacterium tuberculosis infection patient and a third gene expression dataset obtained from a clinical group of non-infected individuals; generating a gene cluster dataset comprising the differential expression of genes between any two of the first, second and third datasets; and determining a unique pattern of expression/representation that is indicative of latent infection, active infection or being healthy. In one aspect, each clinical group is separated into a unique pattern of expression/representation for each of the 119 genes of Table 6. In another aspect, values for the first and third datasets are compared and the values for the dataset from the third dataset are subtracted therefrom. In another specific aspect, the values for the second and third datasets are compared and the values for the dataset from the third dataset are subtracted therefrom. In one specific embodiment, the method may further include the step of comparing values for two different datasets and subtracting the values for the remaining dataset to distinguish between a patient with a latent infection, a patient with an active infection and a non-infected individual. In one aspect, the method may further comprise the step of using the determined comparative gene product information to formulate a diagnosis or a prognosis. In yet another aspect, the method includes the step of using the determined comparative gene product information to formulate a treatment plan. The method may also include the step of distinguishing patients with latent TB from active TB patients by analyzing the expression/representation of genes in the gene and patient clusters.

In one specific aspect, the method may further include the step of determining the expression levels of the genes: ST3GAL6, PAD14, TNFRSF12A, VAMP3, BR13, RGS19, PILRA, NCF1, LOC652616, PLAUR(CD87), SIGLEC5, B3GALT7, IBRDC3(NKLAM), ALOX5AP(FLAP), MMP9, ANPEP(APN), NALP12, CSF2RA, IL6R(CD126), RASGRP4, TNFSF14(CD258), NCF4, HK2, ARID3A, PGLYRP1(PGRP), which are underexpressed/underrepresented in the blood of Latent TB patients but not in the blood of Healthy individuals or Active TB patients. In another specific aspect, the method may further include the step of determining the expression levels of the genes: ABCG1, SREBF1, RBP7(CRBP4), C22orf5, FAM101B, S100P, LOC649377, UBTD1, PSTPIP-1, RENBP, PGM2, SULF2, FAM7A1, HOM-TES-103, NDUFAF1, CES1, CYP27A1, FLJ33641, GPR177, MID1 IP1(MIG-12), PSD4, SF3A1, NOV(CCN3), SGK(SGK1), CDK5R1, LOC642035, which are overexpressed/overrepresented in the blood of Healthy control individuals but were underexpressed/underrepresented in the blood of Latent TB patients, and underexpressed/underrepresented in the blood of Active TB patients. In another specific aspect, the method may further include the step of determining the expression levels of the genes: ARSG, LOC284757, MDM4, CRNKL1, IL8, LOC389541, CD300LB, NIN, PHKG2, HIP1, which are overexpressed/overrepresented in the blood of Healthy individuals, are underexpressed/underrepresented in the blood of both Latent and Active TB patients. In one specific aspect, the method may further include the step of determining the expression levels of the genes: PSMB8(LMP7), APOL6, GBP2, GBP5, GBP4, ATF3, GCH1, VAMPS, WARS, LIMK1, NPC2, IL-15, LMTK2, STX11(FHL4), which are overexpressed/overrepresented in the blood of Active TB, and underexpressed/underrepresented in the blood of Latent TB patients and Healthy control individuals. In one specific aspect, the method may further include the step of determining the expression levels of the genes: FLJ11259(DRAM), JAK2, GSDMDC1(DF5L)(FKSG10), SIPAIL1, [2680400](KIAA1632), ACTA2(ACTSA), KCNMB1(SLO-BETA), which are overexpressed/overrepresented in blood from Active TB patients, and underexpressed/underrepresented in the blood from Latent TB patients and Healthy control individuals. In one specific aspect, the method may further include the step of determining the expression levels of the genes: SPTANI, KIAAD179(Nnp1)(RRP1), FAM84B(NSE2), SELM, IL27RA, MRPS34, [6940246](IL23A), PRKCA(PKCA), CCDC41, CD52(CDW52), [3890241](ZN404), MCCC1(MCCA/B), SOX8, SYNJ2, FLJ21127, FHIT, which are underexpressed/underrepresented in the blood of Active TB patients but not in the blood of Latent TB patients or Healthy Control individuals. In one specific aspect, the method may further include the step of determining the expression levels of the genes: CDKL1(p42), MICALCL, MBNL3, RHD, ST7(RAY1), PPR3R1, [360739](PIP5K2A), AMFR, FLJ22471, CRAT(CAT1), PLA2G4C, ACOT7(ACT)(ACH1), RNF182, KLRC3(NKG2E), HLA-DPB1, which are underexpressed/underrepresented in the blood of Healthy Control individuals, overexpressed/overrepresented in the blood of the Latent TB patients, and overexpressed/overrepresented in the blood of Active TB patients.

Yet another embodiment of the present invention is a method for distinguishing between active and latent mycobacterium tuberculosis infection in a patient suspected of being infected with Mycobacterium tuberculosis, the method including the steps of: obtaining a gene expression dataset from a whole blood sample; sorting the gene expression dataset into one or more transcriptional gene expression modules; and mapping the differential expression of the one or more transcriptional gene expression modules that distinguish between active and latent Mycobacterium tuberculosis infection, thereby distinguishing between active and latent Mycobacterium tuberculosis infection. In one aspect, the dataset includes TRIM genes. In one aspect, the dataset includes TRIM genes, specifically, TRIM 5, 6, 19(PML), 21, 22, 25, 68 are overrepresented/expressed in active pulmonary TB. In one aspect, the dataset of TRIM genes, includes TRIM 28, 32, 51, 52, 68, are underepresented/expressed in active pulmonary TB.

Another embodiment of the present invention is a method of diagnosing a patient with active and latent Mycobacterium tuberculosis infection in a patient suspected of being infected with mycobacterium tuberculosis, the method comprising detecting differential expression of one or more transcriptional gene expression modules that distinguish between infected and non-infected patients obtained from whole blood, wherein whole blood demonstrates an aggregate change in the levels of polynucleotides in the one or more transcriptional gene expression modules as compared to matched non-infected patients, thereby distinguishing between active and latent mycobacterium tuberculosis infection. In another aspect, the method includes one or more of the step of: using the determined comparative gene product information to formulate a diagnosis, the step of using the determined comparative gene product information to formulate a prognosis and the step of using the determined comparative gene product information to formulate a treatment plan. In one alternative aspect, the method may include the step of distinguishing patients with latent TB from active TB patients. In one aspect, the module may include a dataset of the genes in modules M1.2, M1.3, M1.4, M1.5, M1.8, M2.1, M2.4, M2.8, M3.1, M3.2, M3.3, M3.4, M3.6, M3.7, M3.8 or M3.9 to detect active pulmonary infection. In another aspect, the module may include a dataset of the genes in modules M1.5, M2.1, M2.6, M2.10, M3.2 or M3.3 to detect a latent infection. In yet another aspect, the following genes are down-regulated in active pulmonary infection CD3, CTLA-4, CD28, ZAP-70, IL-7R, CD2, SLAM, CCR7 and GATA-3. In one specific aspect, the expression profile of the modules in FIG. 9 is indicative of active pulmonary infection and the expression profile of the modules in FIG. 10 is indicative of latent infection. It has been found that the underexpression of genes in modules M3.4, M3.6, M3.7, M3.8 and M3.9 is indicative of active infection. It has also been found that the overexpression of genes in modules M3.1 is indicative of active infection.

In yet another aspect of the present invention, the method may also include the step of distinguishing TB infection from other bacterial infections by determining the gene expression in modules M2.2, M2.3 and M3.5, which are overexpressed by the peripheral blood mononuclear cells or whole blood in infection other than Mycobacterium. Alternatively, the method may include the step of distinguishing the differential and reciprocal transcriptional signatures in the blood of latent and active TB patients using two or more of the following modules: M1.3, M1.4, M1.5, M1.8, M2.1, M2.4, M2.8, M3.1, M3.2, M3.3, M3.4, M3.6, M3.7, M3.8 or M3.9 for active pulmonary infection and modules M1.5, M2.1, M2.6, M2.10, M3.2 or M3.3 for a latent infection. Examples of the genes that are upregulated in active pulmonary TB infection versus a healthy patient are selected from Tables 7A, 7D, 71, 7J and 7K. Further examples of the genes that are downregulated in active pulmonary TB infection versus a healthy patient are selected from Tables 7B, 7C, 7E, 7F, 7G, 7H, 7L, 7M, 7N, 7O and 7P. In one specific aspect, the genes that are upregulated in latent TB infection versus a healthy patient may be selected from Table 8B. In another specific aspect, the genes that are downregulated in latent TB infection versus a healthy patient may be selected from Tables 8A, 8C, 8D, 8E and 8F.

Another embodiment of the present invention is a kit for diagnosing a patient with active and latent mycobacterium tuberculosis infection in a patient suspected of being infected with Mycobacterium tuberculosis, the kit that includes a gene expression detector for obtaining a gene expression dataset from the patient; and a processor capable of comparing the gene expression to pre-defined gene module dataset that distinguish between infected and non-infected patients obtained from whole blood, wherein whole blood demonstrates an aggregate change in the levels of polynucleotides in the one or more transcriptional gene expression modules as compared to matched non-infected patients, thereby distinguishing between active and latent Mycobacterium tuberculosis infection.

Yet another embodiment includes a system of diagnosing a patient with active and latent Mycobacterium tuberculosis infection comprising: a gene expression dataset from the patient; and a processor capable of comparing the gene expression to pre-defined gene module dataset that distinguish between infected and non-infected patients obtained from whole blood, wherein whole blood demonstrates an aggregate change in the levels of polynucleotides in the one or more transcriptional gene expression modules as compared to matched non-infected patients, thereby distinguishing between active and latent Mycobacterium tuberculosis infection, wherein the modules are selected from M1.3, M1.4, M1.5, M1.8, M2.1, M2.4, M2.8, M3.1, M3.2, M3.3, M3.4, M3.6, M3.7, M3.8 or M3.9 for active pulmonary infection and modules M1.5, M2.1, M2.6, M2.10, M3.2 or M3.3 for a latent infection.

### BRIEF DESCRIPTION OF THE DRAWINGS

For a more complete understanding of the features and advantages of the present invention, reference is now made to the detailed description of the invention along with the accompanying figures and in which:

FIG. 1 shows the gene array expression results from 42 participants, genes present in at least 2 samples (PAL2), genes 2 folds over or under represented compared with median, clustered by Pearson Correlation comparing active PTB, latent TB, healthy BCG non-vaccinated controls and healthy BCG vaccinated controls;

FIG. 2 shows the gene array expression results from PAL2, 2 folds up or down expressed, filtered for statistically significant differences in expression between clinical groups using a non-parametric test (Kruskal-Wallis), P<0.01, with Benjamini-Hochberg correction (1473 genes) and independently clustered using Pearson correlation comparing active PTB, latent TB and healthy controls;

FIGS. 3A-3D show the gene array expression results from PAL2, 2 folds up or down expressed, filtered for statistically significant differences in expression between clinical groups using a non-parametric test (Kruskal-Wallis), P<0.01, with Benjamini-Hochberg correction, and then filtered for the presence of the gene ontology term for biological process “immune response” in the gene annotation and independently clustered using Pearson correlation (158 genes). These 158 genes are shown separated into 4 FIGS. 3A-3D) for legibility.

FIG. 3A shows gene array expression results comparing active PTB, latent TB, healthy BCG non-vaccinated controls and healthy BCG vaccinated controls;

FIG. 3B shows gene array expression results comparing active PTB, latent TB, healthy BCG non-vaccinated controls and healthy BCG vaccinated controls;

FIG. 3C shows gene array expression results comparing active PTB, latent TB, healthy BCG non-vaccinated controls and healthy BCG vaccinated controls;

FIG. 3D shows gene array expression results comparing active PTB, latent TB, healthy BCG non-vaccinated controls and healthy BCG vaccinated controls;

FIG. 4 shows the gene array expression results from 42 participants, genes present in at least 2 samples (PAL2), genes 2 folds over or under represented compared with median, Genes selected as TRIMs—clustered by Pearson Correlation comparing active PTB, latent TB, healthy BCG non-vaccinated controls and healthy BCG vaccinated controls;

FIG. 5A shows detail from the gene array expression results from 42 participants, genes present in at least 2 samples (PAL2), genes 2 folds over or under represented compared with median, clustered by Pearson Correlation comparing active PTB, latent TB, healthy BCG non-vaccinated controls and healthy BCG vaccinated controls, showing that inhibitory immunoregulatory ligands (PDL1/CD274, PDL2/CD273) are overexpressed in active TB patients.

FIG. 5B shows the unfiltered gene array expression results that demonstrate that PDL1 is only expressed in active TB patients;

FIG. 6 shows the gene array expression results filtered for genes present in at least 2 samples, 2 folds up or down ‘represented’ compared to median, statistically significantly differentially expressed across groups (P<0.1, Kruskal-Wallis non-parametric test with Bonferroni correction) (46 genes) independently clustered using Pearson correlation, comparing active PTB, latent TB, healthy BCG non-vaccinated controls and healthy BCG vaccinated controls;

FIG. 7 shows the gene array expression results filtered for genes present in at least 2 samples, 2 folds up or down ‘represented’ compared to median, statistically significantly differentially expressed across groups (P<0.05, Kruskal-Wallis non-parametric test with Bonferroni correction) (18 genes) independently clustered using Pearson correlation, comparing active PTB, latent TB, healthy BCG non-vaccinated controls and healthy BCG vaccinated controls;

FIG. 8A shows that the results of merging different statistical filters applied to the list of genes filtered present in at least 2 samples, 2 folds up or down ‘represented’ compared to median, discriminates between all three clinical groups. The transcripts shown are statistically significantly differentially expressed between Latent and healthy (P<0.005, Wilcoxon-Mann-Whitney non-parametric test with no correction) plus the transcripts statistically significantly differentially expressed between Active and healthy (P<0.5, Wilcoxon-Mann-Whitney non-parametric test with Bonferroni correction)—119 genes in total independently clustered using Pearson correlation (clusters of patients/clinical groups are presented horizontally and clusters of genes are presented vertically); These 119 genes are shown separated into 5 further FIGS. 8B-8F) for legibility and to show that subgroups of these genes may also be used to distinguish between different clinical groups (i.e. between Active, Latent and Healthy).

FIG. 8B shows the gene array expression results filtered for genes present in at least 2 samples, 2 folds up or down ‘represented’ compared to median, transcripts statistically significantly differentially expressed between Latent and healthy (P<0.005, Wilcoxon-Mann-Whitney non-parametric test with no correction) PLUS transcripts statistically significantly differentially expressed between Active and healthy (P<0.5, Wilcoxon-Mann-Whitney non-parametric test with Bonferroni correction) (clusters of patients/clinical groups are presented horizontally and clusters of genes are presented vertically);

FIG. 8C shows the gene array expression results filtered for genes present in at least 2 samples, 2 folds up or down ‘represented’ compared to median, transcripts statistically significantly differentially expressed between Latent and healthy (P<0.005, Wilcoxon-Mann-Whitney non-parametric test with no correction) PLUS transcripts statistically significantly differentially expressed between Active and healthy (P<0.5, Wilcoxon-Mann-Whitney non-parametric test with Bonferroni correction);

FIG. 8D shows the gene array expression results filtered for genes present in at least 2 samples, 2 folds up or down ‘represented’ compared to median, transcripts statistically significantly differentially expressed between Latent and healthy (P<0.005, Wilcoxon-Mann-Whitney non-parametric test with no correction) PLUS transcripts statistically significantly differentially expressed between Active and healthy (P<0.5, Wilcoxon-Mann-Whitney non-parametric test with Bonferroni correction) (clusters of patients/clinical groups are presented horizontally and clusters of genes are presented vertically);

FIG. 8E shows the gene array expression results filtered for genes present in at least 2 samples, 2 folds up or down ‘represented’ compared to median, transcripts statistically significantly differentially expressed between Latent and healthy (P<0.005, Wilcoxon-Mann-Whitney non-parametric test with no correction) PLUS transcripts statistically significantly differentially expressed between Active and healthy (P<0.5, Wilcoxon-Mann-Whitney non-parametric test with Bonferroni correction) (clusters of patients/clinical groups are presented horizontally and clusters of genes are presented vertically);

FIG. 8F shows the gene array expression results filtered for genes present in at least 2 samples, 2 folds up or down ‘represented’ compared to median, transcripts statistically significantly differentially expressed between Latent and healthy (P<0.005, Wilcoxon-Mann-Whitney non-parametric test with no correction) PLUS transcripts statistically significantly differentially expressed between Active and healthy (P<0.5, Wilcoxon-Mann-Whitney non-parametric test with Bonferroni correction) (clusters of patients/clinical groups are presented horizontally and clusters of genes are presented vertically);

FIG. 9 shows the gene array expression results from a gene module analysis of PTB(9) vs Control(6): from 5281 genes, filtered for PAL2, statistically significantly differentially expressed between active PTB and healthy controls by Wilcoxon-Mann-Whitney-test, p<0.05, with no multi-test correction; and

FIG. 10 shows the gene array expression results from from a gene module analysis of LTB(9) vs Control(6): from −3137 genes, filtered for PAL2, statistically significantly differentially expressed between active PTB and healthy controls by Wilcoxon-Mann-Whitney-test, p<0.05, with no multi-test correction.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2d ed. 1994); The Cambridge Dictionary of Science and Technology (Walker ed., 1988); The Glossary of Genetics, 5TH ED., R. Rieger et al. (eds.), Springer Verlag (1991); and Hale & Marham, The Harper Collins Dictionary of Biology (1991).

Various biochemical and molecular biology methods are well known in the art. For example, methods of isolation and purification of nucleic acids are described in detail in WO 97/10365; WO 97/27317; Chapter 3 of Laboratory Techniques in Biochemistry and Molecular Biology: Hybridization with Nucleic Acid Probes, Part I. Theory and Nucleic Acid Preparation, (P. Tijssen, ed.) Elsevier, N.Y. (1993); Sambrook, et al., Molecular Cloning: A Laboratory Manual, Cold Spring Harbor Press, N.Y., (1989); and Current Protocols in Molecular Biology, (Ausubel, F. M. et al., eds.) John Wiley & Sons, Inc., New York (1987-1999), including supplements.

Bioinformatics Definitions

As used herein, an “object” refers to any item or information of interest (generally textual, including noun, verb, adjective, adverb, phrase, sentence, symbol, numeric characters, etc.). Therefore, an object is anything that can form a relationship and anything that can be obtained, identified, and/or searched from a source. “Objects” include, but are not limited to, an entity of interest such as gene, protein, disease, phenotype, mechanism, drug, etc. In some aspects, an object may be data, as further described below.

As used herein, a “relationship” refers to the co-occurrence of objects within the same unit (e.g., a phrase, sentence, two or more lines of text, a paragraph, a section of a webpage, a page, a magazine, paper, book, etc.). It may be text, symbols, numbers and combinations, thereof

As used herein, “meta data content” refers to information as to the organization of text in a data source. Meta data can comprise standard metadata such as Dublin Core metadata or can be collection-specific. Examples of metadata formats include, but are not limited to, Machine Readable Catalog (MARC) records used for library catalogs, Resource Description Format (RDF) and the Extensible Markup Language (XML). Meta objects may be generated manually or through automated information extraction algorithms.

As used herein, an “engine” refers to a program that performs a core or essential function for other programs. For example, an engine may be a central program in an operating system or application program that coordinates the overall operation of other programs. The term “engine” may also refer to a program containing an algorithm that can be changed. For example, a knowledge discovery engine may be designed so that its approach to identifying relationships can be changed to reflect new rules of identifying and ranking relationships.

As used herein, “semantic analysis” refers to the identification of relationships between words that represent similar concepts, e.g., though suffix removal or stemming or by employing a thesaurus. “Statistical analysis” refers to a technique based on counting the number of occurrences of each term (word, word root, word stem, n-gram, phrase, etc.). In collections unrestricted as to subject, the same phrase used in different contexts may represent different concepts. Statistical analysis of phrase co-occurrence can help to resolve word sense ambiguity. “Syntactic analysis” can be used to further decrease ambiguity by part-of-speech analysis. As used herein, one or more of such analyses are referred to more generally as “lexical analysis.” “Artificial intelligence (AI)” refers to methods by which a non-human device, such as a computer, performs tasks that humans would deem noteworthy or “intelligent.” Examples include identifying pictures, understanding spoken words or written text, and solving problems.

Terms such “data”, “dataset” and “information” are often used interchangeably, as are “information” and “knowledge.” As used herein, “data” is the most fundamental unit that is an empirical measurement or set of measurements. Data is compiled to contribute to information, but it is fundamentally independent of it and may be combined into a dataset, that is, a set of data. Information, by contrast, is derived from interests, e.g., data (the unit) may be gathered on ethnicity, gender, height, weight and diet for the purpose of finding variables correlated with risk of cardiovascular disease. However, the same data could be used to develop a formula or to create “information” about dietary preferences, i.e., likelihood that certain products in a supermarket have a higher likelihood of selling.

As used herein, the term “database” refers to repositories for raw or compiled data, even if various informational facets can be found within the data fields. A database may include one or more datasets. A database is typically organized so its contents can be accessed, managed, and updated (e.g., the database is dynamic). The term “database” and “source” are also used interchangeably in the present invention, because primary sources of data and information are databases. However, a “source database” or “source data” refers in general to data, e.g., unstructured text and/or structured data that are input into the system for identifying objects and determining relationships. A source database may or may not be a relational database. However, a system database usually includes a relational database or some equivalent type of database which stores values relating to relationships between objects.

As used herein, a “system database” and “relational database” are used interchangeably and refer to one or more collections of data organized as a set of tables containing data fitted into predefined categories. For example, a database table may comprise one or more categories defined by columns (e.g. attributes), while rows of the database may contain a unique object for the categories defined by the columns. Thus, an object such as the identity of a gene might have columns for its presence, absence and/or level of expression of the gene. A row of a relational database may also be referred to as a “set” and is generally defined by the values of its columns. A “domain” in the context of a relational database is a range of valid values a field such as a column may include.

As used herein, a “domain of knowledge” refers to an area of study over which the system is operative, for example, all biomedical data. It should be pointed out that there is advantage to combining data from several domains, for example, biomedical data and engineering data, for this diverse data can sometimes link things that cannot be put together for a normal person that is only familiar with one area or research/study (one domain). A “distributed database” refers to a database that may be dispersed or replicated among different points in a network.

As used herein, “information” refers to a data set that may include numbers, letters, sets of numbers, sets of letters, or conclusions resulting or derived from a set of data. “Data” is then a measurement or statistic and the fundamental unit of information. “Information” may also include other types of data such as words, symbols, text, such as unstructured free text, code, etc. “Knowledge” is loosely defined as a set of information that gives sufficient understanding of a system to model cause and effect. To extend the previous example, information on demographics, gender and prior purchases may be used to develop a regional marketing strategy for food sales while information on nationality could be used by buyers as a guideline for importation of products. It is important to note that there are no strict boundaries between data, information, and knowledge; the three terms are, at times, considered to be equivalent. In general, data comes from examining, information comes from correlating, and knowledge comes from modeling.

As used herein, “a program” or “computer program” refers generally to a syntactic unit that conforms to the rules of a particular programming language and that is composed of declarations and statements or instructions, divisible into, “code segments” needed to solve or execute a certain function, task, or problem. A programming language is generally an artificial language for expressing programs.

As used herein, a “system” or a “computer system” generally refers to one or more computers, peripheral equipment, and software that perform data processing. A “user” or “system operator” in general includes a person, that uses a computer network accessed through a “user device” (e.g., a computer, a wireless device, etc) for the purpose of data processing and information exchange. A “computer” is generally a functional unit that can perform substantial computations, including numerous arithmetic operations and logic operations without human intervention.

As used herein, “application software” or an “application program” refers generally to software or a program that is specific to the solution of an application problem. An “application problem” is generally a problem submitted by an end user and requiring information processing for its solution.

As used herein, a “natural language” refers to a language whose rules are based on current usage without being specifically prescribed, e.g., English, Spanish or Chinese. As used herein, an “artificial language” refers to a language whose rules are explicitly established prior to its use, e.g., computer-programming languages such as C, C++, Java, BASIC, FORTRAN, or COBOL.

As used herein, “statistical relevance” refers to using one or more of the ranking schemes (0/E ratio, strength, etc.), where a relationship is determined to be statistically relevant if it occurs significantly more frequently than would be expected by random chance.

As used herein, the terms “coordinately regulated genes” or “transcriptional modules” are used interchangeably to refer to grouped, gene expression profiles (e.g., signal values associated with a specific gene sequence) of specific genes. Each transcriptional module correlates two key pieces of data, a literature search portion and actual empirical gene expression value data obtained from a gene microarray. The set of genes that is selected into a transcriptional modules is based on the analysis of gene expression data (module extraction algorithm described above). Additional steps are taught by Chaussabel, D. & Sher, A Mining microarray expression data by literature profiling. Genome Biol 3, RESEARCH0055 (2002), (http://genomebiology.com/2002/3/10/research/0055) relevant portions incorporated herein by reference and expression data obtained from a disease or condition of interest, e.g., Systemic Lupus erythematosus, arthritis, lymphoma, carcinoma, melanoma, acute infection, autoimmune disorders, autoinflammatory disorders, etc.).

The Table below lists examples of keywords that were used to develop the literature search portion or contribution to the transcription modules. The skilled artisan will recognize that other terms may easily be selected for other conditions, e.g., specific cancers, specific infectious disease, transplantation, etc. For example, genes and signals for those genes associated with T cell activation are described hereinbelow as Module ID “M 2.8” in which certain keywords (e.g., Lymphoma, T-cell, CD4, CD8, TCR, Thymus, Lymphoid, IL2) were used to identify key T-cell associated genes, e.g., T-cell surface markers (CD5, CD6, CD7, CD26, CD28, CD96); molecules expressed by lymphoid lineage cells (lymphotoxin beta, IL2-inducible T-cell kinase, TCF7; and T-cell differentiation protein mal, GATA3, STAT5B). Next, the complete module is developed by correlating data from a patient population for these genes (regardless of platform, presence/absence and/or up or downregulation) to generate the transcriptional module. In some cases, the gene profile does not match (at this time) any particular clustering of genes for these disease conditions and data, however, certain physiological pathways (e.g., cAMP signaling, zinc-finger proteins, cell surface markers, etc.) are found within the “Underdetermined” modules. In fact, the gene expression data set may be used to extract genes that have coordinated expression prior to matching to the keyword search, i.e., either data set may be correlated prior to cross-referencing with the second data set.

TABLE 1
Transcriptional Modules

| Example | | |
|---|---|---|
| Module I.D. | Example Keyword selection | Gene Profile Assessment |
| M 1.1 | Ig, Immunoglobulin, Bone, | Plasma cells: Includes genes encoding for Immunoglobulin chains |
| | Marrow, PreB, IgM, Mu. | (e.g. IGHM, IGJ, IGLL1, IGKC, IGHD) and the plasma cell marker |
| | | CD38. |
| M 1.2 | Platelet, Adhesion, | Platelets: Includes genes encoding for platelet glycoproteins |
| | Aggregation, Endothelial, | (ITGA2B, ITGB3, GP6, GP1A/B), and platelet-derived immune |
| | Vascular | mediators such as PPPB (pro-platelet basic protein) and PF4 (platelet |
| | | factor 4). |
| M 1.3 | Immunoreceptor, BCR, B- | B-cells: Includes genes encoding for B-cell surface markers (CD72, |
| | cell, IgG | CD79A/B, CD19, CD22) and other B-cell associated molecules: |
| | | Early B-cell factor (EBF), B-cell linker (BLNK) and B lymphoid |
| | | tyrosine kinase (BLK). |
| M 1.4 | Replication, Repression, | Undetermined. This set includes regulators and targets of cAMP |
| | Repair, CREB, Lymphoid, | signaling pathway (JUND, ATF4, CREM, PDE4, NR4A2, VIL2), as |
| | TNF-alpha | well as repressors of TNF-alpha mediated NF-KB activation (CYLD, |
| | | ASK, TNFAIP3). |
| M 1.5 | Monocytes, Dendritic, MHC, | Myeloid lineage: Includes molecules expressed by cells of the |
| | Costimulatory, TLR4, | myeloid lineage (CD86, CD163, FCGR2A), some of which being |
| | MYD88 | involved in pathogen recognition (CD14, TLR2, MYD88). This set |
| | | also includes TNF family members (TNFR2, BAFF). |
| M 1.6 | Zinc, Finger, P53, RAS | Undetermined. This set includes genes encoding for signaling |
| | | molecules, e.g., the zinc finger containing inhibitor of activated |
| | | STAT (PIAS1 and PIAS2), or the nuclear factor of activated T-cells |
| | | NFATC3. |
| M 1.7 | Ribosome, Translational, | MHC/Ribosomal proteins: Almost exclusively formed by genes |
| | 40S, 60S, HLA | encoding MHC class I molecules (HLA-A,B,C,G,E) + Beta 2- |
| | | microglobulin (B2M) or Ribosomal proteins (RPLs, RPSs). |
| M 1.8 | Metabolism, Biosynthesis, | Undetermined. Includes genes encoding metabolic enzymes (GLS, |
| | Replication, Helicase | NSF1, NAT1) and factors involved in DNA replication (PURA, |
| | | TERF2, EIF2S1). |
| M 2.1 | NK, Killer, Cytolytic, CD8, | Cytotoxic cells: Includes cytotoxic T-cells and NK-cells surface |
| | Cell-mediated, T-cell, CTL, | markers (CD8A, CD2, CD160, NKG7, KLRs), cytolytic molecules |
| | IFN-g | (granzyme, perforin, granulysin), chemokines (CCL5, XCL1) and |
| | | CTL/NK-cell associated molecules (CTSW). |
| M 2.2 | Granulocytes, Neutrophils, | Neutrophils: This set includes innate molecules that are found in |
| | Defense, Myeloid, Marrow | neutrophil granules (Lactotransferrin: LTF, defensin: DEAF1, |
| | | Bacterial Permeability Increasing protein: BPI, Cathelicidin |
| | | antimicrobial protein: CAMP). |
| M 2.3 | Erythrocytes, Red, Anemia, | Erythrocytes: Includes hemoglobin genes (HGBs) and other |
| | Globin, Hemoglobin | erythrocyte-associated genes (erythrocytic alkirin: ANK1, |
| | | Glycophorin C: GYPC, hydroxymethylbilane synthase: HMBS, |
| | | erythroid associated factor: ERAF). |
| M 2.4 | Ribonucleoprotein, 60S, | Ribosomal proteins: Including genes encoding ribosomal proteins |
| | nucleolus, Assembly, | (RPLs, RPSs), Eukaryotic Translation Elongation factor family |
| | Elongation | members (EEFs) and Nucleolar proteins (NPM1, NOAL2, NAP1L1). |
| M 2.5 | Adenoma Interstitial, | Undetermined. This module includes genes encoding immune-related |
| | Mesenchyme, Dendrite, | (CD40, CD80, CXCL12, IFNA5, IL4R) as well as cytoskeleton- |
| | Motor | related molecules (Myosin, Dedicator of Cytokenesis, Syndecan 2, |
| | | Plexin C1, Distrobrevin). |
| M 2.6 | Granulocytes, Monocytes, | Myeloid lineage: Related to M 1.5. Includes genes expressed in |
| | Myeloid, ERK, Necrosis | myeloid lineage cells (IGTB2/CD18, Lymphotoxin beta receptor, |
| | | Myeloid related proteins 8/14 Formyl peptide receptor 1), such as |
| | | Monocytes and Neutrophils: |
| M 2.7 | No keywords extracted. | Undetermined. This module is largely composed of transcripts with |
| | | no known function. Only 20 genes associated with literature, |
| | | including a member of the chemokine-like factor superfamily |
| | | (CKLFSF8). |
| M 2.8 | Lymphoma, T-cell, CD4, | T-cells: Includes T-cell surface markers (CD5, CD6, CD7, CD26, |
| | CD8, TCR, Thymus, | CD28, CD96) and molecules expressed by lymphoid lineage cells |
| | Lymphoid, IL2 | (lymphotoxin beta, IL2-inducible T-cell kinase, TCF7, T-cell |
| | | differentiation protein mal, GATA3, STAT5B). |
| M 2.9 | ERK, Transactivation, | Undetermined. Includes genes encoding molecules that associate to |
| | Cytoskeletal, MAPK, JNK | the cytoskeleton (Actin related protein 2/3, MAPK1, MAP3K1, |
| | | RAB5A). Also present are T-cell expressed genes (FAS, |
| | | ITGA4/CD49D, ZNF1A1). |
| M 2.10 | Myeloid, Macrophage, | Undetermined. Includes genes encoding for Immune-related cell |
| | Dendritic, Inflammatory, | surface molecules (CD36, CD86, LILRB), cytokines (IL15) and |
| | Interleukin | molecules involved in signaling pathways (FYB, TICAM2-Toll-like |
| | | receptor pathway). |
| M 2.11 | Replication, Repress, RAS, | Undetermined. Includes kinases (UHMK1, CSNK1G1, CDK6, |
| | Autophosphorylation, | WNK1, TAOK1, CALM2, PRKCI, ITPKB, SRPK2, STK17B, |
| | Oncogenic | DYRK2, PIK3R1, STK4, CLK4, PKN2) and RAS family members |
| | | (G3BP, RAB14, RASA2, RAP2A, KRAS). |
| M 3.1 | ISRE, Influenza, Antiviral, | Interferon-inducible: This set includes interferon-inducible genes: |
| | IFN-gamma, IFN-alpha, | antiviral molecules (OAS1/2/3/L, GBP1, G1P2, EIF2AK2/PKR, |
| | Interferon | MX1, PML), chemokines (CXCL10/IP-10), signaling molecules |
| | | (STAT1, STAt2, IRF7, ISGF3G). |
| M 3.2 | TGF-beta, TNF, | Inflammation I: Includes genes encoding molecules involved in |
| | Inflammatory, Apoptotic, | inflammatory processes (e.g., IL8, ICAM1, C5R1, CD44, PLAUR, |
| | Lipopolysaccharide | IL1A, CXCL16), and regulators of apoptosis (MCL1, FOXO3A, |
| | | RARA, BCL3/6/2A1, GADD45B). |
| M 3.3 | Granulocyte, Inflammatory, | Inflammation II: Includes molecules inducing or inducible by |
| | Defense, Oxidize, Lysosomal | Granulocyte-Macrophage CSF (SPI1, IL18, ALOX5, ANPEP), as |
| | | well as lysosomal enzymes (PPT1, CTSB/S, CES1, NEU1, ASAH1, |
| | | LAMP2, CAST). |
| M 3.4 | No keyword extracted | Undetermined. Includes protein phosphates (PPP1R12A, PTPRC, |
| | | PPP1CB, PPM1B) and phosphoinositide 3-kinase (PI3K) family |
| | | members (PIK3CA, PIK32A, PIP5K3). |
| M 3.5 | No keyword extracted | Undetermined. Composed of only a small number of transcripts. |
| | | Includes hemoglobin genes (HBA1, HBA2, HBB). |
| M 3.6 | Complement, Host, | Undetermined. Large set that includes T-cell surface markers |
| | Oxidative, Cytoskeletal, T- | (CD101, CD102, CD103) as well as molecules ubiquitously |
| | cell | expressed among blood leukocytes (CXRCR1: fraktalkine receptor, |
| | | CD47, P-selectin ligand). |
| M 3.7 | Spliceosome, Methylation, | Undetermined. Includes genes encoding proteasome subunits |
| | Ubiquitin, Beta-catenin | (PSMA2/5, PSMB5/8); ubiquitin protein ligases HIP2, STUB1, as |
| | | well as components of ubiqutin ligase complexes (SUGT1). |
| M 3.8 | CDC, TCR, CREB, | Undetermined. Includes genes encoding for several enzymes: |
| | Glycosylase | aminomethyltransferase, arginyltransferase, asparagines synthetase, |
| | | diacylglycerol kinase, inositol phosphatases, methyltransferases, |
| | | helicases . . . |
| M 3.9 | Chromatin, Checkpoint, | Undetermined. Includes genes encoding for protein kinases |
| | Replication, Transactivation | (PRKPIR, PRKDC, PRKCI) and phosphatases (e.g., PTPLB, |
| | | PPP1R8/2CB). Also includes RAS oncogene family members and |
| | | the NK cell receptor 2B4 (CD244). |

Biological Definitions

As used herein, the term “array” refers to a solid support or substrate with one or more peptides or nucleic acid probes attached to the support. Arrays typically have one or more different nucleic acid or peptide probes that are coupled to a surface of a substrate in different, known locations. These arrays, also described as “microarrays” or “gene-chips” that may have 10,000; 20,000, 30,000; or 40,000 different identifiable genes based on the known genome, e.g., the human genome. These pan-arrays are used to detect the entire “transcriptome” or transcriptional pool of genes that are expressed or found in a sample, e.g., nucleic acids that are expressed as RNA, mRNA and the like that may be subjected to RT and/or RT-PCR to made a complementary set of DNA replicons. Arrays may be produced using mechanical synthesis methods, light directed synthesis methods and the like that incorporate a combination of non-lithographic and/or photolithographic methods and solid phase synthesis methods.

Various techniques for the synthesis of these nucleic acid arrays have been described, e.g., fabricated on a surface of virtually any shape or even a multiplicity of surfaces. Arrays may be peptides or nucleic acids on beads, gels, polymeric surfaces, fibers such as fiber optics, glass or any other appropriate substrate. Arrays may be packaged in such a manner as to allow for diagnostics or other manipulation of an all inclusive device, see for example, U.S. Pat. No. 6,955,788, relevant portions incorporated herein by reference.

As used herein, the term “disease” refers to a physiological state of an organism with any abnormal biological state of a cell. Disease includes, but is not limited to, an interruption, cessation or disorder of cells, tissues, body functions, systems or organs that may be inherent, inherited, caused by an infection, caused by abnormal cell function, abnormal cell division and the like. A disease that leads to a “disease state” is generally detrimental to the biological system, that is, the host of the disease. With respect to the present invention, any biological state, such as an infection (e.g., viral, bacterial, fungal, helminthic, etc.), inflammation, autoinflammation, autoimmunity, anaphylaxis, allergies, premalignancy, malignancy, surgical, transplantation, physiological, and the like that is associated with a disease or disorder is considered to be a disease state. A pathological state is generally the equivalent of a disease state.

Disease states may also be categorized into different levels of disease state. As used herein, the level of a disease or disease state is an arbitrary measure reflecting the progression of a disease or disease state as well as the physiological response upon, during and after treatment. Generally, a disease or disease state will progress through levels or stages, wherein the affects of the disease become increasingly severe. The level of a disease state may be impacted by the physiological state of cells in the sample.

As used herein, the terms “therapy” or “therapeutic regimen” refer to those medical steps taken to alleviate or alter a disease state, e.g., a course of treatment intended to reduce or eliminate the affects or symptoms of a disease using pharmacological, surgical, dietary and/or other techniques. A therapeutic regimen may include a prescribed dosage of one or more drugs or surgery. Therapies will most often be beneficial and reduce the disease state but in many instances the effect of a therapy will have non-desirable or side-effects. The effect of therapy will also be impacted by the physiological state of the host, e.g., age, gender, genetics, weight, other disease conditions, etc.

As used herein, the term “pharmacological state” or “pharmacological status” refers to those samples that will be, are and/or were treated with one or more drugs, surgery and the like that may affect the pharmacological state of one or more nucleic acids in a sample, e.g., newly transcribed, stabilized and/or destabilized as a result of the pharmacological intervention. The pharmacological state of a sample relates to changes in the biological status before, during and/or after drug treatment and may serve a diagnostic or prognostic function, as taught herein. Some changes following drug treatment or surgery may be relevant to the disease state and/or may be unrelated side-effects of the therapy. Changes in the pharmacological state are the likely results of the duration of therapy, types and doses of drugs prescribed, degree of compliance with a given course of therapy, and/or un-prescribed drugs ingested.

As used herein, the term “biological state” refers to the state of the transcriptome (that is the entire collection of RNA transcripts) of the cellular sample isolated and purified for the analysis of changes in expression. The biological state reflects the physiological state of the cells in the sample by measuring the abundance and/or activity of cellular constituents, characterizing according to morphological phenotype or a combination of the methods for the detection of transcripts.

As used herein, the term “expression profile” refers to the relative abundance of RNA, DNA or protein abundances or activity levels. The expression profile can be a measurement for example of the transcriptional state or the translational state by any number of methods and using any of a number of gene-chips, gene arrays, beads, multiplex PCR, quantitiative PCR, run-on assays, Northern blot analysis, Western blot analysis, protein expression, fluorescence activated cell sorting (FACS), enzyme linked immunosorbent assays (ELISA), chemiluminescence studies, enzymatic assays, proliferation studies or any other method, apparatus and system for the determination and/or analysis of gene expression that are readily commercially available.

As used herein, the term “transcriptional state” of a sample includes the identities and relative abundances of the RNA species, especially mRNAs present in the sample. The entire transcriptional state of a sample, that is the combination of identity and abundance of RNA, is also referred to herein as the transcriptome. Generally, a substantial fraction of all the relative constituents of the entire set of RNA species in the sample are measured.

As used herein, the term “modular transcriptional vectors” refers to transcriptional expression data that reflects the “proportion of differentially expressed genes.” For example, for each module the proportion of transcripts differentially expressed between at least two groups (e.g. healthy subjects vs patients). This vector is derived from the comparison of two groups of samples. The first analytical step is used for the selection of disease-specific sets of transcripts within each module. Next, there is the “expression level.” The group comparison for a given disease provides the list of differentially expressed transcripts for each module. It was found that different diseases yield different subsets of modular transcripts. With this expression level it is then possible to calculate vectors for each module(s) for a single sample by averaging expression values of disease-specific subsets of genes identified as being differentially expressed. This approach permits the generation of maps of modular expression vectors for a single sample, e.g., those described in the module maps disclosed herein. These vector module maps represent an averaged expression level for each module (instead of a proportion of differentially expressed genes) that can be derived for each sample.

Using the present invention it is possible to identify and distinguish diseases not only at the module-level, but also at the gene-level; i.e., two diseases can have the same vector (identical proportion of differentially expressed transcripts, identical “polarity”), but the gene composition of the vector can still be disease-specific. Gene-level expression provides the distinct advantage of greatly increasing the resolution of the analysis. Furthermore, the present invention takes advantage of composite transcriptional markers. As used herein, the term “composite transcriptional markers” refers to the average expression values of multiple genes (subsets of modules) as compared to using individual genes as markers (and the composition of these markers can be disease-specific). The composite transcriptional markers approach is unique because the user can develop multivariate microarray scores to assess disease severity in patients with, e.g., SLE, or to derive expression vectors disclosed herein. Most importantly, it has been found that using the composite modular transcriptional markers of the present invention the results found herein are reproducible across microarray platform, thereby providing greater reliability for regulatory approval.

Gene expression monitoring systems for use with the present invention may include customized gene arrays with a limited and/or basic number of genes that are specific and/or customized for the one or more target diseases. Unlike the general, pan-genome arrays that are in customary use, the present invention provides for not only the use of these general pan-arrays for retrospective gene and genome analysis without the need to use a specific platform, but more importantly, it provides for the development of customized arrays that provide an optimal gene set for analysis without the need for the thousands of other, non-relevant genes. One distinct advantage of the optimized arrays and modules of the present invention over the existing art is a reduction in the financial costs (e.g., cost per assay, materials, equipment, time, personnel, training, etc.), and more importantly, the environmental cost of manufacturing pan-arrays where the vast majority of the data is irrelevant. The modules of the present invention allow for the first time the design of simple, custom arrays that provide optimal data with the least number of probes while maximizing the signal to noise ratio. By eliminating the total number of genes for analysis, it is possible to, e.g., eliminate the need to manufacture thousands of expensive platinum masks for photolithography during the manufacture of pan-genetic chips that provide vast amounts of irrelevant data. Using the present invention it is possible to completely avoid the need for microarrays if the limited probe set(s) of the present invention are used with, e.g., digital optical chemistry arrays, ball bead arrays, beads (e.g., Luminex), multiplex PCR, quantitiative PCR, run-on assays, Northern blot analysis, or even, for protein analysis, e.g., Western blot analysis, 2-D and 3-D gel protein expression, MALDI, MALDI-TOF, fluorescence activated cell sorting (FACS) (cell surface or intracellular), enzyme linked immunosorbent assays (ELISA), chemiluminescence studies, enzymatic assays, proliferation studies or any other method, apparatus and system for the determination and/or analysis of gene expression that are readily commercially available.

The “molecular fingerprinting system” of the present invention may be used to facilitate and conduct a comparative analysis of expression in different cells or tissues, different subpopulations of the same cells or tissues, different physiological states of the same cells or tissue, different developmental stages of the same cells or tissue, or different cell populations of the same tissue against other diseases and/or normal cell controls. In some cases, the normal or wild-type expression data may be from samples analyzed at or about the same time or it may be expression data obtained or culled from existing gene array expression databases, e.g., public databases such as the NCBI Gene Expression Omnibus database.

As used herein, the term “differentially expressed” refers to the measurement of a cellular constituent (e.g., nucleic acid, protein, enzymatic activity and the like) that varies in two or more samples, e.g., between a disease sample and a normal sample. The cellular constituent may be on or off (present or absent), upregulated relative to a reference or downregulated relative to the reference. For use with gene-chips or gene-arrays, differential gene expression of nucleic acids, e.g., mRNA or other RNAs (miRNA, siRNA, hnRNA, rRNA, tRNA, etc.) may be used to distinguish between cell types or nucleic acids. Most commonly, the measurement of the transcriptional state of a cell is accomplished by quantitative reverse transcriptase (RT) and/or quantitative reverse transcriptase-polymerase chain reaction (RT-PCR), genomic expression analysis, post-translational analysis, modifications to genomic DNA, translocations, in situ hybridization and the like.

For some disease states it is possible to identify cellular or morphological differences, especially at early levels of the disease state. The present invention avoids the need to identify those specific mutations or one or more genes by looking at modules of genes of the cells themselves or, more importantly, of the cellular RNA expression of genes from immune effector cells that are acting within their regular physiologic context, that is, during immune activation, immune tolerance or even immune anergy. While a genetic mutation may result in a dramatic change in the expression levels of a group of genes, biological systems often compensate for changes by altering the expression of other genes. As a result of these internal compensation responses, many perturbations may have minimal effects on observable phenotypes of the system but profound effects to the composition of cellular constituents. Likewise, the actual copies of a gene transcript may not increase or decrease, however, the longevity or half-life of the transcript may be affected leading to greatly increases protein production. The present invention eliminates the need of detecting the actual message by, in one embodiment, looking at effector cells (e.g., leukocytes, lymphocytes and/or sub-populations thereof) rather than single messages and/or mutations.

The skilled artisan will appreciate readily that samples may be obtained from a variety of sources including, e.g., single cells, a collection of cells, tissue, cell culture and the like. In certain cases, it may even be possible to isolate sufficient RNA from cells found in, e.g., urine, blood, saliva, tissue or biopsy samples and the like. In certain circumstances, enough cells and/or RNA may be obtained from: mucosal secretion, feces, tears, blood plasma, peritoneal fluid, interstitial fluid, intradural, cerebrospinal fluid, sweat or other bodily fluids. The nucleic acid source, e.g., from tissue or cell sources, may include a tissue biopsy sample, one or more sorted cell populations, cell culture, cell clones, transformed cells, biopies or a single cell. The tissue source may include, e.g., brain, liver, heart, kidney, lung, spleen, retina, bone, neural, lymph node, endocrine gland, reproductive organ, blood, nerve, vascular tissue, and olfactory epithelium.

The present invention includes the following basic components, which may be used alone or in combination, namely, one or more data mining algorithms; one or more module-level analytical processes; the characterization of blood leukocyte transcriptional modules; the use of aggregated modular data in multivariate analyses for the molecular diagnostic/prognostic of human diseases; and/or visualization of module-level data and results. Using the present invention it is also possible to develop and analyze composite transcriptional markers, which may be further aggregated into a single multivariate score.

An explosion in data acquisition rates has spurred the development of mining tools and algorithms for the exploitation of microarray data and biomedical knowledge. Approaches aimed at uncovering the modular organization and function of transcriptional systems constitute promising methods for the identification of robust molecular signatures of disease. Indeed, such analyses can transform the perception of large scale transcriptional studies by taking the conceptualization of microarray data past the level of individual genes or lists of genes.

The present inventors have recognized that current microarray-based research is facing significant challenges with the analysis of data that are notoriously “noisy,” that is, data that is difficult to interpret and does not compare well across laboratories and platforms. A widely accepted approach for the analysis of microarray data begins with the identification of subsets of genes differentially expressed between study groups. Next, the users try subsequently to “make sense” out of resulting gene lists using pattern discovery algorithms and existing scientific knowledge.

Rather than deal with the great variability across platforms, the present inventors have developed a strategy that emphasized the selection of biologically relevant genes at an early stage of the analysis. Briefly, the method includes the identification of the transcriptional components characterizing a given biological system for which an improved data mining algorithm was developed to analyze and extract groups of coordinately expressed genes, or transcriptional modules, from large collections of data.

Pulmonary tuberculosis (PTB) is a major and increasing cause of morbidity and mortality worldwide caused by Mycobacterium tuberculosis (M. tuberculosis). However, the majority of individuals infected with M. tuberculosis remain asymptomatic, retaining the infection in a latent form and it is thought that this latent state is maintained by an active immune response. Blood is the pipeline of the immune system, and as such is the ideal biologic material from which the health and immune status of an individual can be established. Here, using microarray technology to assess the activity of the entire genome in blood cells, we identified distinct and reciprocal blood transcriptional biomarker signatures in patients with active pulmonary tuberculosis and latent tuberculosis. These signatures were also distinct from those in control individuals. The signature of latent tuberculosis, which showed an over-representation of immune cytotoxic gene expression in whole blood, may help to determine protective immune factors against M. tuberculosis infection, since these patients are infected but most do not develop overt disease. This distinct transcriptional biomarker signature from active and latent TB patients may be also used to diagnose infection, and to monitor response to treatment with anti-mycobacterial drugs. In addition the signature in active tuberculosis patients will help to determine factors involved in immunopathogenesis and possibly lead to strategies for immune therapeutic intervention. This invention relates to a previous application that claimed the use of blood transcriptional biomarkers for the diagnosis of infections. However, this previous application did not disclose the existence of biomarkers for active and latent tuberculosis and focused rather on children with other acute infections (Ramillo, Blood, 2007).

The present identification of a transcriptional signature in blood from latent versus active TB patients can be used to test for patients with suspected Mycobacterium tuberculosis infection as well as for health screening/early detection of the disease. The invention also permits the evaluation of the response to treatment with anti-mycobacterial drugs. In this context, a test would also be particularly valuable in the context of drug trials, and particularly to assess drug treatments in Multi-Drug Resistant patients. Furthermore, the present invention may be used to obtain immediate, intermediate and long term data from the immune signature of latent tuberculosis to better define a protective immune response during vaccination trials. Also, the signature in active tuberculosis patients will help to determine factors involved in immunopathogenesis and possibly lead to strategies for immune therapeutic intervention.

Blood represents a reservoir and a migration compartment for cells of the innate and the adaptive immune systems, including either neutrophils, dendritic cells and monocytes, or B and T lymphocytes, respectively, which during infection will have been exposed to infectious agents in the tissue. For this reason whole blood from infected individuals provides an accessible source of clinically relevant material where an unbiased molecular phenotype can be obtained using gene expression microarrays as previously described for the study of cancer in tissues (Alizadeh A A., 2000; Golub, T R., 1999; Bittner, 2000), and autoimmunity (Bennet, 2003; Baechler, E C, 2003; Burczynski, M E, 2005; Chaussabel, D., 2005; Cobb, J P., 2005; Kaizer, E C., 2007; Allantaz, 2005; Allantaz, 2007), and inflammation (Thach, D C., 2005) and infectious disease (Ramillo, Blood, 2007) in blood or tissue (Bleharski, J R et al., 2003). Microarray analyses of gene expression in blood leucocytes have identified diagnostic and prognostic gene expression signatures, which have led to a better understanding of mechanisms of disease onset and responses to treatment (Bennet, L 2003; Rubins, K H., 2004; Baechler, E C, 2003; Pascual, V., 2005; Allantaz, F., 2007; Allantaz, F., 2007). These microarray approaches have been attempted for the study of active and latent TB but as yet have yielded small numbers of differentially expressed genes only (Jacobsen, M., Kaufmann, S H., 2006; Mistry, R, Lukey, P T, 2007), and in relatively small numbers of patients (Mistry, R., 2007), which may not be robust enough to distinguish between other inflammatory and infectious diseases.

To define an immune signature in TB, the blood of active and latent TB patients and controls were analyzed; patients were selected using very stringent clinical criteria. Patients were recruited from London, UK, where numbers of active TB cases are increasing, and most importantly where the risk of confounding coinfections is minimal, to yield a robust signature that may distinguish latent from active TB. Microarrays were used to analyze the whole genome and subsequent data mining revealed a large number of genes found to be differentially expressed at a statistically significant level across all groups of patients, including active and latent TB patients and healthy controls. Next, a novel approach based on a modular data mining strategy was used, this approach provided a basis for the selection of clinically-relevant transcriptional biomarkers for the analysis of blood microarray transcriptional profiles in SLE and other diseases, and improved our understanding of disease pathogenesis (Chaussabel, 2008, Immunity). The module maps defined in this study provide a means to organize and reduce the dimension of complex data, whilst still retaining the large number of genes expressed in human blood, thus allowing visualization of specific disease fingerprints (Chaussabel, 2008, Immunity). Using this modular approach clearly defined modular transcriptional signatures were obtained that are distinct and reciprocal in the whole blood of active and latent TB patients, and which also differ from healthy controls. The biomarkers described herein are improve the diagnosis of PTB, and furthermore will help to define host factors important in the protection against M. tuberculosis in latent TB patients, and those involved in the immunopathogenesis of active TB, and thus be used to reduce and manage TB disease.

Patients, Materials and Methods

Participant recruitment and Patient characterization: Participants were recruited from St. Mary's Hospital TB Clinic, Imperial College Healthcare NHS Trust, London, with healthy controls recruited from volunteers at the National Institute for Medical Research (NIMR), Mill Hill, London. The study was approved by the local NHS Research Ethics Committee at St Marys Hospital (LREC), London, UK. All participants (aged 18 and over) gave written informed consent. Strict clinical criteria were satisfied before recruited participants had their provisional study grouping confirmed and were only then allocated to the final group for analysis. The patient and control cohorts were as follows: (i) Active PTB based on clinical diagnosis subsequently confirmed by laboratory isolation of M. tuberculosis on mycobacterial culture; (ii) Latent TB—defined by a positive tuberculin skin test (TST, Using 2TU tuberculin (Serum Statens Institute, Copenhagen, Denmark) ≧6mm if BCG unvaccinated, ≧15mm if BCG vaccinated, together with a positive result using an Interferon Gamma Release Assay (IGRA, specifically the Quantiferon-TB Gold In-tube assay, Cellestis, Australia). This IGRA assay measured reactivity to antigens (ESAT-6/CFP-10/TB 7.7-present in M. tuberculosis but not in most environmental mycobacteria or the M. bovis BCG vaccine) by IFN-γ release from whole blood. Latent TB patients also had to have evidence of exposure to infectious TB cases, either through close household or workplace contact, or as recent ‘new entrants’ from endemic areas; Patients with incidental findings of TST positivity without evidence of exposure to infected persons, were not eligible for inclusion in the study (iii) Healthy volunteer controls (BCG vaccinated and unvaccinated, ≦14 mm or ≦5 mm by TST respectively; and negative by IGRA). Participants who were pregnant, known to be immunosuppressed, taking immunosuppressive therapies or have diabetes, or autoimmune disease were also ineligible and excluded from this initial study. HIV positive individuals (Only 1% of the TB patients in London present with previously undiagnosed HIV) were excluded from the study. Blood from active and latent PTB patients was collected for the study before any anti-mycobacterial drugs were administered, and then subsequently at set time intervals for the longitudinal part of the study for later study.

Detailed clinical information was collected prospectively for every participant and has been entered into a web-accessible database developed by the present inventors. Using this recorded clinical data, and immune-based assays as described above, 15 out of 58 participants were excluded from the study as they did not meet the standard criteria for the study. This resulted in cohorts of 6 BCG unvaccinated healthy volunteers; 6 BCG vaccinated healthy volunteers, 17 latent TB patients and 14 active PTB patients, all of these samples were then used for RNA isolation. One sample from an active TB patient did not yield sufficient globin reduced RNA after processing to proceed and was therefore excluded from the final analysis.

RNA sampling, extraction, processing for microarray: Whole blood from the above patient cohorts was collected into Tempus tubes (Applied Biosystems, Foster City, Calif., USA) and stored between −20° C. and −80° C. before RNA extraction. Total RNA was isolated using the PerfectPure RNA Blood kit (5 PRIME Inc, Gaithersburg, Md., USA). Samples were homogenized with 100% cold ethanol, vortexed, then centrifuged at 4000 g for 60 minutes at 0° C., and the supernatant discarded. 300 μl lysis solution was then added to the pellet and vortexed. RNA binding, Dnase treatment, wash and RNA elution steps were then performed according to the manufacturer's instructions. Isolated total RNA was then globin reduced using the GLOBINclear™ 96-well format kit (Ambion, Austin, Tex., USA) according to the manufacturer's instructions. Total and globin-reduced RNA integrity was assessed using an Agilent 2100 Bioanalyzer (Agilent, Palo Alto, Calif.). One sample from an active TB patient did not yield sufficient globin reduced RNA after processing to proceed and was therefore excluded from the final analysis. Biotinylated, amplified RNA targets (cRNA) were then prepared from the globin-reduced RNA using the Illumina CustomPrep RNA amplification kit (Ambion, Austin, Tex., USA). Labeled cRNA was hybridized overnight to Sentrix Human-6 V2 BeadChip array (>48,000 probes, Illumina Inc, San Diego, Calif., USA), washed, blocked, stained and scanned on an Illumina BeadStation 500 following the manufacturer's protocols. Illumina's BeadStudio version 2 software was used to generate signal intensity values from the scans, substract background, and scale each microarray to the median average intensity for all samples (per-chip normalization). This normalized data was used for all subsequent data analysis.

Microarray data analysis: A gene expression analysis software program, Genespring, version 7.1.3 (Agilent), was used to perform statistical analysis and hierarchical clustering of samples. Differentially expressed genes were selected and clustered as described in Results and Figure legends.

Results and Discussion.

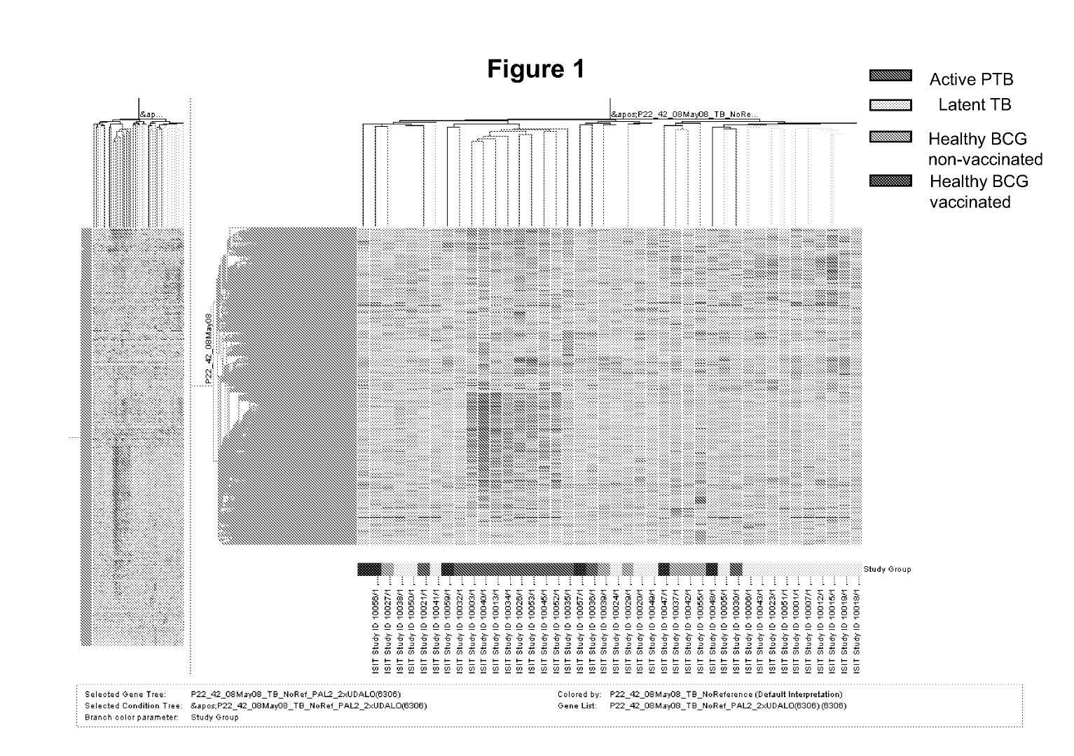

Blood signatures distinguish active and latent TB patients from each other, and from healthy control individuals: To determine whether blood sampled from patients with active and latent TB carry gene expression signatures that allow discrimination between active and latent TB as compared to healthy controls, a step-wise analysis was conducted. After filtering out undetected transcripts and genes with a deviation from the median of less than 2 fold, i.e. with a flat profile, 6269 genes were used for unsupervised clustering analyses by Pearson correlation of the expression profiles obtained from the whole blood RNA samples from active and latent TB and healthy controls (FIG. 1). This unsupervised analysis identified distinct signatures, which were found to correspond to distinct clinical phenotypes: in patients with active pulmonary TB (active PTB); and: in individuals with latent tuberculosis (latent TB). The grouping of samples was not perfect (10 of 13 patients with active TB, and 11 of 17 patients with latent TB). Nonetheless, the majority of active PTB and latent TB patients in this group from the training set of patients appeared to have clear and distinct transcriptional signatures. Importantly these signatures appeared to be represented across the broad number of ethnicities collected for the study, including White, Black African, Asian Indian, Asian Bangladeshi, Asian Other, White Irish, Mixed White, Black Caribbean (details of this data are not shown).

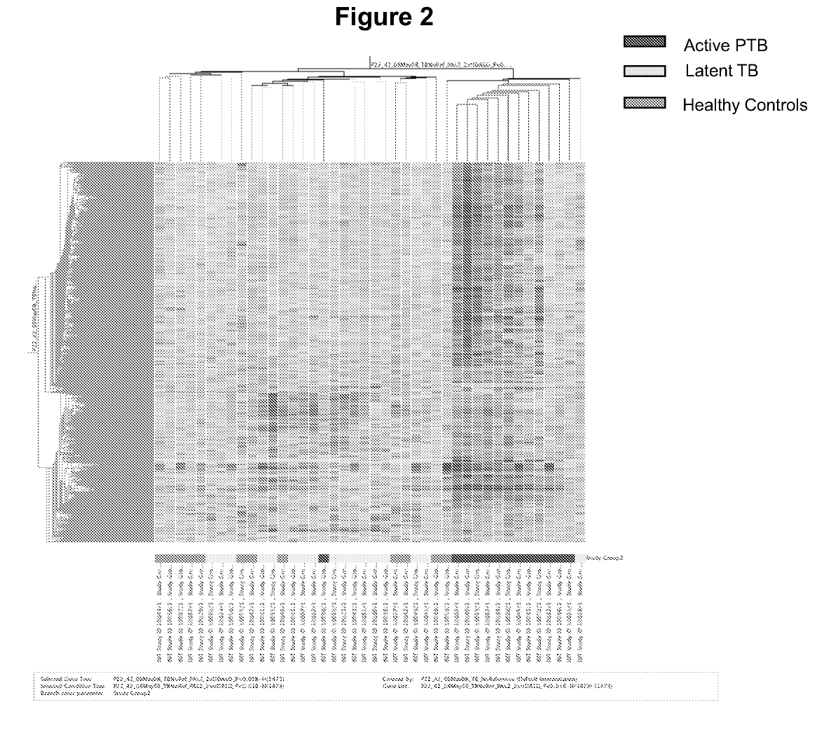

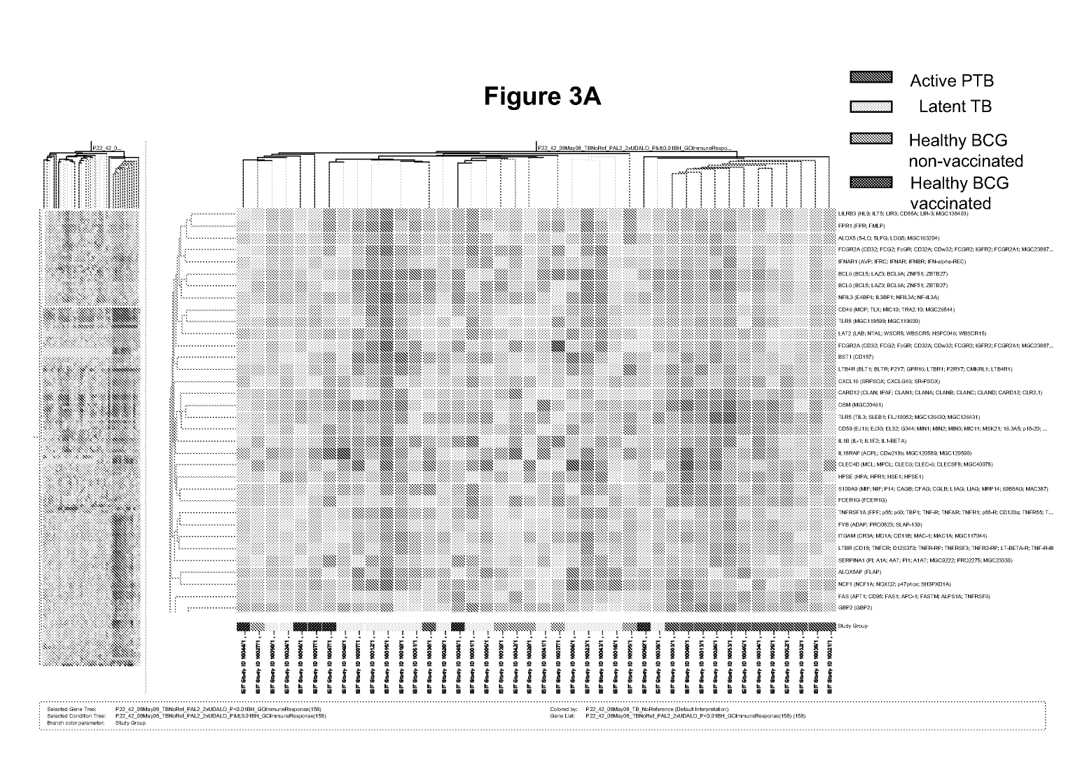

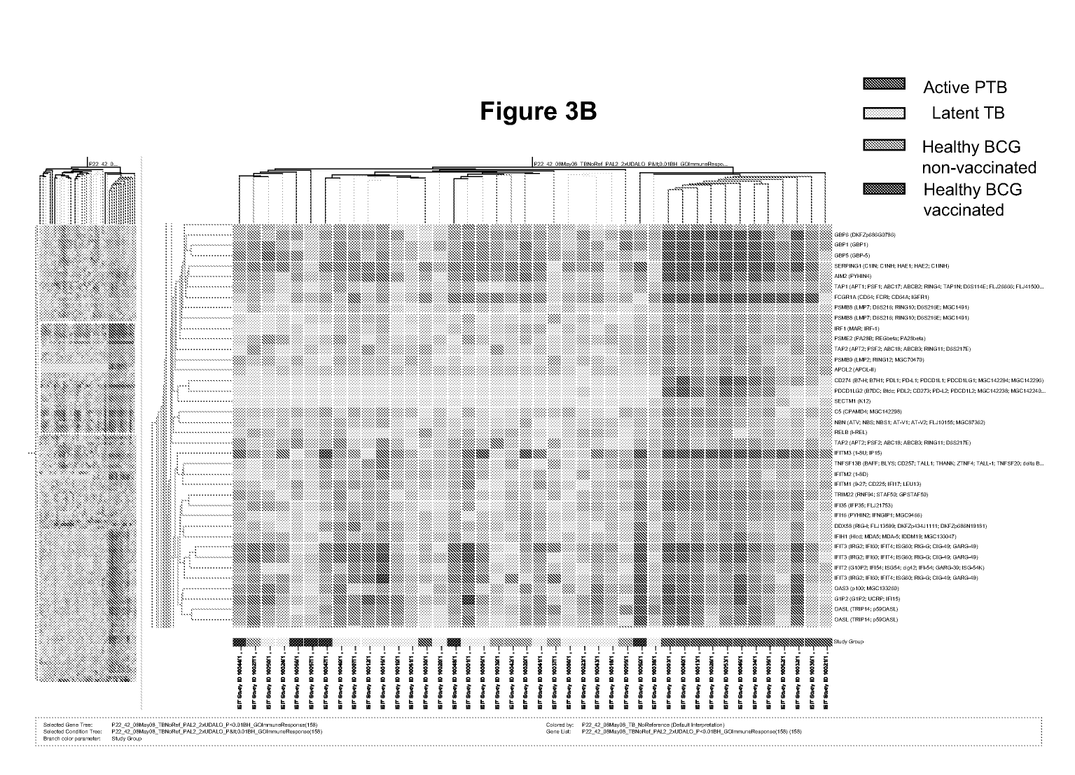

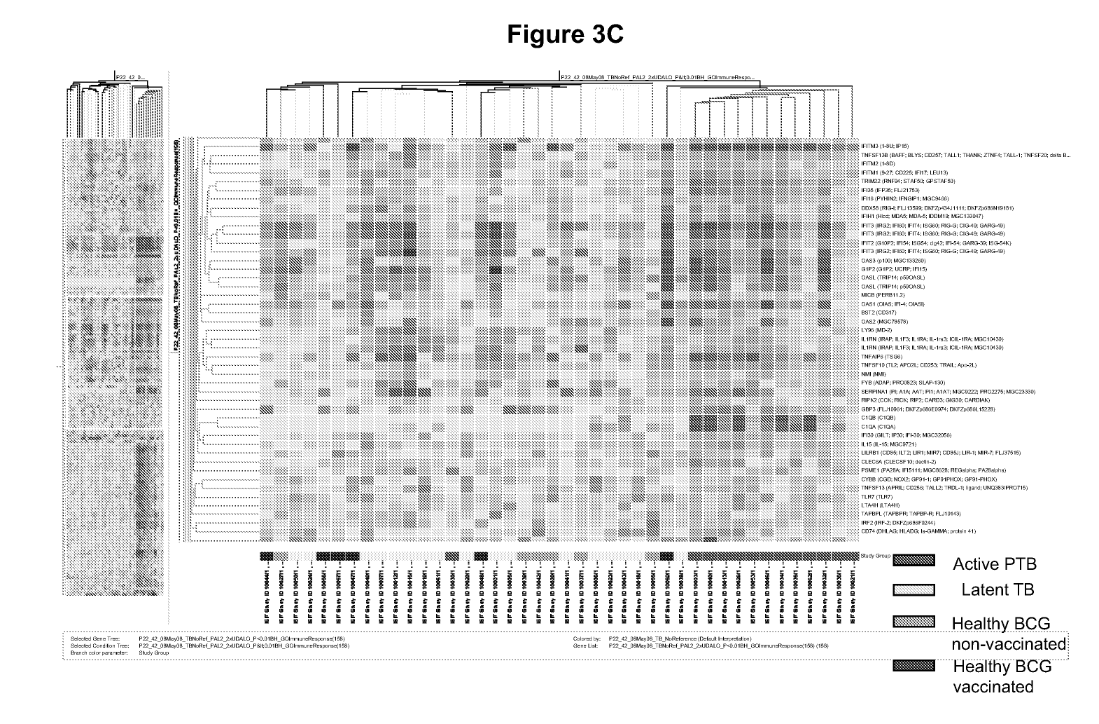

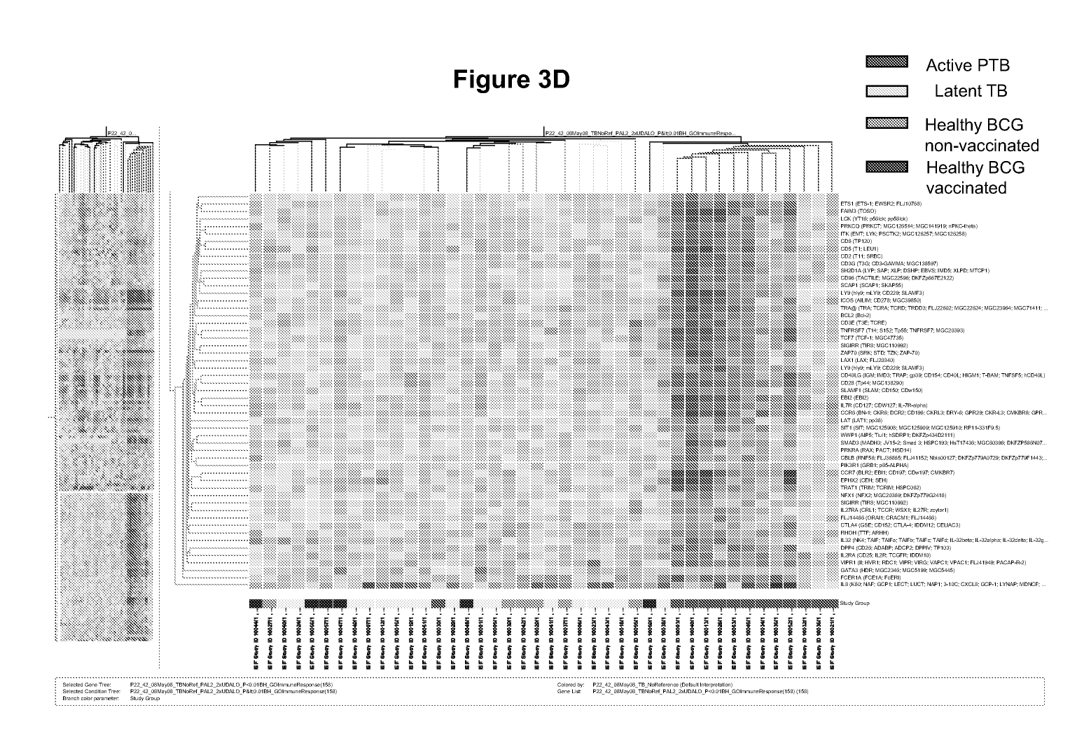

This list of 6269 genes was then further analysed using a non-parametric statistical group comparison (Kruskal-Wallis test) to identify genes that were significantly differentially expressed between groups. Using a moderately stringent multiple comparison correction for controlling Type I error (Benjamini-Hochberg correction), 1473 genes were differentially expressed/represented across the active TB and latent TB, and healthy controls (P<0.01) (FIG. 2; and listing of 1473 genes in LENGHTY TABLE, filed herewith). These clusters of genes were then correlated with relevant findings in the literature. Filtering of these genes for the ontological term “Immune response” generated a list of 158 such genes (FIGS. 3A-D; Table 2). This pattern of expression/representation of 158 genes (FIG. 3A-3D) allows discrimination of the group of Active TB patients from the Latent TB patients and from the Healthy control individuals.

TABLE 2
List of 158 genes annotated with gene ontology term biological process: immune response and
found to be significantly differentially expressed (p < 0.01) between active TB and other clinical groups.

| Gene | |
|---|---|
| Symbol | Description |
| LILRB3 | leukocyte immunoglobulin-like receptor, subfamily B (with TM and ITIM domains), member 3 |
| PGLYRP1 | peptidoglycan recognition protein 1 |
| FAS | Fas (TNF receptor superfamily, member 6) |
| IFITM3 | interferon induced transmembrane protein 3 (1-8U) |
| FCGR2A | Fc fragment of IgG, low affinity IIa, receptor (CD32) |
| FCGR2A | Fc fragment of IgG, low affinity IIa, receptor (CD32) |
| ST6GAL1 | ST6 beta-galactosamide alpha-2,6-sialyltranferase 1 |
| ETS1 | v-ets erythroblastosis virus E26 oncogene homolog 1 (avian) |
| CYBB | cytochrome b-245, beta polypeptide (chronic granulomatous disease) |
| IFNAR1 | interferon (alpha, beta and omega) receptor 1 |
| LY96 | lymphocyte antigen 96 |
| TRIM22 | tripartite motif-containing 22 |
| GBP2 | guanylate binding protein 2, interferon-inducible |
| DDX58 | DEAD (Asp-Glu-Ala-Asp) box polypeptide 58 |
| LAX1 | lymphocyte transmembrane adaptor 1 |
| IFI16 | interferon, gamma-inducible protein 16 |
| LCK | lymphocyte-specific protein tyrosine kinase |
| IL32 | interleukin 32 |
| CXCL16 | chemokine (C—X—C motif) ligand 16 |
| CD40LG | CD40 ligand (TNF superfamily, member 5, hyper-IgM syndrome) |
| TNFSF13B | tumor necrosis factor (ligand) superfamily, member 13b |
| IRF2 | interferon regulatory factor 2 |
| C5 | complement component 5 |
| CD46 | CD46 molecule, complement regulatory protein |
| TNFAIP6 | tumor necrosis factor, alpha-induced protein 6 |
| DPP4 | dipeptidyl-peptidase 4 (CD26, adenosine deaminase complexing protein 2) |
| EBI2 | Epstein-Barr virus induced gene 2 (lymphocyte-specific G protein-coupled receptor) |
| NFX1 | nuclear transcription factor, X-box binding 1 |
| MICB | MHC class I polypeptide-related sequence B |
| GBP3 | guanylate binding protein 3 |
| SLAMF7 | SLAM family member 7 |
| CARD12 | NLR family, CARD domain containing 4 |
| GBP6 | guanylate binding protein family, member 6 |
| IFIT3 | interferon-induced protein with tetratricopeptide repeats 3 |
| TAP2 | transporter 2, ATP-binding cassette, sub-family B (MDR/TAP) |
| HLA-DPB1 | major histocompatibility complex, class II, DP beta 1 |
| CD3G | CD3g molecule, gamma (CD3-TCR complex) |
| PRKCQ | protein kinase C, theta |
| IL7R | interleukin 7 receptor |
| SLAMF1 | signaling lymphocytic activation molecule family member 1 |
| CD274 | CD274 molecule |
| GBP1 | guanylate binding protein 1, interferon-inducible, 67 kDa |
| IFITM2 | interferon induced transmembrane protein 2 (1-8D) |
| ITK | IL2-inducible T-cell kinase |
| APOL2 | apolipoprotein L, 2 |
| PSME1 | proteasome (prosome, macropain) activator subunit 1 (PA28 alpha) |
| LAT2 | linker for activation of T cells family, member 2 |
| IL18RAP | interleukin 18 receptor accessory protein |
| OSM | oncostatin M |
| CD6 | CD6 molecule |
| WWP1 | WW domain containing E3 ubiquitin protein ligase 1 |
| CD3E | CD3e molecule, epsilon (CD3-TCR complex) |
| VIPR1 | vasoactive intestinal peptide receptor 1 |
| TNFSF10 | tumor necrosis factor (ligand) superfamily, member 10 |
| PRKRA | protein kinase, interferon-inducible double stranded RNA dependent activator |
| TNFRSF1A | tumor necrosis factor receptor superfamily, member 1A |
| BCL6 | B-cell CLL/lymphoma 6 (zinc finger protein 51) |
| IL8 | interleukin 8 |
| OAS3 | 2′-5′-oligoadenylate synthetase 3, 100 kDa |
| IFIH1 | interferon induced with helicase C domain 1 |
| SIGIRR | single immunoglobulin and toll-interleukin 1 receptor (TIR) domain |
| SIGIRR | single immunoglobulin and toll-interleukin 1 receptor (TIR) domain |
| SIT1 | signaling threshold regulating transmembrane adaptor 1 |
| ITGAM | integrin, alpha M (complement component 3 receptor 3 subunit) |
| C1QB | complement component 1, q subcomponent, B chain |
| IL27RA | interleukin 27 receptor, alpha |
| ALOX5AP | arachidonate 5-lipoxygenase-activating protein |
| SERPING1 | serpin peptidase inhibitor, clade G (C1 inhibitor), member 1, (angioedema, hereditary) |
| IL1RN | interleukin 1 receptor antagonist |
| IL1RN | interleukin 1 receptor antagonist |
| CLEC4D | C-type lectin domain family 4, member D |
| ICOS | inducible T-cell co-stimulator |
| OAS1 | 2′,5′-oligoadenylate synthetase 1, 40/46 kDa |
| ZAP70 | zeta-chain (TCR) associated protein kinase 70 kDa |
| IL1B | interleukin 1, beta |
| C4BPA | complement component 4 binding protein, alpha |
| TNFSF13 | tumor necrosis factor (ligand) superfamily, member 13 |
| IFI30 | interferon, gamma-inducible protein 30 |
| HPSE | heparanase |
| CD59 | CD59 molecule, complement regulatory protein |
| CTLA4 | cytotoxic T-lymphocyte-associated protein 4 |
| BCL2 | B-cell CLL/lymphoma 2 |
| TNFRSF7 | CD27 molecule |
| FPR1 | formyl peptide receptor 1 |
| IL2RA | interleukin 2 receptor, alpha |
| GATA3 | GATA binding protein 3 |
| S100A9 | 5100 calcium binding protein A9 |
| TLR8 | toll-like receptor 8 |
| NCF1 | neutrophil cytosolic factor 1, (chronic granulomatous disease, autosomal 1) |
| BCL6 | B-cell CLL/lymphoma 6 (zinc finger protein 51) |
| BST1 | bone marrow stromal cell antigen 1 |
| G1P2 | ISG15 ubiquitin-like modifier |
| C1QA | complement component 1, q subcomponent, A chain |
| TCF7 | transcription factor 7 (T-cell specific, HMG-box) |
| IFITM1 | interferon induced transmembrane protein 1 (9-27) |
| TAPBPL | TAP binding protein-like |
| AIM2 | absent in melanoma 2 |
| CCR7 | chemokine (C-C motif) receptor 7 |
| LTBR | lymphotoxin beta receptor (TNFR superfamily, member 3) |
| FYB | FYN binding protein (FYB-120/130) |
| NFIL3 | nuclear factor, interleukin 3 regulated |
| LAT | linker for activation of T cells |
| CBLB | Cas-Br-M (murine) ecotropic retroviral transforming sequence b |
| CD74 | CD74 molecule, major histocompatibility complex, class II invariant chain |
| TAP2 | transporter 2, ATP-binding cassette, sub-family B (MDR/TAP) |
| FLJ14466 | transmembrane protein 142A |
| PSMB9 | proteasome (prosome, macropain) subunit, beta type, 9 (large multifunctional peptidase 2) |
| PSMB8 | proteasome (prosome, macropain) subunit, beta type, 8 (large multifunctional peptidase 7) |
| FAIM3 | Fas apoptotic inhibitory molecule 3 |
| LTA4H | leukotriene A4 hydrolase |
| IRF1 | interferon regulatory factor 1 |
| OAS2 | 2′-5′-oligoadenylate synthetase 2, 69/71 kDa |
| RELB | v-rel reticuloendotheliosis viral oncogene homolog B, nuclear factor of kappa light |
| | polypeptide gene enhancer in B-cells 3 (avian) |
| TRA@ | T cell receptor alpha locus |
| LTB4R | leukotriene B4 receptor |
| PIK3R1 | phosphoinositide-3-kinase, regulatory subunit 1 (p85 alpha) |
| OASL | 2′-5′-oligoadenylate synthetase-like |
| OASL | 2′-5′-oligoadenylate synthetase-like |
| PSME2 | proteasome (prosome, macropain) activator subunit 2 (PA28 beta) |
| CLEC6A | C-type lectin domain family 6, member A |
| NBN | nibrin |
| FCGR1A | Fc fragment of IgG, high affinity Ia, receptor (CD64) |
| SH2D1A | SH2 domain protein 1A, Duncan's disease (lymphoproliferative syndrome) |
| IL15 | interleukin 15 |
| LY9 | lymphocyte antigen 9 |
| LILRB1 | leukocyte immunoglobulin-like receptor, subfamily B (with TM and ITIM domains), member 1 |
| APOL3 | apolipoprotein L, 3 |
| PSMB8 | proteasome (prosome, macropain) subunit, beta type, 8 (large multifunctional peptidase 7) |
| CCR6 | chemokine (C-C motif) receptor 6 |
| PDCD1LG2 | programmed cell death 1 ligand 2 |
| CD96 | CD96 molecule |
| EPHX2 | epoxide hydrolase 2, cytoplasmic |
| BST2 | bone marrow stromal cell antigen 2 |
| RIPK2 | receptor-interacting serine-threonine kinase 2 |
| SCAP1 | src kinase associated phosphoprotein 1 |
| GBP5 | guanylate binding protein 5 |
| TRAT1 | T cell receptor associated transmembrane adaptor 1 |
| ALOX5 | arachidonate 5-lipoxygenase |
| LY9 | lymphocyte antigen 9 |
| TAP1 | transporter 1, ATP-binding cassette, sub-family B (MDR/TAP) |
| RHOH | ras homolog gene family, member H |
| IFI35 | interferon-induced protein 35 |
| CD28 | CD28 molecule |
| FYB | FYN binding protein (FYB-120/130) |
| IFIT2 | interferon-induced protein with tetratricopeptide repeats 2 |
| TLR7 | toll-like receptor 7 |
| CD2 | CD2 molecule |
| FCER1G | Fc fragment of IgE, high affinity I, receptor for; gamma polypeptide |
| SMAD3 | SMAD family member 3 |
| FCER1A | Fc fragment of IgE, high affinity I, receptor for; alpha polypeptide |
| SERPINA1 | serpin peptidase inhibitor, clade A (alpha-1 antiproteinase, antitrypsin), member 1 |
| SERPINA1 | serpin peptidase inhibitor, clade A (alpha-1 antiproteinase, antitrypsin), member 1 |
| SECTM1 | secreted and transmembrane 1 |
| NMI | N-myc (and STAT) interactor |
| TLR5 | toll-like receptor 5 |
| IFIT3 | interferon-induced protein with tetratricopeptide repeats 3 |
| IFIT3 | interferon-induced protein with tetratricopeptide repeats 3 |
| CD5 | CD5 molecule |

Genes over-expressed/represented in active TB: Of interest is that a large number of IFN-associated/inducible genes were expressed: for example interferon (IFN)-inducible genes, e.g., SOCS1, STAT1, PML (TRIM19), TRIM22, many guanylate binding proteins, and many other IFN-inducible genes as indicated in Table 2, as expected in active TB, but interestingly these were not evident in latent TB patients, although these patients representation/expression of IFN-γ transcripts in whole blood was in fact higher than the active TB patients. To focus in on this, certain families of genes, some of which are known to be upregulated by IFNs and others not, were further studied, including the TRIM family.

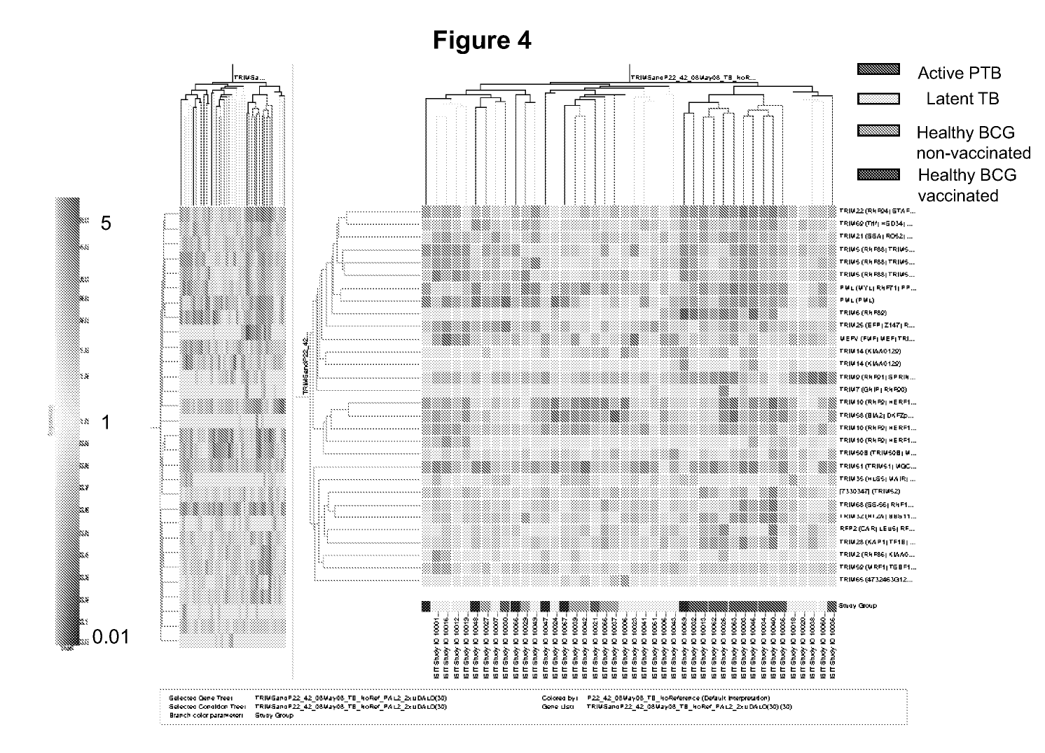

A subset of TRIMS are over-expressed/represented in Active TB: The tripartite motif (TRIM) family of proteins are characterized by a discreet structure (Reymond, A., EMBO J., 2001) and have been shown to have multiple functions, including E3 ubiquitin ligases activity, induction of cellular proliferation, differentiation and apoptosis, immune cell signalling (Meroni, G., Bioessays, 2005). Their involvement has been implicated in protein-protein interactions, autoimmunity and development (Meroni, G., Bioessays, 2005). Furthermore, a number of TRIM proteins have been found to have anti-viral activity and are possibly involved in innate immunity (Nisole, F, 2005, Nat. Rev. Microbiol.; Gack, M U., 2007, Nature). Interestingly, 30 TRIM transcripts (some overlapping probes) were shown to be expressed in active TB, with some also expressed in latent TB and healthy control blood (FIG. 4; Table 3). The majority of these TRIMs have been previously shown to be expressed in both human macrophages and mouse macrophages and dendritic cells (Rajsbaum, 2008, EJI; Martinez, F O., J. Imm., 2006) and regulated by IFNs, whereas TRIMs shown to be constitutively expressed in DC or in T cells (Rajsbaum, 2008, EJI) were not detected or were not found to be differentially expressed in active or latent TB versus healthy control blood. Interestingly, it was found that TRIM 5, 6, 19(PML), 21, 22, 25, 68 are overrepresented/expressed; while the others are underepreresented/expressed: TRIM 28, 32, 51, 52, 68. Of interest a group of TRIMs was highly expressed in active TB, but low to undetectable in latent TB and healthy controls, and four of these (TRIM 5, 6, 21, 22) have been show to cluster on human chromosome 11, and reported to have anti-viral activity (Song, B., 2005, J. Virol.); Li, X, Virology, 2007). A group of TRIMs however, were found to be under-expressed in the blood of active TB patients versus that of latent TB and healthy controls, including TRIM 28, 32, 51, 52 68, and these have been reported to either not be expressed in human blood-derived macrophages (TRIM 51) or only expressed in undifferentiated monocytes (TRIM-28, 52) or non-activated macrophages or alternately activated macrophages (TRIM-32), or only upregulated to a low level in activated macrophages differentiated from human blood (TRIM-68) (Martinez, F O., J. Imm., 2006).

TABLE 3
TRIM genes differentially expressed in active pulmonary tuberculosis,
latent tuberculosis and healthy controls.

| | Gene | |
|---|---|---|
| Common Name | Symbol | Description |
| RNF94; STAF50; | TRIM22 | tripartite motif-containing 22 |
| GPSTAF50 | | |
| RNF91; SPRING; | TRIM9 | tripartite motif-containing 9 |
| KIAA0282 | | |
| MYL; RNF71; PP8675; | PML | promyelocytic leukemia |
| TRIM19 | | |
| RNF89 | TRIM6 | tripartite motif-containing 6 |
| TRIM51; MGC10977 | TRIM51 | SPRY domain containing 5 |
| RNF9; HERF1; RFB30; | TRIM10 | tripartite motif-containing 10 |
| MGC141979 | | |
| PML | PML | promyelocytic leukemia; |
| | | synonyms: MYL, RNF71, PP8675, |
| | | TRIM19; isoform 7 is encoded by |
| | | transcript variant 7; promyelocytic |
| | | leukemia, inducer of; tripartite |
| | | motif protein TRIM19; |
| | | promyelocytic leukemia protein; |
| | | Homo sapiens promyelocytic |
| | | leukemia (PML), transcript |
| | | variant 7, mRNA. |
| RNF88; TRIM5alpha | TRIM5 | tripartite motif-containing 5 |
| RNF88; TRIM5alpha | TRIM5 | tripartite motif-containing 5 |
| BIA2; DKFZp434C091 | TRIM58 | tripartite motif-containing 58 |
| Trif; HSD34; RNF36 | TRIM69 | tripartite motif-containing 69 |
| RNF88; TRIM5alpha | TRIM5 | tripartite motif-containing 5 |
| SSA; RO52; SSA1; | TRIM21 | tripartite motif-containing 21 |
| RNF81 | | |
| KIAA0129 | TRIM14 | tripartite motif-containing 14 |
| RNF9; HERF1; RFB30; | TRIM10 | tripartite motif-containing 10 |
| MGC141979 | | |
| EFP; Z147; RNF147; | TRIM25 | tripartite motif-containing 25 |
| ZNF147 | | |
| HLS5; MAIR; | TRIM35 | tripartite motif-containing 35 |
| KIAA1098; MGC17233 | | |
| RNF86; KIAA0517 | TRIM2 | tripartite motif-containing 2 |
| RNF9; HERF1; RFB30; | TRIM10 | tripartite motif-containing 10 |
| MGC141979 | | |
| GNIP; RNF90 | TRIM7 | tripartite motif-containing 7 |
| KIAA0129 | TRIM14 | tripartite motif-containing 14 |
| TRIM50B; MGC45477 | TRIM50B | tripartite motif-containing 73 |
| 4732463G12Rik | TRIM65 | tripartite motif-containing 65 |
| MRF1; TSBF1; RNF104; | TRIM59 | tripartite motif-containing 59 |
| TRIM57; MGC26631; | | |
| MGC129860; | | |
| MGC129861 | | |
| FMF; MEF; TRIM20; | MEFV | Mediterranean fever |
| MGC126560; | | |
| MGC126586 | | |
| TRIM52 | | Tripartite motif-containing 52 |
| CAR; LEU5; RFP2; | RFP2 | tripartite motif-containing 13 |
| DLEU5; RNF77 | | |
| KAP1; TF1B; RNF96; | TRIM28 | tripartite motif-containing 28 |
| TIF1B; FLJ29029 | | |
| SS-56; RNF137; | TRIM68 | tripartite motif-containing 68 |
| FLJ10369; MGC126176 | | |
| HT2A; BBS11; TATIP; | TRIM32 | tripartite motif-containing 32 |
| LGMD2H | | |

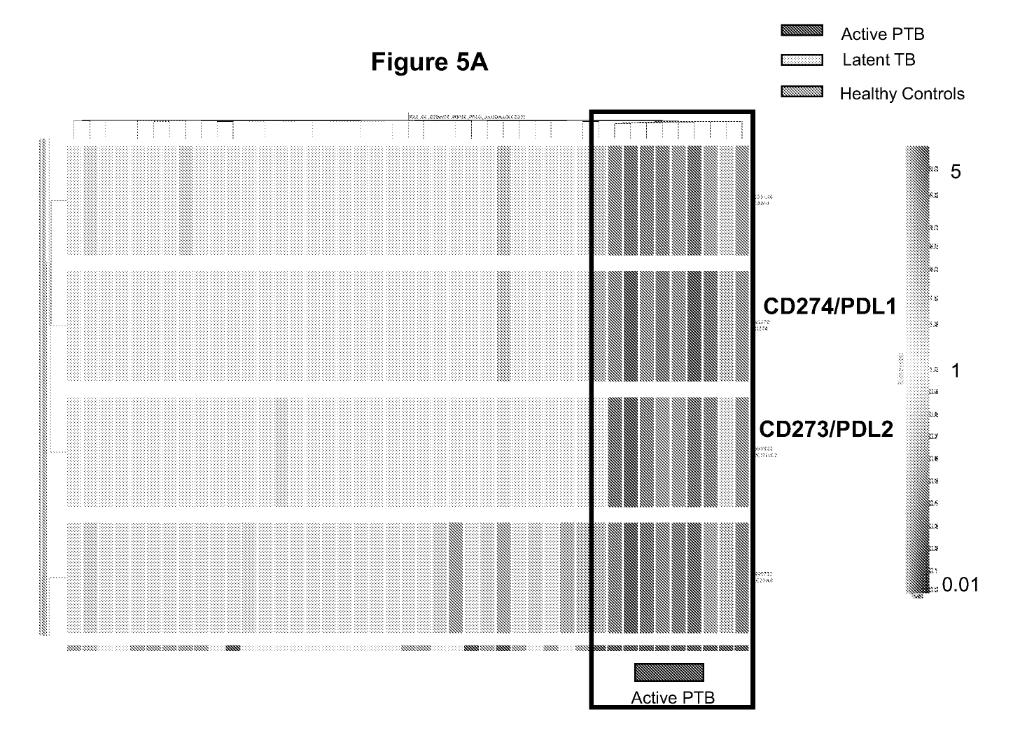

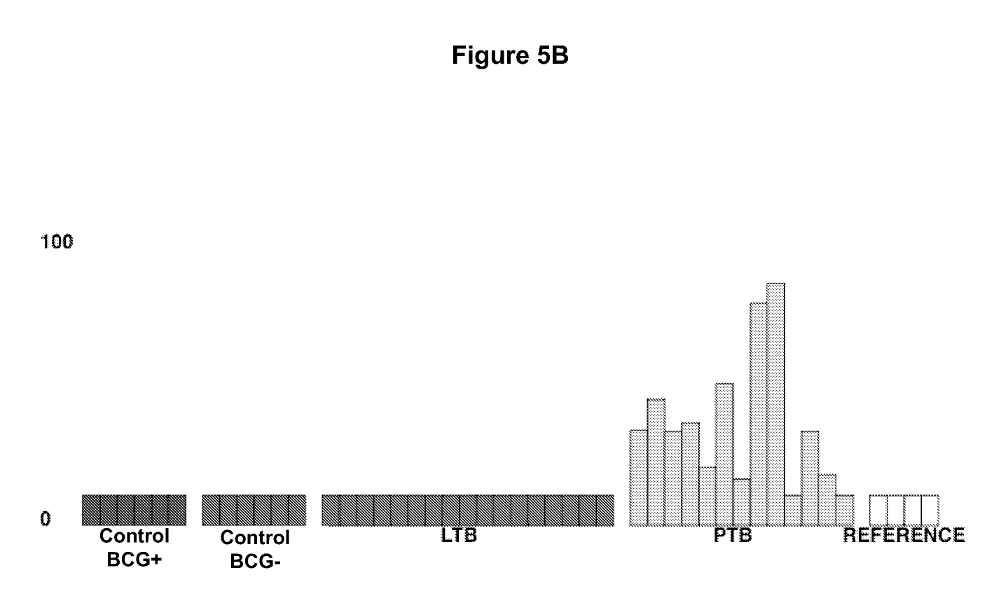

Selective over-expression/representation of specific immunomodulatory ligands in Active TB Patients: Analysis of the distinct transcriptional profiles revealed that transcripts from the genes CD274 (PDL1) and PCDLG2 (PDL2, CD273) are expressed only in the active TB patients (FIGS. 5A and B). These molecules have been previously shown to be involved in the regulation of the immune response to both acute and chronic viral infection (A Sharpe, Ann. Rev. Imm.). These molecules act as inhibitory co-stimulatory receptors for the molecule PD1 in interactions between T cells and APCs, and blockade of this pathway has been shown to restore the proliferative and effector functions of antigen specific T cells in HIV, Hepatitis B and C infection.

Genes under-expressed/represented in active TB: Strikingly, a number of genes known to be expressed in T cells (some also on NK and B cells), were found to be profoundly down-regulated/under-represented in the blood of active TB patients (FIG. 3D), (but not in latent TB or healthy controls, including, CD3, CTLA-4, CD28, ZAP-70 (T, NK and B cells), IL-7R, CD2 (also on B cells), SLAM (also on NK cells), CCR7, GATA-3 (also in NK cells). This could indicate that gene expression was down-regulated in T, NK and B cells during active PTB, or that the cells had been recruited elsewhere (e.g., the lung) as a result of infection with M. tuberculosis. This is currently under investigation using flow cytometric analysis of blood from the different patient groups, as well as by transcriptional analysis of purified populations of T cells from the different patient groups.

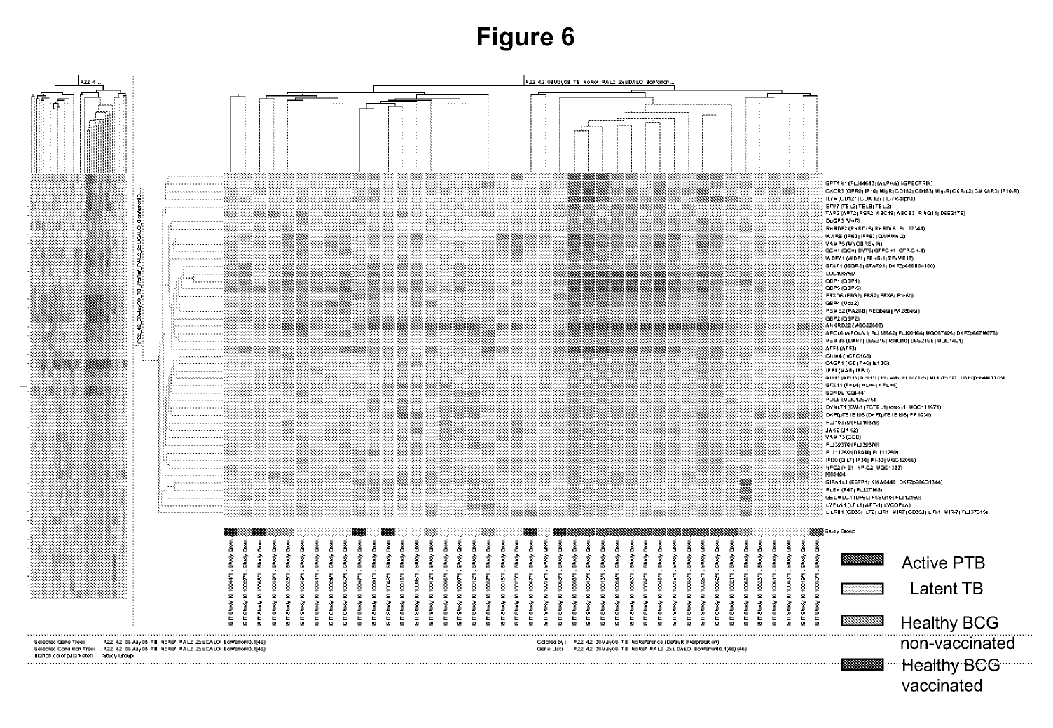

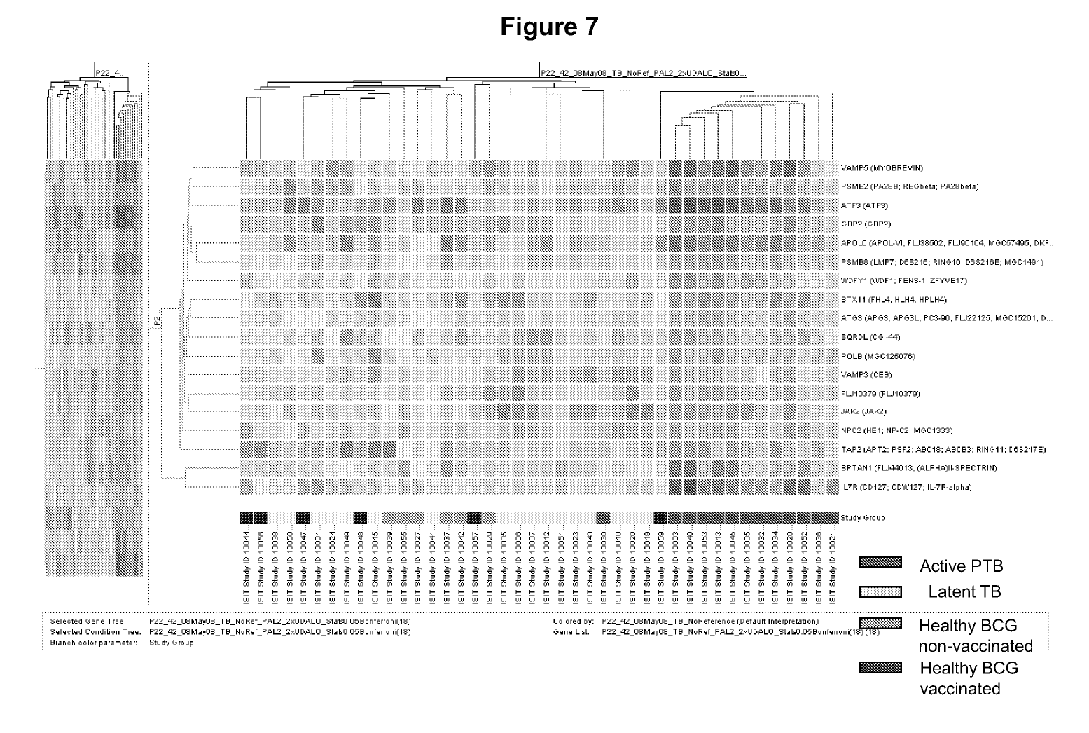

Higher Stringency Statistical analysis of transcriptional profiles in latent and active TB patients versus healthy controls. Statistical group comparison was further performed as before by identifying differentially expressed genes between the groups using the non-parametric Kruskal-Wallis test, but now using the most stringent multiple comparison correction for controlling Type I error (Bonferroni correction). With this increased stringency 46 genes (P<0.1) and 18 genes (P<0.05) were identified as differentially expressed between groups (FIGS. 6 and 7; Tables 4 and 5). Of the 46 genes a large number of IFN-inducible genes, such as STAT-1, GBP and IRF-1 were still observed to be over-expressed/represented in the blood from active TB patients, and either down-regulated or unchanged in the latent patients or healthy controls. A number of these genes were also found to be over-expressed/represented in the blood of active TB patients, even with the highest stringency analysis which still extracted genes (Bonferroni correction, P<0.05). Only 3 transcripts in active TB were still observed to be down-regulated/under-represented within the 46 gene group, including IL-7R (expressed in T cells), the chemokine receptor CXCR3 (lost at higher statistical stringency) and alpha II-spectrin. The underexpression/representation of CXCR3 is of interest since this chemokine receptor has been shown to be highly expressed in Th1 cells required for protection against mycobacterial infection, which may reflect their suppression or migration out of blood to infected tissue. Table 5 includes 18 genes, with IL7R and SPTAN1 being underrepresented/expressed in active PTB, and all others being overrepresented/expressed and diagnostic for active disease.

TABLE 4
Genes significantly differentially expressed between active TB and other
clinical groups.

| Gene Symbol | Description |
|---|---|
| FAM84B | family with sequence similarity 84, member B |
| CXCR3 | chemokine (C—X—C motif) receptor 3 |
| ETV7 | ets variant gene 7 (TEL2 oncogene) |
| DUSP3 | dual specificity phosphatase 3 (vaccinia virus |
| | phosphatase VH1-related) |
| WARS | tryptophanyl-tRNA synthetase |
| CNIH4 | cornichon homolog 4 (Drosophila) |
| STAT1 | signal transducer and activator of transcription 1, |
| | 91 kDa |
| IRF1 | interferon regulatory factor 1 |
| LILRB1 | leukocyte immunoglobulin-like receptor, subfamily B |
| | (with TM and ITIM domains), member 1 |
| SIPA1L1 | signal-induced proliferation-associated 1 like 1 |
| GSDMDC1 | gasdermin domain containing 1 |
| DYNLT1 | dynein, light chain, Tctex-type 1 |
| DKFZp761E198 | DKFZp761E198 protein |
| LOC400759 | |
| GBP1 | guanylate binding protein 1, interferon-inducible, |
| | 67 kDa |
| GBP5 | guanylate binding protein 5 |
| FLJ11259 | damage-regulated autophagy modulator |
| LYPLA1 | lysophospholipase I |
| RHBDF2 | rhomboid 5 homolog 2 (Drosophila) |
| PLEK | pleckstrin |
| ANKRD22 | ankyrin repeat domain 22 |
| CASP1 | caspase 1, apoptosis-related cysteine peptidase |
| | (interleukin 1, beta, convertase) |
| FLJ39370 | chromosome 4 open reading frame 32 |
| FBXO6 | F-box protein 6 |
| GCH1 | GTP cyclohydrolase 1 (dopa-responsive dystonia) |
| GBP4 | guanylate binding protein 4 |
| IFI30 | interferon, gamma-inducible protein 30 |
| VAMP5 | vesicle-associated membrane protein 5 (myobrevin) |
| GBP2 | guanylate binding protein 2, interferon-inducible |
| STX11 | syntaxin 11 |
| SPTAN1 | spectrin, alpha, non-erythrocytic 1 (alpha-fodrin) |
| POLB | polymerase (DNA directed), beta |
| IL7R | interleukin 7 receptor |
| APOL6 | apolipoprotein L, 6 |
| ATG3 | ATG3 autophagy related 3 homolog (S. cerevisiae) |
| SQRDL | sulfide quinone reductase-like (yeast) |
| PSME2 | proteasome (prosome, macropain) activator subunit 2 |
| | (PA28 beta) |
| FLJ10379 | S1 RNA binding domain 1 |
| WDFY1 | WD repeat and FYVE domain containing 1 |
| TAP2 | transporter 2, ATP-binding cassette, sub-family B |
| | (MDR/TAP) |
| NPC2 | Niemann-Pick disease, type C2 |
| ATF3 | activating transcription factor 3 |
| VAMP3 | vesicle-associated membrane protein 3 (cellubrevin) |
| PSMB8 | proteasome (prosome, macropain) subunit, beta type, |
| | 8 (large multifunctional peptidase7) |
| JAK2 | Janus kinase 2 (a protein tyrosine kinase) |

TABLE 5
18 genes significantly differentially expressed between active TB and
other clinical groups.

| Gene Symbol | Description |
|---|---|
| VAMP5 | vesicle-associated membrane protein 5 (myobrevin) |
| GBP2 | guanylate binding protein 2, interferon-inducible |
| STX11 | syntaxin 11 |
| SPTAN1 | spectrin, alpha, non-erythrocytic 1 (alpha-fodrin) |
| POLB | polymerase (DNA directed), beta |
| IL7R | interleukin 7 receptor |
| APOL6 | apolipoprotein L, 6 |
| ATG3 | ATG3 autophagy related 3 homolog (S. cerevisiae) |
| SQRDL | sulfide quinone reductase-like (yeast) |
| PSME2 | proteasome (prosome, macropain) activator subunit 2 |
| | (PA28 beta) |
| FLJ10379 | S1 RNA binding domain 1 |
| WDFY1 | WD repeat and FYVE domain containing 1 |
| TAP2 | transporter 2, ATP-binding cassette, sub-family B |
| | (MDR/TAP) |
| NPC2 | Niemann-Pick disease, type C2 |
| ATF3 | activating transcription factor 3 |
| VAMP3 | vesicle-associated membrane protein 3 (cellubrevin) |
| PSMB8 | proteasome (prosome, macropain) subunit, beta type, 8 |
| | (large multifunctional peptidase7) |
| JAK2 | Janus kinase 2 (a protein tyrosine kinase) |

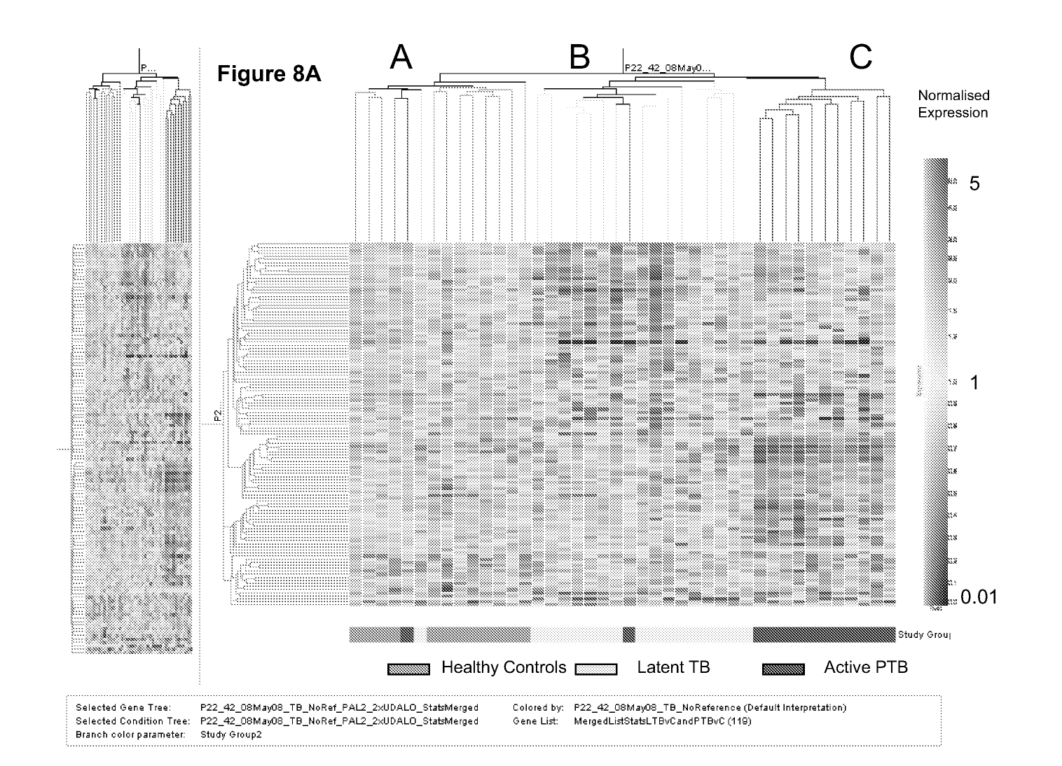

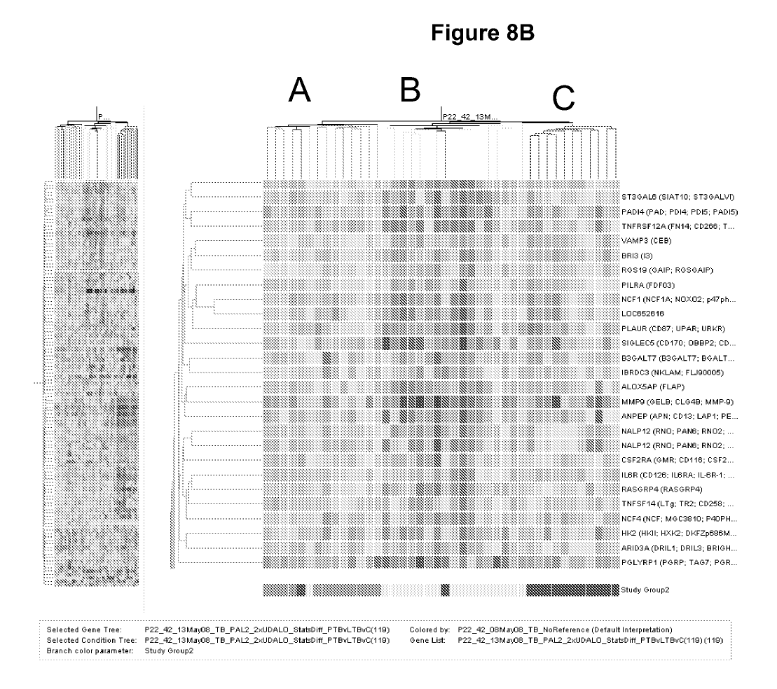

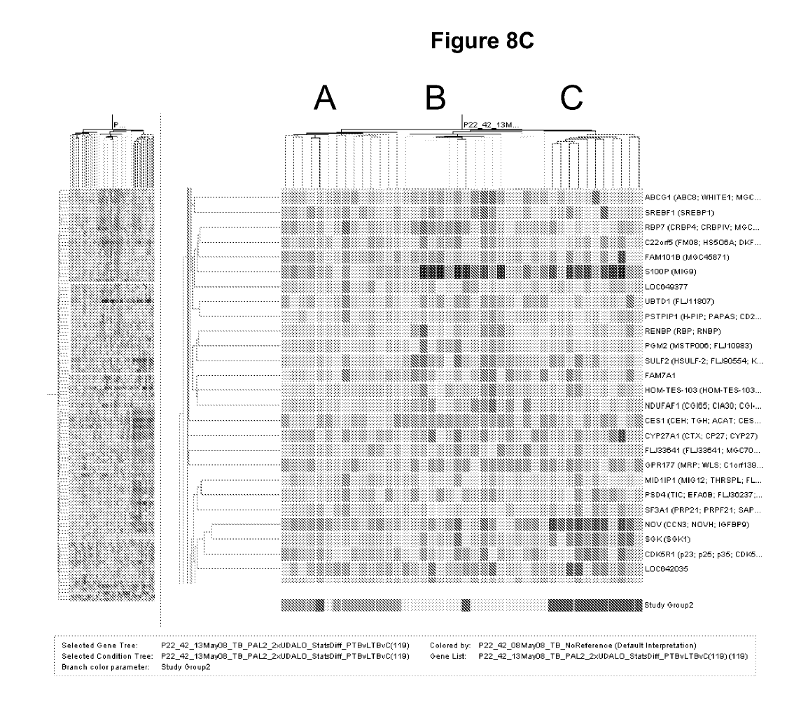

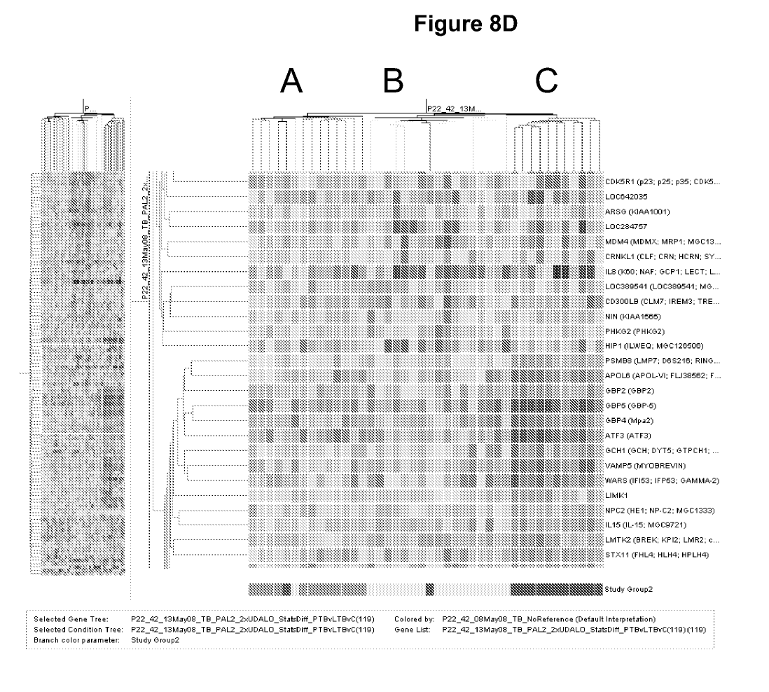

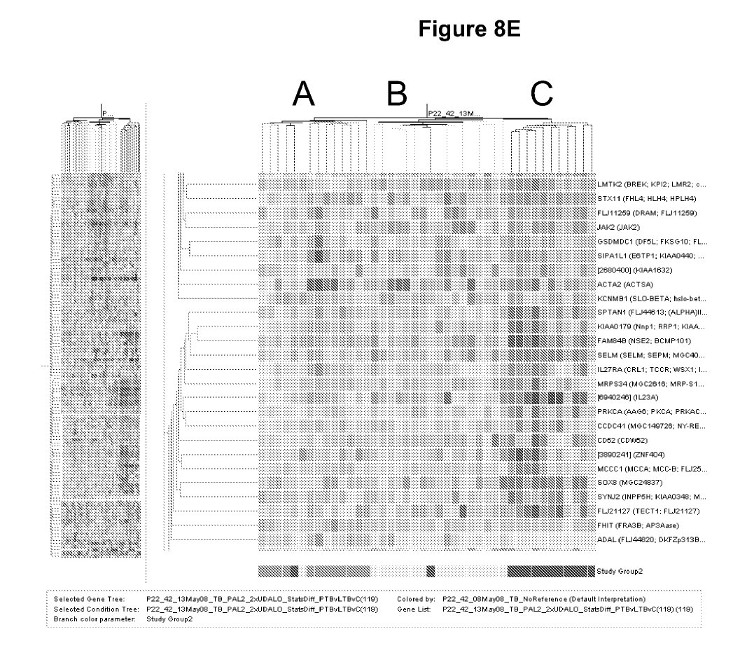

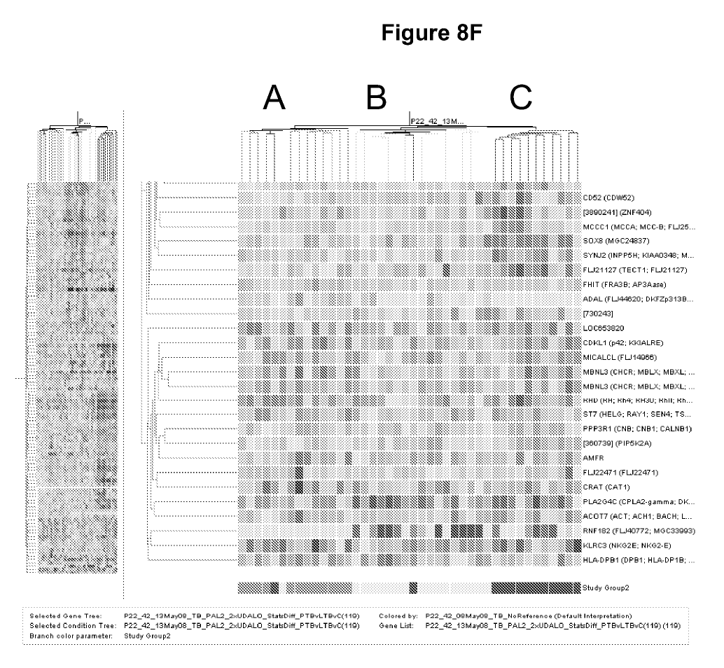

Improved discrimination between patients with active and latent TB and healthy controls: The approaches described above although able to discriminate active TB from latent TB and healthy controls are less able to discriminate between all three clinical groups. To select discriminating genes the following approach was used. First, genes expressed in blood from healthy individuals were compared versus latent TB patients, using the Wilcoxon-Mann-Whitney test at a p<0.005, which yielded 89 discriminatory genes. Genes expressed in blood from healthy individuals versus active TB patients were then compared, again using the Wilcoxon-Mann-Whitney test but with a p<0.5, and the most stringent Bonferroni correction factor, which yielded a list of 30 discriminatory genes. This list was combined to give a total list of 119 discriminating genes (Table 6). This list of genes was then used to interrogate the dataset of all clinical groups using unsupervised clustering analysis by Pearson correlation. This analysis generated three distinct clusters of clinical groups (FIGS. 8A to 8F): one cluster is composed of 11 out of 13 of the active TB patients (FIG. 8, Cluster C); a second cluster is composed of 16 out of 17 latent TB patients, and 1 active TB patient (FIG. 8, Cluster B); a third cluster contains all 12 healthy controls included in the study, plus 1 active TB and 1 latent TB outlier (FIG. 8, Cluster A). For each of FIGS. 8A to 8F, clusters of patients/clinical groups are presented horizontally and clusters of genes are presented vertically. This pattern of expression/representation of the whole list of 119 genes (FIG. 8A) now allows discrimination of all three clinical groups from each other: i.e., allows discrimination of Active TB, Latent TB and Healthy individuals from each other, each clinical group exhibiting a unique pattern of expression/representation of these 119 genes or subgroups thereof. The skilled artisan will recognize that 1, 2, 3, 4, 5, 6, 7, 8, 10, 12, 15, 20, 25, 30, 35 or more genes may be placed in a dataset that represents a cluster of genes that may be compared across clusters of clinical groups A (Healthy), B (Latent), C (Active), and that either alone or in combination with other such clusters, each clinical group can exhibit a unique pattern of expression/representation obtained from these 119 genes.

Specifically, FIG. 8B demonstrates that the genes ST3GAL6, PAD14, TNFRSF12A, VAMP3, BR13, RGS19, PILRA, NCF1, LOC652616, PLAUR(CD87), SIGLEC5, B3GALT7, IBRDC3(NKLAM), ALOX5AP(FLAP), MMP9, ANPEP(APN), NALP12, CSF2RA, IL6R(CD126), RASGRP4, TNFSF14(CD258), NCF4, HK2, ARID3A, PGLYRP1(PGRP) are underexpressed/underrepresented in the blood of Latent TB patients but not in the blood of Healthy individuals or of Active TB patients.

The genes presented in FIG. 8C, ABCG1, SREBF1, RBP7(CRBP4), C22orf5, FAM101B, S100P, LOC649377, UBTD1, PSTPIP-1, RENBP, PGM2, SULF2, FAM7A1, HOM-TES-103, NDUFAF1, CES1, CYP27A1, FLJ33641, GPR177, MID1IP1(MIG-12), PSD4, SF3A1, NOV(CCN3), SGK(SGK1), CDK5R1, LOC642035, are shown to be overexpressed/overrepresented in the blood of Healthy control individuals but were underexpressed/underrepresented in the blood of Latent TB patients, and to a great extent were underexpressed/underrepresented in the blood of Active TB patients.

The pattern of genes in FIG. 8D, ARSG, LOC284757, MDM4, CRNKL1, IL8, LOC389541, CD300LB, NIN, PHKG2, HIP1, were shown to be overexpressed/overrepresented in the blood of Healthy individuals but were underexpressed/underrepresented in the blood of both Latent and Active TB patients. Conversely, the genes in FIG. 8D, PSMB8(LMP7), APOL6, GBP2, GBP5, GBP4, ATF3, GCH1, VAMP5, WARS, LIMK1, NPC2, IL-15, LMTK2, STX11(FHL4), were shown to be overexpressed/overrepresented in the blood of Active TB, but underexpressed/underrepresented in the blood of Latent TB patients and Healthy control individuals.

The pattern of genes in FIG. 8E, of FLJ11259(DRAM), JAK2, GSDMDC1(DF5L)(FKSG10), SIPAIL1, [2680400](KIAA1632), ACTA2(ACTSA), KCNMB1(SLO-BETA), were all overexpressed/overrepresented in blood from Active TB patients but not represented or even underexpressed/underrepresented in the blood from Latent TB patients and Healthy control individuals. Conversely, the genes SPTANI, KIAAD179(Nnp1)(RRP1), FAM84B(NSE2), SELM, IL27RA, MRPS34, [6940246](IL23A), PRKCA(PKCA), CCDC41, CD52(CDW52), [3890241](ZN404), MCCC1(MCCA/B), SOX8, SYNJ2, FLJ21127, FHIT, were underexpressed/underrepresented in the blood of Active TB patients but not in the blood of Latent TB patients or Healthy Control individuals, where they were overexpressed/overrepresented.

Many of the genes (within these 119 genes selected by this method described above) found to be overexpressed/overrepresented in the blood of Active TB patients listed in FIGS. 8D and 8E, were common to those identified by the alternative method using Higher Stringency Analysis of transcriptional profiles in active, latent TB patients and healthy controls described earlier (genes shown as underlined above from FIGS. 8D and 8E are contained in list of genes in FIG. 7, Table 5, 18 genes p<0.05; genes shown as italicised above from FIGS. 8D and 8E are contained in list of genes in FIG. 6, Table 4, 46 genes P<0.1).

The pattern of genes shown in FIG. 8F, CD52(CDW52), [3890241](ZNF404), MCCC1(MCCA/B), SOX8, SYNJ2, FLJ21127, FHIT, were underexpressed/underrepresented in the blood of Active TB patients but not in the blood of Latent TB patients or Healthy Control individuals, where they were if anything overexpressed/overrepresented. This is also presented (overlap) in FIG. 8E. Genes CDKL1(p42), MICALCL, MBNL3, RHD, ST7(RAY1), PPR3R1, [360739](PIP5K2A), AMFR, FLJ22471, CRAT(CAT1), PLA2G4C, ACOT7(ACT)(ACH1), RNF182, KLRC3(NKG2E), HLA-DPB 1, were underexpressed/underrepresented in the blood of Healthy Control individuals, but were overexpressed/overrepresented in the blood of the Latent TB patients, and overexpressed/overrepresented in the blood of most Active TB patients (FIG. 8F). To conclude, the aggregate pattern of expression of the total of 119 genes in FIG. 8A (broken down for legibility of genes and specificity between clinical states in FIGS. 8B-8F) that distinguishes between infected (Active TB and Latent TB) patients from non-infected patients (Healthy Controls) and additionally, distinguishes between the two groups of infected patients, that is Active and Latent TB patients. Many of the genes overexpressed in the blood of active TB patients via this method were the same genes as those identified using the strictest statistical filtering (shown in FIG. 7, Table 6), and many were IFN-inducible and/or involved in endocytic cellular traffic and/or lipid metabolism.

TABLE 6
Genes found to be significantly differentially expressed between latent and healthy or
between active and healthy, which when used in combination differentiate between active, healthy
and latent using unsupervised pearson correlation clustering algorithms (119 genes).

| Gene Symbol | Description |
|---|---|
| HMFN0839 | lung cancer metastasis-associated protein |
| LOC653820 | |
| MID1IP1 | MID1 interacting protein 1 (gastrulation specific G12 homolog (zebrafish)) |
| SPTAN1 | spectrin, alpha, non-erythrocytic 1 (alpha-fodrin) |
| NALP12 | NLR family, pyrin domain containing 12 |
| PSMB8 | proteasome (prosome, macropain) subunit, beta type, 8 (large multifunctional peptidase 7) |
| RNF182 | ring finger protein 182 |
| KCNMB1 | potassium large conductance calcium-activated channel, subfamily M, beta member 1 |
| | Interleukin 23, alpha subunit p19 |
| CDKL1 | cyclin-dependent kinase-like 1 (CDC2-related kinase) |
| IL8 | interleukin 8 |
| NOV | nephroblastoma overexpressed gene |
| APOL6 | apolipoprotein L, 6 |
| KLRC3 | killer cell lectin-like receptor subfamily C, member 3 |
| SOX8 | SRY (sex determining region Y)-box 8 |
| B3GALT7 | UDP-GlcNAc:betaGal beta-1,3-N-acetylglucosaminyltransferase 8 |
| GCH1 | GTP cyclohydrolase 1 (dopa-responsive dystonia) |
| IL6R | interleukin 6 receptor |
| RASGRP4 | RAS guanyl releasing protein 4 |
| SGK | serum/glucocorticoid regulated kinase |
| LOC389541 | similar to CG14977-PA |
| MICALCL | MICAL C-terminal like |
| VAMP3 | vesicle-associated membrane protein 3 (cellubrevin) |
| NPC2 | Niemann-Pick disease, type C2 |
| SYNJ2 | synaptojanin 2 |
| NIN | ninein (GSK3B interacting protein) |
| MBNL3 | muscleblind-like 3 (Drosophila) |
| FLJ11259 | damage-regulated autophagy modulator |
| NALP12 | NLR family, pyrin domain containing 12 |
| LIMK1 | |
| ARSG | arylsulfatase G |
| FLJ33641 | chromosome 5 open reading frame 29 |
| PADI4 | peptidyl arginine deiminase, type IV |
| RENBP | renin binding protein |
| SULF2 | sulfatase 2 |
| GSDMDC1 | gasdermin domain containing 1 |
| ST7 | suppression of tumorigenicity 7 |
| RBP7 | retinol binding protein 7, cellular |
| HK2 | hexokinase 2 |
| VAMP5 | vesicle-associated membrane protein 5 (myobrevin) |
| GPR177 | G protein-coupled receptor 177 |
| CES1 | carboxylesterase 1 (monocyte/macrophage serine esterase 1) |
| CD52 | CD52 molecule |
| ABCG1 | ATP-binding cassette, sub-family G (WHITE), member 1 |
| GBP5 | guanylate binding protein 5 |
| MDM4 | Mdm4, transformed 3T3 cell double minute 4, p53 binding protein (mouse) |
| SIGLEC5 | sialic acid binding Ig-like lectin 5 |
| ARID3A | AT rich interactive domain 3A (BRIGHT-like) |
| KIAA0179 | ribosomal RNA processing 1 homolog B (S. cerevisiae) |
| PSD4 | pleckstrin and Sec7 domain containing 4 |
| ALOX5AP | arachidonate 5-lipoxygenase-activating protein |
| CSF2RA | colony stimulating factor 2 receptor, alpha, low-affinity (granulocyte-macrophage) |
| MMP9 | matrix metallopeptidase 9 (gelatinase B, 92 kDa gelatinase, 92 kDa type IV collagenase) |
| PGLYRP1 | peptidoglycan recognition protein 1 |
| CYP27A1 | cytochrome P450, family 27, subfamily A, polypeptide 1 |
| LMTK2 | lemur tyrosine kinase 2 |
| BRI3 | brain protein I3 |
| PILRA | paired immunoglobin-like type 2 receptor alpha |
| | Zinc finger protein 404 |
| FLJ21127 | tectonic 1 |
| GBP2 | guanylate binding protein 2, interferon-inducible |
| ST3GAL6 | ST3 beta-galactoside alpha-2,3-sialyltransferase 6 |
| PLAUR | plasminogen activator, urokinase receptor |
| NCF4 | neutrophil cytosolic factor 4, 40 kDa |
| JAK2 | Janus kinase 2 (a protein tyrosine kinase) |
| SREBF1 | sterol regulatory element binding transcription factor 1 |
| SELM | selenoprotein M |
| PPP3R1 | protein phosphatase 3 (formerly 2B), regulatory subunit B, alpha isoform |
| PRKCA | protein kinase C, alpha |
| PLA2G4C | phospholipase A2, group IVC (cytosolic, calcium-independent) |
| GBP4 | guanylate binding protein 4 |
| HIP1 | huntingtin interacting protein 1 |
| PGM2 | phosphoglucomutase 2 |
| | KIAA1632 |
| S100P | S100 calcium binding protein P |
| IL27RA | interleukin 27 receptor, alpha |
| IL15 | interleukin 15 |
| FHIT | fragile histidine triad gene |
| FAM84B | family with sequence similarity 84, member B |
| MCCC1 | methylcrotonoyl-Coenzyme A carboxylase 1 (alpha) |
| ACOT7 | acyl-CoA thioesterase 7 |
| TNFRSF12A | tumor necrosis factor receptor superfamily, member 12A |
| SF3A1 | splicing factor 3a, subunit 1, 120 kDa |
| TNFSF14 | tumor necrosis factor (ligand) superfamily, member 14 |
| CD300LB | CD300 molecule-like family member b |
| ANPEP | alanyl (membrane) aminopeptidase (aminopeptidase N, aminopeptidase M, microsomal |
| | aminopeptidase, CD13, p150) |
| FAM7A1 | |
| RHD | Rh blood group, D antigen |
| HOM-TES- | hypothetical protein LOC25900 |
| 103 | |
| CCDC41 | coiled-coil domain containing 41 |
| CRNKL1 | crooked neck pre-mRNA splicing factor-like 1 (Drosophila) |
| NCF1 | neutrophil cytosolic factor 1, (chronic granulomatous disease, autosomal 1) |
| UBTD1 | ubiquitin domain containing 1 |
| FLJ22471 | coiled-coil domain containing 92 |
| FAM101B | family with sequence similarity 101, member B |
| LOC284757 | |
| LOC649377 | |
| CDK5R1 | cyclin-dependent kinase 5, regulatory subunit 1 (p35) |
| | Full-length cDNA clone CS0DC025YP03 of Neuroblastoma Cot 25-normalized of Homo |
| | sapiens (human) |
| MBNL3 | muscleblind-like 3 (Drosophila) |
| PSTPIP1 | proline-serine-threonine phosphatase interacting protein 1 |
| WARS | tryptophanyl-tRNA synthetase |
| HLA-DPB1 | major histocompatibility complex, class II, DP beta 1 |
| LOC652616 | |
| ACTA2 | actin, alpha 2, smooth muscle, aorta |
| IBRDC3 | IBR domain containing 3 |
| PHKG2 | phosphorylase kinase, gamma 2 (testis) |
| | Phosphatidylinositol-4-phosphate 5-kinase, type II, alpha |
| LOC642035 | |
| AMFR | |
| RGS19 | regulator of G-protein signalling 19 |
| C22orf5 | chromosome 22 open reading frame 5 |
| ATF3 | activating transcription factor 3 |
| SIPA1L1 | signal-induced proliferation-associated 1 like 1 |
| MRPS34 | mitochondrial ribosomal protein S34 |
| ADAL | adenosine deaminase-like |
| NDUFAF1 | NADH dehydrogenase (ubiquinone) 1 alpha subcomplex, assembly factor 1 |
| CRAT | carnitine acetyltransferase |
| STX11 | syntaxin 11 |

Different and reciprocal immune signatures in active and latent TB are revealed using a modular approach. To yield further information on pathogenesis, the normalised per chip data was then further analyzed using a recently described stable modular analysis framework based on pre-defined clusters of genes transcripts shown to be coordinately expressed across a wide range of diseases, and often representing a cluster of molecules or cells related at a function level (Chaussabel et al., 2008, Immunity).

As the aim of this analysis was to yield functional information about genes contained within the transcriptional signatures for each group, the analysis was focused on subsets of patients found to cluster tightly together in our previous analyses, excluding outliers, reasoning that such groups would be more likely to reveal common pathways and processes involved in the disease process.

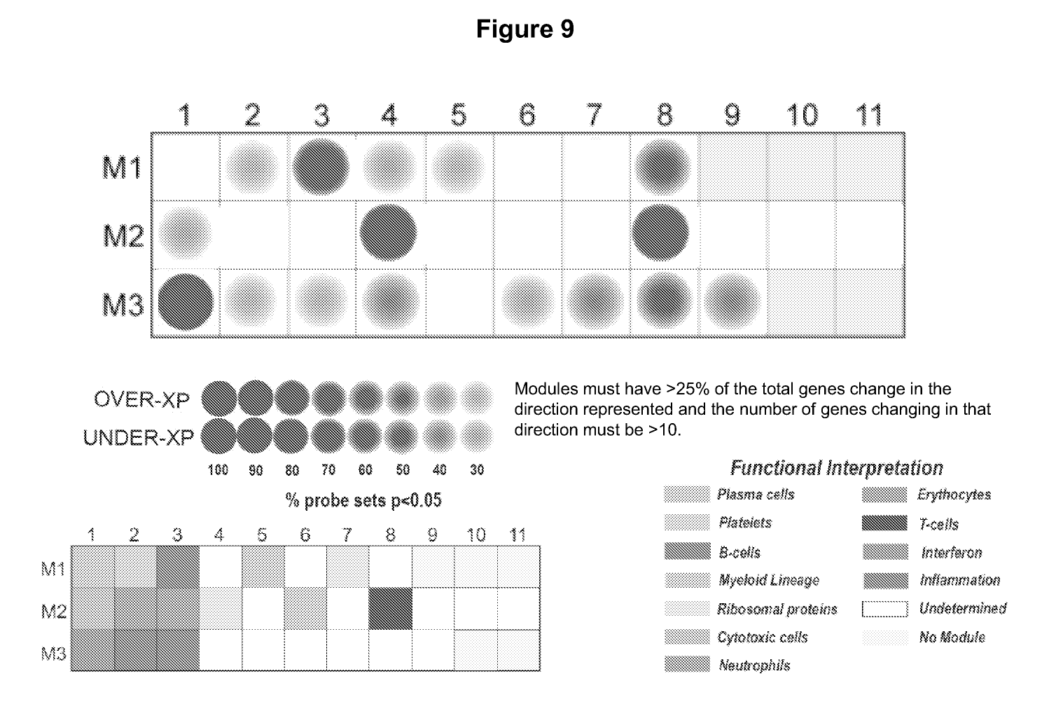

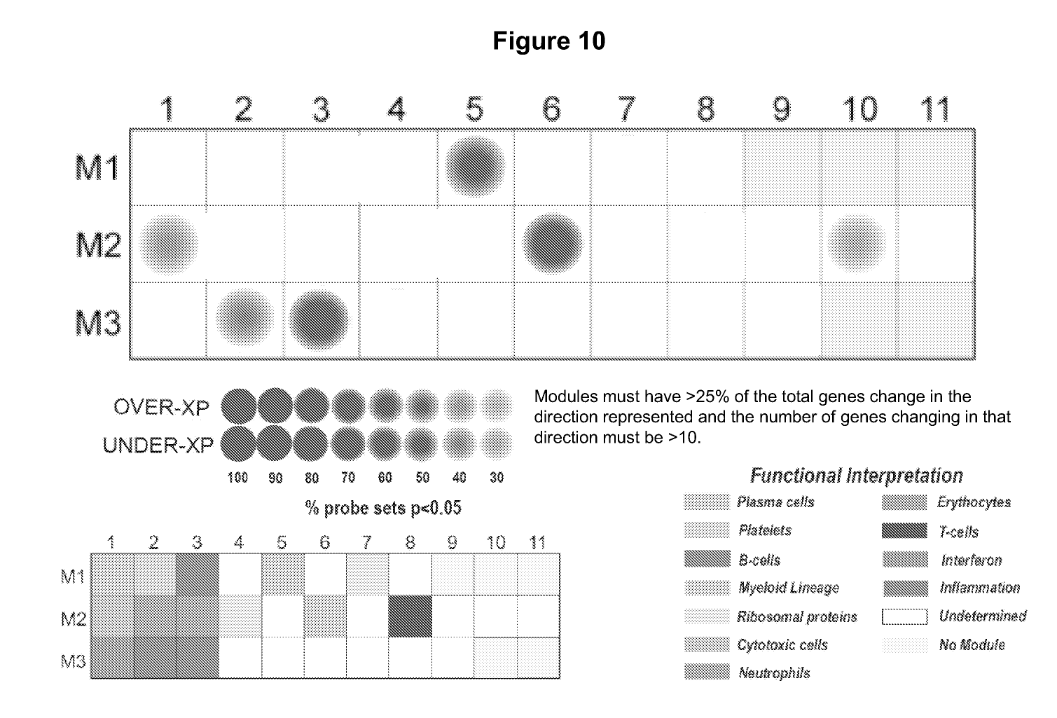

Nine patients with active TB, six healthy controls and nine patients with latent TB were selected and used in the modular analysis. Each comparison was performed separately, thus nine active TB patients were compared with six healthy controls in one analysis, and then nine latent TB patients were compared with the same six healthy controls in a separate analysis. Transcripts were filtered to exclude any not detected in at least two individuals from either group being compared. Statistical comparisons between patient and healthy control groups were then performed (Non parametric Wilcoxon-Mann-Whitney test, P<0.05), in order to identify genes that were differentially expressed between the patient group and healthy controls. These differentially expressed genes were then separated into those upregulated/overrepresented in disease group compared with control, and those down-regulated/underrepresented in disease group compared with control. These lists are then analysed on a module by module basis. Differentially expressed genes are either predominantly over-expressed or predominantly under-expressed in each module. To ensure validity each module must have >25% of the total genes change in the direction represented and the number of genes changing in a particular direction must be >10. To graphically present the global transcriptional changes, in active TB versus healthy control, or latent TB versus healthy controls, spots are aligned on a grid, with each position corresponding to a different module based on their original definition Spot intensity indicates proportion of differentially expressed transcripts changing in the direction shown out of the total number of transcripts detected for that module, while spot color indicates the polarity of the change (red: overexpressed/represented, blue: underexpressed/represented). In addition, modules' coordinates can be associated to functional annotations to facilitate data interpretation (Chaussabel, Immunity, 2008; and FIGS. 9 and 10).

A modular map of active TB compared to healthy control (FIG. 9, Table 7A-P; and Table 8) was shown to be distinct to the map of latent TB as compared to healthy controls (FIG. 10, Table 7A-F; and Table 9). In fact these independently derived module maps from active TB and latent TB show an inverse pattern of gene expression/representation, in modules which show changes in both disease states when compared with healthy controls. Genes in module M2.1 associated with cytotoxic cells were underexpressed/represented (36% -18 genes underexpressed/represented out of 50 detected in the module, genes listed in Table 6F) in active TB and yet overexpressed/represented (43% -22 genes overexpressed/represented out of 51 detected in the module, genes listed in Table 7B) in latent TB. On the other hand, a number of genes in M3.2 and M3.3 (“inflammation”) (genes listed in Tables 6J and 6K) were overexpressed/represented in active TB patients but underexpressed/represented in latent TB patients (genes listed in Table 7E and 7F). Likewise genes in M1.5 (“myeloid lineage”) were overexpressed/represented in active TB (genes listed in Table 6D) whereas they were underexpressed/represented in latent TB (genes listed in Table 7A). Genes in a module M2.10, which did not form a coherent functional module but consisted of an apparently diverse set of genes, were underexpressed/represented in latent TB (genes listed in Table 7D) but not over or underexpressed/represented in active TB as compared to controls. One of these genes is the toll-like receptor adaptor, TRAM, which is downstream of TLR-4 (LPS) and TLR-3 (dsRNA) signalling (Akira, Nat. Rev. Imm.).

For Tables 7A to 7O, relative normalized expression for active TB is given as expression in active patients relative to control. In Tables 8A to 8F, relative normalized expression for latent TB is given as expression in healthy controls relative to latent patients.

TABLE 7A
M1.2 PTB v. Control, Genes Overrepresented in Active TB.

| Relative | | | |
|---|---|---|---|
| normalised | | | |
| expression | Common Name | Gene Symbol | Description |
| | P22_15_PTBvCSelect_09May08_PAL2Ttest_UP_M1.2 | | |
| 2.447 | KX; X1k; XKR1 | XK | X-linked Kx blood group (McLeod |
| | | | syndrome) |
| 2.239 | CD62; GRMP; PSEL; CD62P; | SELP | selectin P (granule membrane protein |
| | GMP140; PADGEM; FLJ45155 | | 140 kDa, antigen CD62) |
| 2.161 | URG | EGF | epidermal growth factor (beta-urogastrone) |
| 2.133 | JAMC; JAM-C; FLJ14529 | JAM3 | junctional adhesion molecule 3 |
| 2.13 | H2B; GL105; H2B.1; H2B/q; | HIST2H2BE | histone cluster 2, H2be |
| | H2BFQ; MGC129733; | | |
| | MGC129734 | | |
| 1.889 | 4.1O; P410; EPB41L4O; | FRMD3 | FERM domain containing 3 |
| | MGC20553; RP11-439K3.2 | | |
| 1.875 | CKLFSF5; FLJ37521 | CMTM5 | CKLF-like MARVEL transmembrane domain |
| | | | containing 5 |
| 1.829 | ECM; MMRN; GPIa*; EMILIN4 | MMRN1 | multimerin 1 |
| 1.757 | PSA; PROS; PS21; PS22; PS23; | PROS1 | protein S (alpha) |
| | PS24; PS25; PS 26; Protein S; | | |
| | protein Sa | | |
| 1.752 | F13A | F13A1 | coagulation factor XIII, A1 polypeptide |
| 1.698 | H2B/S; H2BFT; H2BFAiii; | HIST1H2BK | histone cluster 1, H2bk |
| | MGC131989 | | |
| 1.638 | | RTN2 | |
| 1.59 | TMSA; HTM-alpha; TPM1-alpha; | TPM1 | tropomyosin 1 (alpha) |
| | TPM1-kappa | | |
| 1.419 | | C6orf79 | |
| 1.408 | BSS; GP1B; CD42B; MGC34595; | GP1BA | glycoprotein Ib (platelet), alpha polypeptide |
| | CD42b-alpha | | |
| 1.338 | CD61; GP3A; GPIIIa | ITGB3 | integrin, beta 3 (platelet glycoprotein IIIa, |
| | | | antigen CD61) |
| 1.183 | CMIP; KIAA1694 | CMIP | c-Maf-inducing protein |

TABLE 7B
M1.3 PTB v. Control, Genes Underrepresented in Active TB.

| Relative | | | |
|---|---|---|---|
| normalised | | Gene | |
| expression | Common Name | Symbol | Description |
| | P22_15_PTBvCSelect_09May08_PAL2Ttest_DOWN_M1.3 | | |
| 0.82 | FLJ31738; KIAA1209 | PLEKHG1 | pleckstrin homology domain containing, |
| | | | family G (with RhoGef domain) member 1 |
| 0.778 | SPI-B | SPIB | Spi-B transcription factor (Spi-1/PU.1 |
| | | | related) |
| 0.767 | EVI9; CTIP1; BCL11A-L; | BCL11A | B-cell CLL/lymphoma 11A (zinc finger |
| | BCL11A-S; FLJ10173; FLJ34997; | | protein) |
| | KIAA1809; BCL11A-XL | | |
| 0.715 | MGC20446 | CYBASC3 | cytochrome b, ascorbate dependent 3 |
| 0.677 | NIDD; MGC42530 | ZDHHC23 | zinc finger, DHHC-type containing 23 |
| 0.629 | ESG; ESG1; GRG1 | TLE1 | transducin-like enhancer of split 1 (E(sp1) |
| | | | homolog, Drosophila) |
| 0.612 | B29; IGB | CD79B | CD79b molecule, immunoglobulin- |
| | | | associated beta |
| 0.581 | LYB2; CD72b | CD72 | CD72 molecule |
| 0.559 | KIAA0977 | COBLL1 | COBL-like 1 |
| 0.556 | BASH; Ly57; SLP65; BLNK-s; | BLNK | B-cell linker |
| | SLP-65; MGC111051 | | |
| 0.543 | TCL1 | TCL1A | T-cell leukemia/lymphoma 1A |
| 0.518 | c-Myc | MYC | v-myc myelocytomatosis viral oncogene |
| | | | homolog (avian) |
| 0.512 | BANK; FLJ20706; FLJ34204 | BANK1 | B-cell scaffold protein with ankyrin repeats 1 |
| 0.51 | B4; MGC12802 | CD19 | CD19 molecule |
| 0.496 | FCRH1; IFGP1; IRTA5; RP11- | FCRL1 | Fc receptor-like 1 |
| | 367J7.7; DKFZp667O1421 | | |
| 0.487 | FLJ00058 | GNG7 | guanine nucleotide binding protein (G |
| | | | protein), gamma 7 |
| 0.482 | FLJ21562; FLJ43762 | C13orf18 | chromosome 13 open reading frame 18 |
| 0.477 | BRDG1; STAP1 | BRDG1 | BCR downstream signaling 1 |
| 0.471 | MGC10442 | BLK | B lymphoid tyrosine kinase |
| 0.467 | R1; JPO2; RAM2; | CDCA7L | cell division cycle associated 7-like |
| | DKFZp762L0311 | | |
| 0.445 | ORP10; OSBP9; FLJ20363 | OSBPL10 | oxysterol binding protein-like 10 |
| 0.397 | 8HS20; N27C7-2 | VPREB3 | pre-B lymphocyte gene 3 |
| 0.361 | LAF4; MLLT2-like | AFF3 | AF4/FMR2 family, member 3 |
| 0.334 | FCRL; FREB; FCRLX; FCRLb; | FCRLM1 | Fc receptor-like A |
| | FCRLd; FCRLe; FCRLM1; | | |
| | FCRLc1; FCRLc2; MGC4595; | | |
| | RP11-474I16.5 | | |

TABLE 7C
M1.4 PTB v. Control, Genes Underrepresented in Active TB.

| Relative | | | |
|---|---|---|---|
| normalised | | Gene | |
| expression | Common Name | Symbol | Description |
| | P22_15_PTBvCSelect_09May08_PAL2Ttest_DOWN_M1.4 | | |
| 0.907 | FLJ12298; ZKSCAN14 | ZNF394 | zinc finger protein 394 |
| 0.835 | JMY; FLJ37870; MGC163496 | JMY | junction-mediating and regulatory protein |
| 0.825 | C1; C2; HNRNP; SNRPC; | HNRPC | heterogeneous nuclear ribonucleoprotein C |
| | hnRNPC; MGC104306; | | (C1/C2) |
| | MGC105117; MGC117353; | | |
| | MGC131677 | | |
| 0.78 | SON3; BASS1; DBP-5; | SON | SON DNA binding protein |
| | NREBP; C21orf50; FLJ21099; | | |
| | FLJ33914; KIAA1019 | | |
| 0.77 | HMGE; FLJ25609 | GRPEL1 | GrpE-like 1, mitochondrial (E. coli) |
| 0.747 | HEPP; FLJ20764; MGC19517 | CDCA4 | cell division cycle associated 4 |
| 0.723 | RITA; ZNF361; ZNF463; | ZNF331 | zinc finger protein 331 |
| | DKFZp686L0787 | | |
| 0.698 | FLJ12670; FLJ20436 | C12orf41 | chromosome 12 open reading frame 41 |
| 0.698 | DRBF; MMP4; MPP4; NF90; | ILF3 | interleukin enhancer binding factor 3, |
| | NFAR; TCP80; DRBP76; | | 90 kDa |
| | NFAR-1; MPHOSPH4; NF- | | |
| | AT-90 | | |
| 0.689 | TIMAP; ANKRD4; KIAA0823 | PPP1R16B | protein phosphatase 1, regulatory (inhibitor) |
| | | | subunit 16B |
| 0.678 | PRP21; PRPF21; SAP114; | SF3A1 | splicing factor 3a, subunit 1, 120 kDa |
| | SF3A120 | | |
| 0.667 | SDS; SWDS; CGI-97; | SBDS | Shwachman-Bodian-Diamond syndrome |
| | FLJ10917 | | |
| 0.665 | BL11; HB15 | CD83 | CD83 molecule |
| 0.645 | NOT; RNR1; HZF-3; NURR1; | NR4A2 | nuclear receptor subfamily 4, group A, |
| | TINUR | | member 2 |
| 0.62 | H1RNA | RNASEH1 | ribonuclease H1 |

TABLE 7D
M1.5 PTB v. Control, Genes Overrepresented in Active TB.

| Relative | | | |
|---|---|---|---|
| normalised | | | |
| expression | Common Name | Gene Symbol | Description |
| | P22_15_PTBvCSelect_09May08_PAL2Ttest_UP_M1.5 | | |
| 2.384 | VHR | DUSP3 | dual specificity phosphatase 3 (vaccinia |
| | | | virus phosphatase VH1-related) |
| 2.139 | 4.1B; DAL1; DAL-1; | EPB41L3 | erythrocyte membrane protein band 4.1-like 3 |
| | FLJ37633; KIAA0987 | | |
| 2.014 | HXK3; HKIII | HK3 | hexokinase 3 (white cell) |
| 1.972 | HL14; MGC75071 | LGALS2 | lectin, galactoside-binding, soluble, 2 |
| 1.844 | KYNU | KYNU | kynureninase (L-kynurenine hydrolase) |
| 1.618 | BLVR; BVRA | BLVRA | biliverdin reductase A |
| 1.594 | RP35; SEMB; SEMAB; | SEMA4A | sema domain, immunoglobulin domain (Ig), |
| | CORD10; FLJ12287; RP11- | | transmembrane domain (TM) and short |
| | 54H19.2 | | cytoplasmic domain, (semaphorin) 4A |
| 1.535 | | GRN | |
| 1.531 | G6S; MGC21274 | GNS | glucosamine (N-acetyl)-6-sulfatase |
| | | | (Sanfilippo disease IIID) |
| 1.524 | FOAP-10; EMILIN-2; | EMILIN2 | elastin microfibril interfacer 2 |
| | FLJ33200 | | |
| 1.507 | cent-b; HSA272195 | CENTA2 | centaurin, alpha 2 |
| 1.449 | APPS; CPSB | CTSB | cathepsin B |
| 1.438 | ASGPR; CLEC4H1; Hs.12056 | ASGR1 | asialoglycoprotein receptor 1 |
| 1.433 | CD32; FCG2; FcGR; CD32A; | FCGR2A | Fc fragment of IgG, low affinity IIa, |
| | CDw32; FCGR2; IGFR2; | | receptor (CD32) |
| | FCGR2A1; MGC23887; | | |
| | MGC30032 | | |
| 1.425 | TIL4; CD282 | TLR2 | toll-like receptor 2 |
| 1.424 | PI; A1A; AAT; PI1; A1AT; | SERPINA1 | serpin peptidase inhibitor, clade A (alpha-1 |
| | MGC9222; PRO2275; | | antiproteinase, antitrypsin), member 1 |
| | MGC23330 | | |
| 1.413 | TEM7R; FLJ14623 | PLXDC2 | plexin domain containing 2 |
| 1.41 | CD14 | CD14 | CD14 molecule |
| 1.398 | Rab22B | RAB31 | RAB31, member RAS oncogene family |
| 1.386 | FEX1; FEEL-1; FELE-1; | STAB1 | stabilin 1 |
| | STAB-1; CLEVER-1; | | |
| | KIAA0246 | | |
| 1.352 | MYD88 | MYD88 | myeloid differentiation primary response |
| | | | gene (88) |
| 1.349 | MLN70; S100C | S100A11 | S100 calcium binding protein A11 |
| 1.347 | FLJ22662 | FLJ22662 | hypothetical protein FLJ22662 |
| 1.346 | CLN2; GIG1; LPIC; TPP I; | TPP1 | tripeptidyl peptidase I |
| | MGC21297 | | |
| 1.251 | p75; TBPII; TNFBR; TNFR2; | TNFRSF1B | tumor necrosis factor receptor superfamily, |
| | CD120b; TNFR80; TNF-R75; | | member 1B |
| | p75TNFR; TNF-R-II | | |
| 1.239 | JTK9 | HCK | hemopoietic cell kinase |
| 1.172 | IBA1; AIF-1; IRT-1 | AIF1 | allograft inflammatory factor 1 |

TABLE 7E
M1.8 PTB v. Control, Genes Underrepresented in Active TB.

| Relative | | | |
|---|---|---|---|
| normalised | | Gene | |
| expression | Common Name | Symbol | Description |
| | P22_15_PTBvCSelect_09May08_PAL2Ttest_DOWN_M1.8 | | |
| 0.878 | DBP2; PRP8; DDX16; | DHX16 | DEAH (Asp-Glu-Ala-His) box polypeptide |
| | PRO2014 | | 16 |
| 0.858 | AN11; HAN11 | WDR68 | WD repeat domain 68 |
| 0.843 | NDR; NDR1 | STK38 | serine/threonine kinase 38 |
| 0.821 | FLJ20097; FLJ23581; | FLJ20097 | coiled-coil domain containing 132 |
| | KIAA1861 | | |
| 0.814 | FLJ42526; FLJ45813; | RSBN1L | round spermatid basic protein 1-like |
| | MGC71764 | | |
| 0.809 | C9orf55; C9orf55B; FLJ20686; | DENND4C | DENN/MADD domain containing 4C |
| | bA513M16.3; | | |
| | DKFZp686I09113 | | |
| 0.808 | SON3; BASS1; DBP-5; | SON | SON DNA binding protein |
| | NREBP; C21orf50; FLJ21099; | | |
| | FLJ33914; KIAA1019 | | |
| 0.807 | p150; VPS15; MGC102700 | PIK3R4 | phosphoinositide-3-kinase, regulatory |
| | | | subunit 4, p150 |
| 0.8 | 4E-T; Clast4; FLJ21601; | EIF4ENIF1 | eukaryotic translation initiation factor 4E |
| | FLJ26551 | | nuclear import factor 1 |
| 0.798 | TAF2D; TAFII100 | TAF5 | TAF5 RNA polymerase II, TATA box |
| | | | binding protein (TBP)-associated factor, |
| | | | 100 kDa |
| 0.793 | DBR1 | DBR1 | debranching enzyme homolog 1 |
| | | | (S. cerevisiae) |
| 0.785 | SMAP; p120; SMAP2 | BRD8 | bromodomain containing 8 |
| 0.785 | | CASP2 | |
| 0.772 | TRF2; TRBF2 | TERF2 | telomeric repeat binding factor 2 |
| 0.772 | hNUP133; FLJ10814; | NUP133 | nucleoporin 133 kDa |
| | MGC21133 | | |
| 0.762 | MGC4268; FLJ38552 | MGC4268 | AMME chromosomal region gene 1-like |
| 0.761 | PUMH2; PUML2; FLJ36528; | PUM2 | pumilio homolog 2 (Drosophila) |
| | KIAA0235; MGC138251; | | |
| | MGC138253 | | |
| 0.751 | BYE1; DIO1; DATF1; DIDO2; | DIDO1 | death inducer-obliterator 1 |
| | DIDO3; DIO-1; FLJ11265; | | |
| | KIAA0333; MGC16140; | | |
| | C20orf158; dJ885L7.8; | | |
| | DKFZp434P1115 | | |
| 0.738 | KOX5; ZNF13 | ZNF45 | zinc finger protein 45 |
| 0.727 | FLJ20558 | FLJ20558 | chromosome 2 open reading frame 42 |
| 0.713 | FLJ32343 | CWF19L2 | CWF19-like 2, cell cycle control (S. pombe) |
| 0.709 | MGC16770 | RAB22A | RAB22A, member RAS oncogene family |
| 0.708 | FLJ14431 | CBR4 | carbonyl reductase 4 |
| 0.704 | AASDH; NRPS998; | AASDH | 2-aminoadipic 6-semialdehyde |
| | NRPS1098 | | dehydrogenase |
| 0.698 | ZSCAN11 | ZNF232 | zinc finger protein 232 |
| 0.692 | NudCL; KIAA1068 | NUDCD3 | NudC domain containing 3 |
| 0.691 | CCA1; MtCCA; CGI-47 | TRNT1 | tRNA nucleotidyl transferase, |
| | | | CCA-adding, 1 |
| 0.689 | RBM30; RBM4L; ZCRB3B; | RBM4B | RNA binding motif protein 4B |
| | ZCCHC15; MGC10871 | | |
| 0.683 | CLF; CRN; HCRN; SYF3; | CRNKL1 | crooked neck pre-mRNA splicing factor- |
| | MSTP021 | | like 1 (Drosophila) |
| 0.676 | ZBU1; HLTF1; RNF80; | SMARCA3 | helicase-like transcription factor |
| | HIP116; SNF2L3; HIP116A; | | |
| | SMARCA3 | | |
| 0.666 | SWAN; KIAA0765; | RBM12 | RNA binding motif protein 12 |
| | HRIHFB2091 | | |
| 0.658 | FLJ10287; FLJ11219 | CCDC76 | coiled-coil domain containing 76 |
| 0.654 | INT5; KIAA1698 | KIAA1698 | integrator complex subunit 5 |
| 0.652 | IAN7; hIAN7; MGC27027 | GIMAP7 | GTPase, IMAP family member 7 |
| 0.651 | TTC20; DKFZP586B0923 | KIAA1279 | KIAA1279 |
| 0.65 | RAL; MGC48949 | RALA | v-ral simian leukemia viral oncogene |
| | | | homolog A (ras related) |
| 0.639 | MPRB; LMPB1; C6orf33; | PAQR8 | progestin and adipoQ receptor family |
| | FLJ32521; FLJ46206 | | member VIII |
| 0.634 | FLJ11171 | FLJ11171 | hypothetical protein FLJ11171 |
| 0.613 | LCF; IL-16; prIL-16; | IL16 | interleukin 16 (lymphocyte chemoattractant |
| | FLJ16806; FLJ42735; | | factor) |
| | FLJ44234; HsT19289 | | |
| 0.611 | FLJ33226; 1190004M21Rik | PYGO2 | pygopus homolog 2 (Drosophila) |
| 0.577 | GLC1G; UTP21; TAWDRP; | WDR36 | WD repeat domain 36 |
| | TA-WDRP; DKFZp686I1650 | | |
| 0.574 | FLJ20287; bA208F1.2; RP11- | TEX10 | testis expressed 10 |
| | 208F1.2 | | |
| 0.568 | KIAA1982 | ZNF721 | zinc finger protein 721 |
| 0.55 | FLJ22457; RP5-1180E21.2 | DENND2D | DENN/MADD domain containing 2D |
| 0.545 | ozrf1; ZFP260 | ZFP260 | zinc finger protein 260 |
| 0.491 | GLS1; FLJ10358; KIAA0838; | GLS | glutaminase |
| | DKFZp686O15119 | | |

TABLE 7F
M2.1 PTB v. Control, Genes Underrepresented in Active TB.

| Relative | | | |
|---|---|---|---|
| normalised | | Gene | |
| expression | Common Name | Symbol | Description |
| | P22_15_PTBvCSelect_09May08_PAL2Ttest_DOWN_M2.01 | | |
| 0.712 | PTPMEG; PTPMEG1 | PTPN4 | protein tyrosine phosphatase, non-receptor |
| | | | type 4 (megakaryocyte) |
| 0.665 | FLJ34563; MGC35163 | SAMD3 | sterile alpha motif domain containing 3 |
| 0.643 | STAT4 | STAT4 | signal transducer and activator of |
| | | | transcription 4 |
| 0.638 | DIL1; DIL-1; Mindin; M- | SPON2 | spondin 2, extracellular matrix protein |
| | spondin | | |
| 0.631 | SLP2; SGA72M; CHR11SYT; | SYTL2 | synaptotagmin-like 2 |
| | KIAA1597; MGC102768 | | |
| 0.628 | DORZ1; DKFZP564O243 | ABHD14A | abhydrolase domain containing 14A |
| 0.615 | LPAP; CD45-AP; | PTPRCAP | protein tyrosine phosphatase, receptor |
| | MGC138602; MGC138603 | | type, C-associated protein |
| 0.595 | PKCL; PKC-L; PRKCL; | PRKCH | protein kinase C, eta |
| | MGC5363; MGC26269; | | |
| | nPKC-eta | | |
| 0.581 | MGC33870; MGC74858 | NCALD | neurocalcin delta |
| 0.566 | T11; SRBC | CD2 | CD2 molecule |
| 0.554 | KLR; CD314; NKG2D; NKG2- | KLRK1 | killer cell lectin-like receptor subfamily K, |
| | D; D12S2489E | | member 1 |
| 0.546 | LAX; FLJ20340 | LAX1 | lymphocyte transmembrane adaptor 1 |
| 0.529 | CD122; P70-75 | IL2RB | interleukin 2 receptor, beta |
| 0.515 | FEZ1 | FEZ1 | fasciculation and elongation protein zeta 1 |
| | | | (zygin I) |
| 0.509 | CHK; CTK; HYL; Lsk; | MATK | megakaryocyte-associated tyrosine kinase |
| | HYLTK; HHYLTK; | | |
| | MGC1708; MGC2101; | | |
| | DKFZp434N1212 | | |
| 0.468 | CLIC3 | CLIC3 | chloride intracellular channel 3 |
| 0.439 | 1C7; CD337; LY117; NKp30 | NCR3 | natural cytotoxicity triggering receptor 3 |
| 0.39 | TRYP2 | GZMK | granzyme K (granzyme 3; tryptase II) |

TABLE 7G
M2.4 PTB v. Control, Genes Underrepresented in Active TB.

| Relative | | | |
|---|---|---|---|
| normalised | | Gene | |
| expression | Common Name | Symbol | Description |
| | P22_15_PTBvCSelect_09May08_PAL2Ttest_DOWN_M2.04 | | |
| 0.858 | ATPO; OSCP | ATP5O | ATP synthase, H+ transporting, |
| | | | mitochondrial F1 complex, O subunit |
| | | | (oligomycin sensitivity conferring protein) |
| 0.831 | M9; eIF3k; ARG134; PTD001; | EIF3S12 | eukaryotic translation initiation factor 3, |
| | HSPC029; MSTP001; PLAC- | | subunit 12 |
| | 24; PRO1474 | | |
| 0.822 | RPL8 | RPL8 | ribosomal protein L8 |
| 0.811 | EF2; EEF-2 | EEF2 | eukaryotic translation elongation factor 2 |
| 0.804 | RPB9; hRPB14.5 | POLR2I | polymerase (RNA) II (DNA directed) |
| | | | polypeptide I, 14.5 kDa |
| 0.801 | RP8; ZMYND7; MGC12347 | PDCD2 | programmed cell death 2 |
| 0.788 | ARI2; TRIAD1; FLJ10938; | ARIH2 | ariadne homolog 2 (Drosophila) |
| | FLJ33921 | | |
| 0.776 | Erv46; CGI-54; PRO0989; | ERGIC3 | ERGIC and golgi 3 |
| | C20orf47; NY-BR-84; | | |
| | SDBCAG84; dJ477O4.2 | | |
| 0.771 | ART-27 | UXT | ubiquitously-expressed transcript |
| 0.769 | H12.3; HLC-7; PIG21; | GNB2L1 | guanine nucleotide binding protein (G |
| | RACK1; Gnb2-rs1 | | protein), beta polypeptide 2-like 1 |
| 0.766 | eIF3h; eIF3-p40; MGC102958; | EIF3S3 | eukaryotic translation initiation factor 3, |
| | eIF3-gamma | | subunit 3 gamma, 40 kDa |
| 0.759 | HCA56 | LGTN | ligatin |
| 0.758 | 2PP2A; IGAAD; I2PP2A; | SET | SET translocation (myeloid leukemia- |
| | PHAPII; TAF-IBETA | | associated) |
| 0.752 | ANG2 | C11orf2 | chromosome 11 open reading frame2 |
| 0.74 | C6.1B | MTCP1 | mature T-cell proliferation 1 |
| 0.736 | LCP; HCLP-1 | KLHDC2 | kelch domain containing 2 |
| 0.722 | DKFZP566B023 | RPL36 | ribosomal protein L36 |
| 0.712 | KOX30 | ZNF32 | zinc finger protein 32 |
| 0.71 | AMP; MGC125856; | APRT | adenine phosphoribosyltransferase |
| | MGC125857; MGC129961; | | |
| | DKFZp686D13177 | | |
| 0.694 | GDH; MGC149525; | CRYL1 | crystallin, lambda 1 |
| | MGC149526; lambda-CRY | | |
| 0.689 | FLJ27451; MGC102930 | RPS20 | ribosomal protein S20 |
| 0.686 | INT6; eIF3e; EIF3-P48; eIF3- | EIF3S6 | eukaryotic translation initiation factor 3, |
| | p46 | | subunit 6 48 kDa |
| 0.68 | LK4; hCERK; FLJ21430; | CERK | ceramide kinase |
| | FLJ23239; KIAA1646; | | |
| | MGC131878; dA59H18.2; | | |
| | dA59H18.3; DKFZp434E0211 | | |
| 0.675 | HINT; PKCI-1; PRKCNH1 | HINT1 | histidine triad nucleotide binding protein 1 |
| 0.675 | NHP2; NHP2P | NOLA2 | nucleolar protein family A, member 2 |
| | | | (H/ACA small nucleolar RNPs) |
| 0.668 | AMP; MGC125856; | APRT | adenine phosphoribosyltransferase |
| | MGC125857; MGC129961; | | |
| | DKFZp686D13177 | | |
| 0.667 | TOM7 | TOMM7 | translocase of outer mitochondrial |
| | | | membrane 7 homolog (yeast) |
| 0.655 | SIVA; CD27BP; Siva-1; Siva-2 | SIVA | SIVA1, apoptosis-inducing factor |
| 0.646 | PBP; HCNP; PEBP; RKIP | PEBP1 | phosphatidylethanolamine binding |
| | | | protein 1 |
| 0.628 | PRP9; PRPF9; SAP61; SF3a60 | SF3A3 | splicing factor 3a, subunit 3, 60 kDa |
| 0.62 | FLJ12525; dJ475B7.2; RP3- | LAS1L | LAS1-like (S. cerevisiae) |
| | 475B7.2 | | |
| 0.593 | EC45; RPL10; RPLY10; | RPL15 | ribosomal protein L15 |
| | RPYL10; FLJ26304; | | |
| | MGC88603 | | |
| 0.567 | HNRNP; JKTBP; JKTBP2; | HNRPDL | heterogeneous nuclear ribonucleoprotein |
| | laAUF1 | | D-like |
| 0.562 | SMD2; SNRPD1 | SNRPD2 | small nuclear ribonucleoprotein D2 |
| | | | polypeptide 16.5 kDa |
| 0.549 | | PPIA | |
| 0.527 | LOC130074; MGC87527 | LOC130074 | p20 |
| 0.524 | RDGBB; RDGBB1; RDGB- | PITPNC1 | phosphatidylinositol transfer protein, |
| | BETA | | cytoplasmic 1 |
| 0.5 | HEI10; C14orf18 | CCNB1IP1 | cyclin B1 interacting protein 1 |
| 0.492 | EAP; HBP15; HBP15/L22 | RPL22 | ribosomal protein L22 |

TABLE 7H
M2.8 PTB v. Control, Genes Underrepresented in Active TB.

| Relative | | | |
|---|---|---|---|
| normalised | | | |
| expression | Common Name | Gene Symbol | Description |
| | P22_15_PTBvCSelect_09May08_PAL2Ttest_DOWN_M2.08 | | |
| 0.871 | KPL1; PHR1; PHRET1 | PLEKHB1 | pleckstrin homology domain containing, |
| | | | family B (evectins) member 1 |
| 0.816 | MGC132014 | INPP4B | inositol polyphosphate-4-phosphatase, type |
| | | | II, 105 kDa |
| 0.732 | SEP2; SEPT2; KIAA0128; | 6-Sep | septin 6 |
| | MGC16619; MGC20339; RP5- | | |
| | 876A24.2 | | |
| 0.711 | GIL | AQP3 | aquaporin 3 (Gill blood group) |
| 0.691 | FLJ36386 | LZTFL1 | leucine zipper transcription factor-like 1 |
| 0.67 | p52; p75; PAIP; DFS70; | PSIP1 | PC4 and SFRS1 interacting protein 1 |
| | LEDGF; PSIP2; MGC74712 | | |
| 0.669 | GRG; ESP1; GRG5; TLE5; | AES | amino-terminal enhancer of split |
| | AES-1; AES-2 | | |
| 0.668 | p33; TNFC; TNFSF3 | LTB | lymphotoxin beta (TNF superfamily, |
| | | | member 3) |
| 0.646 | KIAA0521; MGC15913 | ARHGEF18 | rho/rac guanine nucleotide exchange factor |
| | | | (GEF) 18 |
| 0.634 | TEM3; TEM7; FLJ36270; | PLXDC1 | plexin domain containing 1 |
| | FLJ45632; DKFZp686F0937 | | |
| 0.626 | HPIP | PBXIP1 | pre-B-cell leukemia homeobox interacting |
| | | | protein 1 |
| 0.621 | KIAA0495; MGC138189 | KIAA0495 | KIAA0495 |
| 0.615 | KUP; ZNF46 | ZBTB25 | zinc finger and BTB domain containing 25 |
| 0.61 | FLJ20729; FLJ20760; NY-BR- | C1orf181 | chromosome 1 open reading frame 181 |
| | 75; MGC131963 | | |
| 0.609 | AAG6; PKCA; PRKACA; | PRKCA | protein kinase C, alpha |
| | MGC129900; MGC129901; | | |
| | PKC-alpha | | |
| 0.604 | CGI-25 | NOSIP | nitric oxide synthase interacting protein |
| 0.602 | FLJ20152; FLJ22155; | FLJ20152 | family with sequence similarity 134, |
| | FLJ22179 | | member B |
| 0.599 | FRA3B; AP3Aase | FHIT | fragile histidine triad gene |
| 0.596 | WDR74 | WDR74 | WD repeat domain 74; synonyms: |
| | | | FLJ10439, FLJ21730; Homo sapiens WD |
| | | | repeat domain 74 (WDR74), mRNA. |
| 0.595 | E25A; BRICD2A | ITM2A | integral membrane protein 2A |
| 0.587 | HPF2 | ZNF84 | zinc finger protein 84 |
| 0.58 | SEK; HEK8; TYRO1 | EPHA4 | EPH receptor A4 |
| 0.578 | SID1; SID-1; FLJ20174; | SIDT1 | SID1 transmembrane family, member 1 |
| | B830021E24Rik | | |
| 0.557 | LTBP2; LTBP-3; pp6425; | LTBP3 | latent transforming growth factor beta |
| | FLJ33431; FLJ39893; | | binding protein 3 |
| | FLJ42533; FLJ44138; | | |
| | DKFZP586M2123 | | |
| 0.556 | V; RASGRP; hRasGRP1; | RASGRP1 | RAS guanyl releasing protein 1 (calcium |
| | MGC129998; MGC129999; | | and DAG-regulated) |
| | CALDAG-GEFI; CALDAG- | | |
| | GEFII | | |
| 0.546 | TTF; ARHH | RHOH | ras homolog gene family, member H |
| 0.545 | LAT3; LAT-2; y+LAT-2; | SLC7A6 | solute carrier family 7 (cationic amino acid |
| | KIAA0245; DKFZp686K15246 | | transporter, y+ system), member 6 |
| 0.541 | TP120 | CD6 | CD6 molecule |
| 0.537 | MGC29816 | CHMP7 | CHMP family, member 7 |
| 0.53 | DAGK; DAGK1; MGC12821; | DGKA | diacylglycerol kinase, alpha 80 kDa |
| | MGC42356; DGK-alpha | | |
| 0.523 | hly9; mLY9; CD229; SLAMF3 | LY9 | lymphocyte antigen 9 |
| 0.52 | EMT; LYK; PSCTK2; | ITK | IL2-inducible T-cell kinase |
| | MGC126257; MGC126258 | | |
| 0.519 | TACTILE; MGC22596; | CD96 | CD96 molecule |
| | DKFZp667E2122 | | |
| 0.518 | SEP2; SEPT2; KIAA0128; | 6-Sep | septin 6 |
| | MGC16619; MGC20339; RP5- | | |
| | 876A24.2 | | |
| 0.501 | SCAP1; SKAP55 | SCAP1 | src kinase associated phosphoprotein 1 |
| 0.49 | FLJ12884; MGC130014; | C10orf38 | chromosome 10 open reading frame 38 |
| | MGC130015 | | |
| 0.488 | T1; LEU1 | CD5 | CD5 molecule |
| 0.487 | MAL | MAL | mal, T-cell differentiation protein |
| 0.484 | SATB1 | SATB1 | SATB homeobox 1 |
| 0.48 | LDH-H; TRG-5 | LDHB | lactate dehydrogenase B |
| 0.473 | Ray; FLJ39121; | SH3YL1 | SH3 domain containing, Ysc84-like 1 (S. cerevisiae) |
| | DKFZP586F1318 | | |
| 0.466 | P19; SGRF; IL-23; IL-23A; | IL23A | interleukin 23, alpha subunit p19 |
| | IL23P19; MGC79388 | | |
| 0.465 | KE6; FABG; HKE6; FABGL; | HSD17B8 | hydroxysteroid (17-beta) dehydrogenase 8 |
| | RING2; H2-KE6; D6S2245E; | | |
| | dJ1033B10.9 | | |
| 0.456 | ARH; ARH1; ARH2; FHCB1; | LDLRAP1 | low density lipoprotein receptor adaptor |
| | FHCB2; MGC34705; | | protein 1 |
| | DKFZp586D0624 | | |
| 0.453 | MGC45416; | OCIAD2 | OCIA domain containing 2 |
| | DKFZp686C03164 | | |
| 0.451 | CD172g; SIRPB2; SIRP-B2; | SIRPB2 | signal-regulatory protein gamma |
| | bA77C3.1; SIRPgamma | | |
| 0.435 | GP40; TP41; Tp40; LEU-9 | CD7 | CD7 molecule |
| 0.427 | MGC15763 | MGC15763 | oxidoreductase NAD-binding domain |
| | | | containing 1 |
| 0.41 | AS160; DKFZp779C0666 | TBC1D4 | TBC1 domain family, member 4 |
| 0.404 | HMIC; MAN1C; MAN1A3; | MAN1C1 | mannosidase, alpha, class 1C, member 1 |
| | pp6318 | | |
| 0.401 | Tp44; MGC138290 | CD28 | CD28 molecule |
| 0.394 | FLJ12586 | ZNF329 | zinc finger protein 329 |
| 0.39 | TCF-1; MGC47735 | TCF7 | transcription factor 7 (T-cell specific, HMG- |
| | | | box) |
| 0.385 | ABLIM; LIMAB1; LIMATIN; | ABLIM1 | actin binding LIM protein 1 |
| | MGC1224; FLJ14564; | | |
| | KIAA0059; DKFZp781D0148 | | |
| 0.383 | NSE2; BCMP101 | FAM84B | family with sequence similarity 84, member B |
| 0.377 | TOSO | FAIM3 | Fas apoptotic inhibitory molecule 3 |
| 0.371 | EEIG1; C9orf132; MGC50853; | C9orf132 | family with sequence similarity 102, |
| | bA203J24.7 | | member A |
| 0.36 | RIT1; CTIP2; CTIP-2; hRIT1- | BCL11B | B-cell CLL/lymphoma 11B (zinc finger |
| | alpha | | protein) |
| 0.33 | CLP24; FLJ20898; | C16orf30 | chromosome 16 open reading frame 30 |
| | MGC111564 | | |
| 0.315 | TCF1ALPHA; | LEF1 | lymphoid enhancer-binding factor 1 |
| | DKFZp586H0919 | | |
| 0.29 | BLR2; EBI1; CD197; | CCR7 | chemokine (C-C motif) receptor 7 |
| | CDw197; CMKBR7 | | |
| 0.244 | STK37; PASKIN; KIAA0135; | PASK | PAS domain containing serine/threonine |
| | DKFZP434O051; | | kinase |
| | DKFZp686P2031 | | |
| 0.205 | NRP2 | NELL2 | NEL-like 2 (chicken) |

TABLE 7I
M3.1 PTB v. Control, Genes Overrepresented in Active TB.

| Relative | | | |
|---|---|---|---|
| normalised | | | |
| expression | Common Name | Gene Symbol | Description |
| | P22_15_PTBvCSelect_09May08_PAL2Ttest_UP_M3.1 | | |
| 17.93 | MGC22805 | ANKRD22 | ankyrin repeat domain 22 |
| 14.86 | C1IN; C1NH; HAE1; HAE2; | SERPING1 | serpin peptidase inhibitor, clade G (C1 |
| | C1INH | | inhibitor), member 1, (angioedema, |
| | | | hereditary) |
| 9.425 | cig5; vig1; 2510004L01Rik | RSAD2 | radical S-adenosyl methionine domain |
| | | | containing 2 |
| 8.938 | BRESI1; MGC29634 | EPSTI1 | epithelial stromal interaction 1 (breast) |
| 8.226 | GS3686; C1orf29 | IFI44L | interferon-induced protein 44-like |
| 7.566 | GBP1 | GBP1 | guanylate binding protein 1, interferon- |
| | | | inducible, 67 kDa |
| 5.677 | p44; MTAP44 | IFI44 | interferon-induced protein 44 |
| 4.701 | LAP; PEPS; LAPEP | LAP3 | leucine aminopeptidase 3 |
| 4.401 | IRG2; IFI60; IFIT4; ISG60; | IFIT3 | interferon-induced protein with |
| | RIG-G; CIG-49; GARG-49 | | tetratricopeptide repeats 3 |
| 4.091 | OIAS; IFI-4; OIASI | OAS1 | 2′,5′-oligoadenylate synthetase 1, 40/46 kDa |
| 3.947 | p100; MGC133260 | OAS3 | 2′-5′-oligoadenylate synthetase 3, 100 kDa |
| 3.944 | G1P2; UCRP; IFI15 | G1P2 | ISG15 ubiquitin-like modifier |
| 3.915 | UEF1; DRIF2; C7orf6; | SAMD9L | sterile alpha motif domain containing 9-like |
| | FLJ39885; KIAA2005 | | |
| 3.909 | MMTRA1B | PLSCR1 | phospholipid scramblase 1 |
| 3.792 | XAF1; BIRC4BP; | BIRC4BP | XIAP associated factor-1 |
| | HSXIAPAF1 | | |
| 3.731 | RIGE; SCA2; RIG-E; SCA-2; | LY6E | lymphocyte antigen 6 complex, locus E |
| | TSA-1 | | |
| 3.726 | C7; IFI10; INP10; IP-10; crg-2; | CXCL10 | chemokine (C—X—C motif) ligand 10 |
| | mob-1; SCYB10; gIP-10 | | |
| 3.668 | FBG2; FBS2; FBX6; Fbx6b | FBXO6 | F-box protein 6 |
| 3.652 | RNF94; STAF50; GPSTAF50 | TRIM22 | tripartite motif-containing 22 |
| 3.619 | LOC129607 | LOC129607 | hypothetical protein LOC129607 |
| 3.419 | ISGF-3; STAT91; | STAT1 | signal transducer and activator of |
| | DKFZp686B04100 | | transcription 1, 91 kDa |
| 3.398 | TRIP14; p59OASL | OASL | 2′-5′-oligoadenylate synthetase-like |
| 3.284 | IFP35; FLJ21753 | IFI35 | interferon-induced protein 35 |
| 3.154 | LOC26010; DNAPTP6; | DNAPTP6 | viral DNA polymerase-transactivated |
| | DKFZp564A2416 | | protein 6 |
| 3.076 | BAL; BAL1; FLJ26637; | PARP9 | poly (ADP-ribose) polymerase family, |
| | FLJ41418; MGC: 7868; | | member 9 |
| | DKFZp666B0810; | | |
| | DKFZp686M15238 | | |
| 3.032 | BAL2; KIAA1268 | PARP14 | poly (ADP-ribose) polymerase family, |
| | | | member 14 |
| 2.977 | RIG-B; UBCH8; MGC40331 | UBE2L6 | ubiquitin-conjugating enzyme E2L 6 |
| 2.839 | APT1; PSF1; ABC17; ABCB2; | TAP1 | transporter 1, ATP-binding cassette, sub- |
| | RING4; TAP1N; D6S114E; | | family B (MDR/TAP) |
| | FLJ26666; FLJ41500; | | |
| | TAP1*0102N | | |
| 2.814 | MX; MxA; IFI78; IFI-78K | MX1 | myxovirus (influenza virus) resistance 1, |
| | | | interferon-inducible protein p78 (mouse) |
| 2.632 | | IRF7 | |
| 2.511 | GCH; DYT5; GTPCH1; GTP- | GCH1 | GTP cyclohydrolase 1 (dopa-responsive |
| | CH-1 | | dystonia) |
| 2.434 | 9-27; CD225; IFI17; LEU13 | IFITM1 | interferon induced transmembrane protein 1 |
| | | | (9-27) |
| 2.415 | G10P2; IFI54; ISG54; cig42; | IFIT2 | interferon-induced protein with |
| | IFI-54; GARG-39; ISG-54K | | tetratricopeptide repeats 2 |
| 2.414 | Hlcd; MDA5; MDA-5; | IFIH1 | interferon induced with helicase C domain 1 |
| | IDDM19; MGC133047 | | |
| 2.378 | P113; ISGF-3; STAT113; | STAT2 | signal transducer and activator of |
| | MGC59816 | | transcription 2, 113 kDa |
| 2.321 | TL2; APO2L; CD253; TRAIL; | TNFSF10 | tumor necrosis factor (ligand) superfamily, |
| | Apo-2L | | member 10 |
| 2.32 | TEL2; TELB; TEL-2 | ETV7 | ets variant gene 7 (TEL2 oncogene) |
| 2.214 | OIAS; IFI-4; OIASI | OAS1 | 2′,5′-oligoadenylate synthetase 1, 40/46 kDa |
| 2.206 | APT2; PSF2; ABC18; ABCB3; | TAP2 | transporter 2, ATP-binding cassette, sub- |
| | RING11; D6S217E | | family B (MDR/TAP) |
| 2.134 | MGC78578 | OAS2 | 2′-5′-oligoadenylate synthetase 2, 69/71 kDa |
| 2 | VRK2 | VRK2 | vaccinia related kinase 2 |
| 1.975 | PN-I; PSN1; UMPH; UMPH1; | NT5C3 | 5′-nucleotidase, cytosolic III |
| | P5′N-1; cN-III; MGC27337; | | |
| | MGC87109; MGC87828 | | |
| 1.895 | RNF88; TRIM5alpha | TRIM5 | tripartite motif-containing 5 |
| 1.89 | CGI-34; PNAS-2; C9orf83; | CHMP5 | chromatin modifying protein 5 |
| | HSPC177; SNF7DC2 | | |
| 1.863 | ZC3H1; PARP-12; ZC3HDC1; | PARP12 | poly (ADP-ribose) polymerase family, |
| | FLJ22693 | | member 12 |
| 1.845 | PKR; PRKR; EIF2AK1; | EIF2AK2 | eukaryotic translation initiation factor 2- |
| | MGC126524 | | alpha kinase 2 |
| 1.842 | 90K; MAC-2-BP | LGALS3BP | lectin, galactoside-binding, soluble, 3 |
| | | | binding protein |
| 1.807 | RNF88; TRIM5alpha | TRIM5 | tripartite motif-containing 5 |
| 1.743 | C15; onzin | PLAC8 | placenta-specific 8 |
| 1.732 | p48; IRF9; IRF-9; ISGF3 | ISGF3G | interferon-stimulated transcription factor 3, |
| | | | gamma 48 kDa |
| 1.713 | CD317 | BST2 | bone marrow stromal cell antigen 2 |
| 1.665 | ESNA1; ERAP140; FLJ45605; | NCOA7 | nuclear receptor coactivator 7 |
| | MGC88425; Nbla00052; | | |
| | Nbla10993; dJ187J11.3 | | |
| 1.649 | FLJ39275; MGC131926 | ZNFX1 | zinc finger, NFX1-type containing 1 |
| 1.628 | VODI; IFI41; IFI75; FLJ22835 | SP110 | SP110 nuclear body protein |
| 1.627 | EFP; Z147; RNF147; ZNF147 | TRIM25 | tripartite motif-containing 25 |
| 1.523 | NMI | NMI | N-myc (and STAT) interactor |
| 1.505 | TRAP; KIAA1529; | TDRD7 | tudor domain containing 7 |
| | PCTAIRE2BP; RP11- | | |
| | 508D10.1 | | |
| 1.499 | DSH; G1P1; IFI4; p136; | ADAR | adenosine deaminase, RNA-specific |
| | ADAR1; DRADA; DSRAD; | | |
| | IFI-4; K88dsRBP | | |
| 1.494 | C1GALT; T-synthase | C1GALT1 | core 1 synthase, glycoprotein-N- |
| | | | acetylgalactosamine 3-beta- |
| | | | galactosyltransferase, 1 |
| 1.478 | | PHF11 | |
| 1.461 | SCOTIN | SCOTIN | scotin |
| 1.433 | FLJ00340; FLJ34579; | SP100 | SP100 nuclear antigen |
| | DKFZp686E07254 | | |
| 1.415 | FLJ45064 | AGRN | agrin |
| 1.351 | NFTC; OEF1; OEF2; C7orf5; | SAMD9 | sterile alpha motif domain containing 9 |
| | FLJ20073; KIAA2004 | | |
| 1.26 | MEL; RAB8 | RAB8A | RAB8A, member RAS oncogene family |
| 1.215 | 6-16; G1P3; FAM14C; IFI616; | G1P3 | interferon, alpha-inducible protein 6 |
| | IFI-6-16 | | |

TABLE 7J
M3.2 PTB v. Control, Genes Overrepresented in Active TB.

| Relative | | | |
|---|---|---|---|
| normalised | | | |
| expression | Common Name | Gene Symbol | Description |
| | P22_15_PTBvCSelect_09May08_PAL2Ttest_UP_M3.2 | | |
| 2.767 | MGC20461 | OSM | oncostatin M |
| 2.202 | FHL4; HLH4; HPLH4 | STX11 | syntaxin 11 |
| 2.136 | LPCAT2; FLJ20481; | AYTL1 | acyltransferase like 1 |
| | LysoPAFAT; | | |
| | DKFZp686H22112 | | |
| 1.987 | UP; UPP; UPASE; UDRPASE | UPP1 | uridine phosphorylase 1 |
| 1.969 | IL-1; IL1F2; IL1-BETA | IL1B | interleukin 1, beta |
| 1.886 | SAT; DC21; KFSD; SSAT; | SAT | spermidine/spermine N1-acetyltransferase 1 |
| | SSAT-1 | | |
| 1.862 | PFK2; IPFK2 | PFKFB3 | 6-phosphofructo-2-kinase/fructose-2,6- |
| | | | biphosphatase 3 |
| 1.755 | BB2; CD54; P3.58 | ICAM1 | intercellular adhesion molecule 1 (CD54), |
| | | | human rhinovirus receptor |
| 1.742 | BCL4; D19S37 | BCL3 | B-cell CLL/lymphoma 3 |
| 1.695 | KRML; MGC43127 | MAFB | v-maf musculoaponeurotic fibrosarcoma |
| | | | oncogene homolog B (avian) |
| 1.686 | SRPSOX; CXCLG16; SR- | CXCL16 | chemokine (C—X—C motif) ligand 16 |
| | PSOX | | |
| 1.658 | B3GN-T5; beta3Gn-T5 | B3GNT5 | UDP-GlcNAc:betaGal beta-1,3-N- |
| | | | acetylglucosaminyltransferase 5 |
| 1.62 | MLA1; ME491; LAMP-3; | CD63 | CD63 molecule |
| | OMA81H; TSPAN30 | | |
| 1.562 | P21; CIP1; SDI1; WAF1; | CDKN1A | cyclin-dependent kinase inhibitor 1A (p21, |
| | CAP20; CDKN1; MDA-6; | | Cip1) |
| | p21CIP1 | | |
| 1.548 | URAX1; TAIP-3; FAM130B; | AXUD1 | AXIN1 up-regulated 1 |
| | DKFZp566F164 | | |
| 1.542 | NHE8; FLJ42500; KIAA0939; | SLC9A8 | solute carrier family 9 (sodium/hydrogen |
| | MGC138418; | | exchanger), member 8 |
| | DKFZp686C03237 | | |
| 1.542 | GS; GLNS; PIG43 | GLUL | glutamate-ammonia ligase (glutamine |
| | | | synthetase) |
| 1.504 | CD87; UPAR; URKR | PLAUR | plasminogen activator, urokinase receptor |
| 1.474 | PBEF; NAMPT; MGC117256; | PBEF1 | pre-B-cell colony enhancing factor 1 |
| | DKFZP666B131; | | |
| | 1110035O14Rik | | |
| 1.472 | P47; FLJ27168 | PLEK | pleckstrin |
| 1.45 | GNA16 | GNA15 | guanine nucleotide binding protein (G |
| | | | protein), alpha 15 (Gq class) |
| 1.435 | FTH; PLIF; FTHL6; PIG15; | FTH1 | ferritin, heavy polypeptide 1 |
| | MGC104426 | | |
| 1.42 | MGC14376; MGC149751; | MGC14376 | hypothetical protein MGC14376 |
| | DKFZp686O06159 | | |
| 1.395 | NER; UNR; LXRB; LXR-b; | NR1H2 | nuclear receptor subfamily 1, group H, |
| | NER-I; RIP15 | | member 2 |
| 1.39 | TTP; G0S24; GOS24; TIS11; | ZFP36 | zinc finger protein 36, C3H type, homolog |
| | NUP475; RNF162A | | (mouse) |
| 1.389 | E4BP4; IL3BP1; NFIL3A; NF- | NFIL3 | nuclear factor, interleukin 3 regulated |
| | IL3A | | |
| 1.328 | C8FW; GIG2; SKIP1 | TRIB1 | tribbles homolog 1 (Drosophila) |
| 1.296 | ARI; HARI; HHARI; | ARIH1 | ariadne homolog, ubiquitin-conjugating |
| | UBCH7BP | | enzyme E2 binding protein, 1 (Drosophila) |
| 1.272 | FRA2; FLJ23306 | FOSL2 | FOS-like antigen 2 |
| 1.269 | RIT; RIBB; ROC1; | RIT1 | Ras-like without CAAX 1 |
| | MGC125864; MGC125865 | | |
| 1.25 | RBT1 | SERTAD3 | SERTA domain containing 3 |
| 1.227 | MAPKAPK2 | MAPKAPK2 | mitogen-activated protein kinase-activated |
| | | | protein kinase 2 |
| 1.217 | PPG; PRG; PRG1; MGC9289; | PRG1 | serglycin |
| | FLJ12930 | | |
| 1.181 | SEI1; TRIP-Br1 | SERTAD1 | SERTA domain containing 1 |
| 1.172 | CMT2; KIAA0110; | MAD2L1BP | MAD2L1 binding protein |
| | MGC11282; RP1-261G23.6 | | |
| 1.169 | UBP; SIH003; MGC129878; | USP3 | ubiquitin specific peptidase 3 |
| | MGC129879 | | |

TABLE 7K
M3.3 PTB v. Control, Genes Overrepresented in Active TB.

| Relative | | | |
|---|---|---|---|
| normalised | | | |
| expression | Common Name | Gene Symbol | Description |
| | P22_15_PTBvCSelect_09May08_PAL2Ttest_UP_M3.3 | | |
| 3.651 | MAYP; MGC34175 | PSTPIP2 | proline-serine-threonine phosphatase |
| | | | interacting protein 2 |
| 3.2 | Tiff66; MGC116930; | VNN1 | vanin 1 |
| | MGC116931; MGC116932; | | |
| | MGC116933 | | |
| 2.604 | Rsc6p; BAF60C; CRACD3; | SMARCD3 | SWI/SNF related, matrix associated, actin |
| | MGC111010 | | dependent regulator of chromatin, subfamily |
| | | | d, member 3 |
| 2.157 | FER1L1; LGMD2B; | DYSF | dysferlin, limb girdle muscular dystrophy |
| | FLJ00175; FLJ90168 | | 2B (autosomal recessive) |
| 2.091 | ASRT5; IRAKM; IRAK-M | IRAK3 | interleukin-1 receptor-associated kinase 3 |
| 2.082 | p6; CAGC; CGRP; MRP6; | S100A12 | S100 calcium binding protein A12 |
| | CAAF1; ENRAGE | | |
| 1.888 | CGI-44 | SQRDL | sulfide quinone reductase-like (yeast) |
| 1.819 | FAM31A; FLJ38464; | DENND1A | DENN/MADD domain containing 1A |
| | KIAA1608; RP11-230L22.3 | | |
| 1.736 | APG3; APG3L; PC3-96; | ATG3 | ATG3 autophagy related 3 homolog |
| | FLJ22125; MGC15201; | | (S. cerevisiae) |
| | DKFZp564M1178 | | |
| 1.715 | CAT1 | CRAT | carnitine acetyltransferase |
| 1.703 | MGC2654; FLJ12433 | MGC2654 | chromosome 16 open reading frame 68 |
| 1.7 | MD-2 | LY96 | lymphocyte antigen 96 |
| 1.695 | AD3; VRP; HBLP1 | TBC1D8 | TBC1 domain family, member 8 (with |
| | | | GRAM domain) |
| 1.663 | FLJ20424 | C14or194 | chromosome 14 open reading frame 94 |
| 1.638 | P28; GSTTLp28; | GSTO1 | glutathione S-transferase omega 1 |
| | DKFZp686H13163 | | |
| 1.635 | ATRAP; MGC29646 | AGTRAP | angiotensin II receptor-associated protein |
| 1.572 | FAT; GP4; GP3B; GPIV; | CD36 | CD36 molecule (thrombospondin receptor) |
| | CHDS7; PASIV; SCARB3 | | |
| 1.547 | EI; LEI; PI2; MNEI; M/NEI; | SERPINB1 | serpin peptidase inhibitor, clade B |
| | ELANH2 | | (ovalbumin), member 1 |
| 1.546 | RAB32 | RAB32 | RAB32, member RAS oncogene family |
| 1.541 | CR3A; MO1A; CD11B; MAC- | ITGAM | integrin, alpha M (complement component 3 |
| | 1; MAC1A; MGC117044 | | receptor 3 subunit) |
| 1.481 | ALFY; ZFYVE25; KIAA0993; | WDFY3 | WD repeat and FYVE domain containing 3 |
| | MGC16461 | | |
| 1.467 | ARHU; WRCH1; hG28K; | RHOU | ras homolog gene family, member U |
| | CDC42L1; FLJ10616; | | |
| | DJ646B12.2; fJ646B12.2 | | |
| 1.459 | SELR; SELX; MSRB1; | SEPX1 | selenoprotein X, 1 |
| | HSPC270; MGC3344 | | |
| 1.432 | LTA4H | LTA4H | leukotriene A4 hydrolase |
| 1.409 | VMP1; DKFZP566I133 | TMEM49 | transmembrane protein 49 |
| 1.405 | MGC33054 | SNX10 | sorting nexin 10 |
| 1.376 | STX3A | STX3A | syntaxin 3 |
| 1.369 | TTG2; RBTN2; RHOM2; | LMO2 | LIM domain only 2 (rhombotin-like 1) |
| | RBTNL1 | | |
| 1.368 | DBI; IBP; MBR; PBR; BZRP; | BZRP | translocator protein (18 kDa) |
| | PKBS; PTBR; mDRC; pk18 | | |
| 1.361 | CRE-BPA | CREB5 | cAMP responsive element binding protein 5 |
| 1.344 | MAY1; MGC49908; nPKC- | PRKCD | protein kinase C, delta |
| | delta | | |
| 1.341 | AAA; AD1; PN2; ABPP; | APP | amyloid beta (A4) precursor protein |
| | APPI; CVAP; ABETA; | | (peptidase nexin-II, Alzheimer disease) |
| | CTFgamma | | |
| 1.333 | CRFB4; CRF2-4; D21S58; | IL10RB | interleukin 10 receptor, beta |
| | D21S66; CDW210B; IL-10R2 | | |
| 1.31 | DCIR; LLIR; DDB27; | CLEC4A | C-type lectin domain family 4, member A |
| | CLECSF6; HDCGC13P | | |
| 1.304 | HUFI-2; FLJ20248; FLJ22683; | LRRFIP2 | leucine rich repeat (in FLII) interacting |
| | DKFZp434H2035 | | protein 2 |
| 1.301 | C32; CKLF1; CKLF2; CKLF3; | CKLF | chemokine-like factor |
| | CKLF4; UCK-1; HSPC224 | | |
| 1.289 | | ACSS2 | |
| 1.265 | ESP-2; HED-2 | ZYX | zyxin |
| 1.263 | SH3BGR; MGC117402 | SH3BGRL | SH3 domain binding glutamic acid-rich |
| | | | protein like |
| 1.239 | MTX; MTXN | MTX1 | metaxin 1 |
| 1.237 | ASC; TMS1; CARD5; | PYCARD | PYD and CARD domain containing |
| | MGC10332 | | |
| 1.233 | a3; Stv1; Vph1; Atp6i; OC116; | TCIRG1 | T-cell, immune regulator 1, ATPase, H+ |
| | OPTB1; TIRC7; ATP6N1C; | | transporting, lysosomal V0 subunit A3 |
| | ATP6V0A3; OC-116 kDa | | |
| 1.223 | JTK8; FLJ26625 | LYN | v-yes-1 Yamaguchi sarcoma viral related |
| | | | oncogene homolog |
| 1.209 | GAIP; RGSGAIP | RGS19 | regulator of G-protein signalling 19 |
| 1.186 | NEU; SIAL1 | NEU1 | sialidase 1 (lysosomal sialidase) |

TABLE 7L
M3.4 PTB v. Control, Genes Underrepresented in Active TB

| Relative | | | |
|---|---|---|---|
| normalised | | | |
| expression | Common Name | Gene Symbol | Description |
| | P22_15_PTBvCSelect_09May08_PAL2Ttest_DOWN_M3.4 | | |
| 0.921 | ZZZ4; FLJ10821; FLJ45574; | ZZEF1 | zinc finger, ZZ-type with EF-hand domain 1 |
| | KIAA0399 | | |
| 0.905 | TILZ4a; TILZ4b; TILZ4c; | TSC22D2 | TSC22 domain family, member 2 |
| | KIAA0669 | | |
| 0.891 | XTP2; BAT2-iso | BAT2D1 | BAT2 domain containing 1 |
| 0.885 | U2AF65 | U2AF2 | U2 small nuclear RNA auxiliary factor 2 |
| 0.878 | DKFZp781I24156 | PCNP | PEST proteolytic signal containing nuclear |
| | | | protein |
| 0.876 | NY-CO-1; FLJ10051 | SDCCAG1 | serologically defined colon cancer antigen 1 |
| 0.868 | GCP16; HSPC041; MGC4876; | GOLGA7 | golgi autoantigen, golgin subfamily a, 7 |
| | MGC21096; GOLGA3AP1 | | |
| 0.866 | CPR3; DJA2; DNAJ; DNJ3; | DNAJA2 | DnaJ (Hsp40) homolog, subfamily A, |
| | RDJ2; HIRIP4; PRO3015 | | member 2 |
| 0.863 | B2-1; SEC7; D17S811E; | PSCD1 | pleckstrin homology, Sec7 and coiled-coil |
| | FLJ34050; FLJ41900; | | domains 1(cytohesin 1) |
| | CYTOHESIN-1 | | |
| 0.855 | SRrp86; SRrp508; | SFRS12 | splicing factor, arginine/serine-rich 12 |
| | MGC133045; DKFZp564B176 | | |
| 0.84 | G3BP2 | G3BP2 | GTPase activating protein (SH3 domain) |
| | | | binding protein 2 |
| 0.831 | p532; p619 | HERC1 | hect (homologous to the E6-AP (UBE3A) |
| | | | carboxyl terminus) domain and RCC1 |
| | | | (CHC1)-like domain (RLD) 1 |
| 0.826 | DKFZP564O0523; HSPC304; | DKFZP564O0523 | hypothetical protein DKFZp564O0523 |
| | DKFZp686D1651 | | |
| 0.823 | TSPYL | TSPYL1 | TSPY-like 1 |
| 0.82 | KIP1; MEN4; CDKN4; | CDKN1B | cyclin-dependent kinase inhibitor 1B (p27, |
| | MEN1B; P27KIP1 | | Kip1) |
| 0.82 | SA2; SA-2; FLJ25871; | STAG2 | stromal antigen 2 |
| | bA517O1.1; DKFZp686P168; | | |
| | DKFZp781H1753 | | |
| 0.815 | HR21; MCD1; NXP1; SCC1; | RAD21 | RAD21 homolog (S. pombe) |
| | hHR21; HRAD21; FLJ25655; | | |
| | FLJ40596; KIAA0078 | | |
| 0.808 | GCC185; KIAA0336 | GCC2 | GRIP and coiled-coil domain containing 2 |
| 0.806 | PIR1 | DUSP11 | dual specificity phosphatase 11 (RNA/RNP |
| | | | complex 1-interacting) |
| 0.804 | AS3; CG008; PDS5B; | APRIN | androgen-induced proliferation inhibitor |
| | FLJ23236; KIAA0979; RP1- | | |
| | 267P19.1 | | |
| 0.803 | | LOC58486 | |
| 0.798 | | SLTM | |
| 0.795 | AS; ANCR; E6-AP; HPVE6A; | UBE3A | ubiquitin protein ligase E3A (human |
| | EPVE6AP; FLJ26981 | | papilloma virus E6-associated protein, |
| | | | Angelman syndrome) |
| 0.793 | DKFZp686C1054 | THUMPD1 | THUMP domain containing 1 |
| 0.791 | SIR2L1 | SIRT1 | sirtuin (silent mating type information |
| | | | regulation 2 homolog) 1 (S. cerevisiae) |
| 0.79 | FLJ40359 | TPP2 | tripeptidyl peptidase II |
| 0.789 | DKFZP564D172 | C5orf21 | chromosome 5 open reading frame 21 |
| 0.788 | PALBH; CALPAIN7; | CAPN7 | calpain 7 |
| | FLJ36423 | | |
| 0.775 | KIAA1116 | RBM16 | RNA binding motif protein 16 |
| 0.771 | FLJ42355; KIAA0276 | DCUN1D4 | DCN1, defective in cullin neddylation 1, |
| | | | domain containing 4 (S. cerevisiae) |
| 0.768 | Rhe; FLJ33619; | FIP1L1 | FIP1 like 1 (S. cerevisiae) |
| | DKFZp586K0717 | | |
| 0.766 | RCP9; RCP; CRCP; CGRP- | RCP9 | calcitonin gene-related peptide-receptor |
| | RCP; MGC111194 | | component protein |
| 0.764 | DIF3; LZK1; DIF-3; LCRG1; | ZNF403 | zinc finger protein 403 |
| | ZFP403; FLJ21230; FLJ22561; | | |
| | FLJ42090 | | |
| 0.76 | AD013; CReMM; KISH2; | CHD9 | chromodomain helicase DNA binding |
| | PRIC320 | | protein 9 |
| 0.757 | VACM1; VACM-1 | CUL5 | cullin 5 |
| 0.755 | MGC13407 | NUP54 | nucleoporin 54 kDa |
| 0.751 | ENTH; EPN4; EPNR; CLINT; | ENTH | clathrin interactor 1 |
| | EPSINR; KIAA0171 | | |
| 0.743 | SEC24B | SEC24B | SEC24 related gene family, member B; |
| | | | (S. cerevisiae) synonyms: |
| | | | SEC24, MGC48822; |
| | | | isoform a is encoded by transcript variant 1; |
| | | | secretory protein 24; Sec24-related protein |
| | | | B; protein transport protein Sec24B; Homo |
| | | | sapiens SEC24 related gene family, member |
| | | | B (S. cerevisiae) (SEC24B), transcript |
| | | | variant 1, mRNA. |
| 0.742 | HAKAI; RNF188; FLJ23109; | CBLL1 | Cas-Br-M (murine) ecotropic retroviral |
| | MGC163401; MGC163403 | | transforming sequence-like 1 |
| 0.738 | XE7; 721P; XE7Y; CCDC133; | DXYS155E | splicing factor, arginine/serine-rich 17A |
| | CXYorf3; DXYS155E; | | |
| | MGC39904; MGC125365; | | |
| | MGC125366 | | |
| 0.737 | NGB; CRFG; FLJ10686; | GTPBP4 | GTP binding protein 4 |
| | FLJ10690; FLJ39774 | | |
| 0.734 | VELI3; LIN-7C; MALS-3; | LIN7C | lin-7 homolog C (C. elegans) |
| | LIN-7-C; FLJ11215 | | |
| 0.732 | JTK5; RYK1; JTK5A; | RYK | RYK receptor-like tyrosine kinase |
| | D3S3195 | | |
| 0.731 | K10; KPP; CK10 | KRT10 | keratin 10 (epidermolytic hyperkeratosis; |
| | | | keratosis palmaris et plantaris) |
| 0.728 | CYP-M; MGC22229 | CYP20A1 | cytochrome P450, family 20, subfamily A, |
| | | | polypeptide 1 |
| 0.725 | CHP1 | CHORDC1 | cysteine and histidine-rich domain |
| | | | (CHORD)-containing 1 |
| 0.724 | NET1A; ARHGEF8 | NET1 | neuroepithelial cell transforming gene 1 |
| 0.723 | ZF5; ZBTB14; ZNF478; | ZFP161 | zinc finger protein 161 homolog (mouse) |
| | MGC126126 | | |
| 0.718 | JAK1A; JAK1B | JAK1 | Janus kinase 1 (a protein tyrosine kinase) |
| 0.717 | p5; p6; RRP45; PMSCL1; | EXOSC9 | exosome component 9 |
| | Rrp45p; PM/Scl-75 | | |
| 0.716 | GR; GCR; GRL; GCCR | NR3C1 | nuclear receptor subfamily 3, group C, |
| | | | member 1 (glucocorticoid receptor) |
| 0.713 | L9mt | MRPL9 | mitochondrial ribosomal protein L9 |
| 0.705 | GRB1; p85-ALPHA | PIK3R1 | phosphoinositide-3-kinase, regulatory |
| | | | subunit 1 (p85 alpha) |
| 0.7 | MST4; MASK | MASK | serine/threonine protein kinase MST4 |
| 0.7 | UPF3; HUPF3A; RENT3A | UPF3A | UPF3 regulator of nonsense transcripts |
| | | | homolog A (yeast) |
| 0.698 | p17; YBL1; CHRAC17; | POLE3 | polymerase (DNA directed), epsilon 3 (p17 |
| | CHARAC17 | | subunit) |
| 0.694 | PCGF4; RNF51; MGC12685 | PCGF4 | BMI1 polycomb ring finger oncogene |
| 0.692 | MIF2; CENPC; hcp-4; CENP-C | CENPC1 | centromere protein C 1 |
| 0.686 | YAF9; GAS41; NUBI-1; | YEATS4 | YEATS domain containing 4 |
| | 4930573H17Rik; | | |
| | B230215M10Rik | | |
| 0.679 | R3HDM; FLJ23334; | R3HDM1 | R3H domain containing 1 |
| | KIAA0029 | | |
| 0.676 | FBX21; FLJ90233; KIAA0875; | FBXO21 | F-box protein 21 |
| | MGC26682; DKFZp434G058 | | |
| 0.665 | GRIPE; TULIP1; KIAA0884; | GARNL1 | GTPase activating Rap/RanGAP domain- |
| | DKFZp566D133; | | like 1 |
| | DKFZp667F074 | | |
| 0.663 | BRL; BRPF1; BRPF2; | BRD1 | bromodomain containing 1 |
| | DKFZp686F0325 | | |
| 0.651 | TIFIA; MGC104238; | RRN3 | RRN3 RNA polymerase I transcription |
| | DKFZp566E104 | | factor homolog (S. cerevisiae) |
| 0.65 | DKFZP586L0724 | NOL11 | nucleolar protein 11 |
| 0.645 | FLJ20628; DKFZp564I2178 | FLJ20628 | hypothetical protein FLJ20628 |
| 0.642 | FLJ21657; MGC90226; | FLJ21657 | chromosome 5 open reading frame 28 |
| | MGC149524 | | |
| 0.638 | NS3TP1; FLJ20752; | ASNSD1 | asparagine synthetase domain containing 1 |
| | NBLA00058 | | |
| 0.636 | MEX3C; BM-013; MEX-3C; | RKHD2 | ring finger and KH domain containing 2 |
| | RNF194; FLJ38871 | | |
| 0.628 | E6BP; ERC55; ERC-55 | RCN2 | reticulocalbin 2, EF-hand calcium binding |
| | | | domain |
| 0.613 | PHLL1 | CRY1 | cryptochrome 1 (photolyase-like) |
| 0.612 | cdc14; hCDC14; Cdc14A1; | CDC14A | CDC14 cell division cycle 14 homolog A |
| | Cdc14A2 | | (S. cerevisiae) |
| 0.576 | LCA; LY5; B220; CD45; | PTPRC | protein tyrosine phosphatase, receptor type, C |
| | T200; GP180 | | |
| 0.521 | PBF; PRF1; HDBP2; PRF-1; | ZNF395 | zinc finger protein 395 |
| | Si-1-8-14; DKFZp434K1210 | | |

TABLE 7M
M3.6 PTB v. Control, Genes Underrepresented in Active TB.

| Relative | | | |
|---|---|---|---|
| normalised | | | |
| expression | Common Name | Gene Symbol | Description |
| | P22_15_PTBvCSelect_09May08_PAL2Ttest_DOWN_M3.6 | | |
| 0.898 | ABHS; ORF20; TTDN1 | C7orf11 | chromosome 7 open reading frame 11 |
| 0.852 | BTF2; TFIIH | GTF2H1 | general transcription factor IIH, polypeptide |
| | | | 1, 62 kDa |
| 0.845 | MGC51029 | FUNDC1 | FUN14 domain containing 1 |
| 0.844 | SCOCO; HRIHFB2072 | SCOC | short coiled-coil protein |
| 0.839 | IF-3mt; IF3(mt) | MTIF3 | mitochondrial translational initiation factor 3 |
| 0.816 | DAB1; MPRP-1; YKR087C; | OMA1 | OMA1 homolog, zinc metallopeptidase (S. cerevisiae) |
| | ZMPOMA1; FLJ33782; | | |
| | 2010001O09Rik | | |
| 0.815 | | LOC644560 | |
| 0.795 | JNKK; MEK4; MKK4; SEK1; | MAP2K4 | mitogen-activated protein kinase kinase 4 |
| | JNKK1; SERK1; MAPKK4; | | |
| | PRKMK4 | | |
| 0.775 | REPA2; RPA32 | RPA2 | replication protein A2, 32 kDa |
| 0.765 | AMMERC1 | AMMECR1 | Alport syndrome, mental retardation, |
| | | | midface hypoplasia and elliptocytosis |
| | | | chromosomal region, gene 1 |
| 0.741 | CBX; M31; MOD1; HP1- | CBX1 | chromobox homolog 1 (HP1 beta homolog |
| | BETA; HP1Hs-beta | | Drosophila) |
| 0.739 | DLTA; PDCE2; PDC-E2 | DLAT | dihydrolipoamide S-acetyltransferase (E2 |
| | | | component of pyruvate dehydrogenase |
| | | | complex) |
| 0.732 | p38; AHA1; C14orf3 | AHSA1 | AHA1, activator of heat shock 90 kDa |
| | | | protein ATPase homolog 1 (yeast) |
| 0.731 | VEZATIN; DKFZp761C241 | VEZT | vezatin, adherens junctions transmembrane |
| | | | protein |
| 0.728 | HDPY-30 | LOC84661 | dpy-30-like protein |
| 0.727 | DERP6; MST071; HSPC002; | C17orf81 | chromosome 17 open reading frame 81 |
| | MSTP071 | | |
| 0.723 | EFG; GFM; EFG1; EFGM; | GFM1 | G elongation factor, mitochondrial 1 |
| | EGF1; hEFG1; COXPD1; | | |
| | FLJ12662; FLJ13632; | | |
| | FLJ20773 | | |
| 0.721 | MGC3232; hAtNOS1; | C4orf14 | chromosome 4 open reading frame 14 |
| | mAtNOS1 | | |
| 0.72 | P15RS; FLJ10656; MGC19513 | P15RS | hypothetical protein FLJ10656 |
| 0.719 | MGC9912 | C14orf126 | chromosome 14 open reading frame 126 |
| 0.704 | CCR4; KIAA1194 | CNOT6 | CCR4-NOT transcription complex, subunit 6 |
| 0.7 | PRED31; HSPC230; | C6orf203 | chromosome 6 open reading frame 203 |
| | FLJ34245; RP11-59I9.1 | | |
| 0.696 | 76P; GCP4 | 76P | gamma tubulin ring complex protein (76p |
| | | | gene) |
| 0.694 | FLJ10422 | ELP3 | elongation protein 3 homolog (S. cerevisiae) |
| 0.677 | MGC13379 | MGC13379 | HSPC244 |
| 0.677 | CCTE; KIAA0098; CCT- | CCT5 | chaperonin containing TCP1, subunit 5 |
| | epsilon; TCP-1-epsilon | | (epsilon) |
| 0.675 | | MTMR12 | |
| 0.671 | ABRA1; FLJ11520; FLJ12642; | FLJ13614 | coiled-coil domain containing 98 |
| | FLJ13614 | | |
| 0.671 | CDG1; CDGS; CDG1a | PMM2 | phosphomannomutase 2 |
| 0.646 | TPA1; FLJ10826; KIAA1612 | OGFOD1 | 2-oxoglutarate and iron-dependent |
| | | | oxygenase domain containing 1 |
| 0.641 | HV1; MGC15619 | MGC15619 | hydrogen voltage-gated channel 1 |
| 0.639 | JJJ3; ZCSL3 | ZCSL3 | DPH4, JJJ3 homolog (S. cerevisiae) |
| 0.631 | GI008; RPMS13; MRP-S13; | MRPS26 | mitochondrial ribosomal protein S26 |
| | MRP-S26; NY-BR-87; | | |
| | C20orf193; dJ534B8.3 | | |
| 0.63 | RPMS6; MRP-S6; C21orf101 | MRPS6 | mitochondrial ribosomal protein S6 |
| 0.622 | CGI-55; CHD3IP; HABP4L; | SERBP1 | SERPINE1 mRNA binding protein 1 |
| | PAIRBP1; FLJ90489; PAI- | | |
| | RBP1; DKFZp564M2423 | | |
| 0.621 | MRP-S14; HSMRPS14; | MRPS14 | mitochondrial ribosomal protein S14 |
| | DJ262D12.2 | | |
| 0.542 | LOC153364; MGC46734; | LOC153364 | similar to metallo-beta-lactamase |
| | DKFZp686P15118 | | superfamily protein |

TABLE 7N
M3.7 PTB v. Control, Genes Underrepresented in Active TB.

| Relative | | | |
|---|---|---|---|
| normalised | | | |
| expression | Common Name | Gene Symbol | Description |
| | P22_15_PTBvCSelect_09May08_PAL2Ttest_DOWN_M3.7 | | |
| 0.914 | RED; CSA2; MGC59741; IK | IK | IK cytokine, down-regulator of HLA II |
| | protein | | |
| 0.875 | IBP | DEF6 | differentially expressed in FDCP 6 homolog |
| | | | (mouse) |
| 0.861 | NAT3; dJ1002M8.1 | NAT5 | N-acetyltransferase 5 |
| 0.857 | OFOXD; OFOXD1; FLJ20308 | ALKBH5 | alkB, alkylation repair homolog 5 (E. coli) |
| 0.848 | H-IDHB; MGC903; FLJ11043 | IDH3B | isocitrate dehydrogenase 3 (NAD+) beta |
| 0.846 | PGR1; PAM14 | MRFAP1 | Mof4 family associated protein 1 |
| 0.845 | B17.2; DAP13 | NDUFA12 | NADH dehydrogenase (ubiquinone) 1 alpha |
| | | | subcomplex, 12 |
| 0.836 | MGC11134 | TRPT1 | tRNA phosphotransferase 1 |
| 0.832 | H-l(3)mbt-l | L3MBTL2 | 1(3)mbt-like 2 (Drosophila) |
| 0.831 | HSCARG; FLJ25918 | HSCARG | NmrA-like family domain containing 1 |
| 0.817 | ABC27; ABC50 | ABCF1 | ATP-binding cassette, sub-family F |
| | | | (GCN20), member 1 |
| 0.816 | LOC124512 | LOC124512 | hypothetical protein LOC124512 |
| 0.815 | HSPC203 | C14orf112 | chromosome 14 open reading frame 112 |
| 0.814 | EXOSC1 | EXOSC1 | exosome component 1; synonyms: p13, |
| | | | CSL4, SKI4, Csl4p, Ski4p, hCsl4p, CGI- |
| | | | 108, RP11-452K12.9; homolog of yeast |
| | | | exosomal core protein CSL4; 3′-5′ |
| | | | exoribonuclease CSL4 homolog; CSL4 |
| | | | exosomal core protein homolog; Homo |
| | | | sapiens exosome component 1 (EXOSC1), |
| | | | mRNA. |
| 0.81 | p14; DOC-1R; FLJ10636 | CDK2AP2 | CDK2-associated protein 2 |
| 0.81 | MGC14833; bA6B20.2 | C6orf125 | chromosome 6 open reading frame 125 |
| 0.809 | SRP68 | SRP68 | signal recognition particle 68 kDa |
| 0.805 | MGC3320; FLJ14936; RP5- | PRPF38A | PRP38 pre-mRNA processing factor 38 |
| | 965L7.1 | | (yeast) domain containing A |
| 0.805 | DBP-RB; UKVH5d | DDX1 | DEAD (Asp-Glu-Ala-Asp) box polypeptide 1 |
| 0.804 | ACRP; FSA-1; MGC20134 | SPAG7 | sperm associated antigen 7 |
| 0.802 | MDHA; MOR2; MDH-s; | MDH1 | malate dehydrogenase 1, NAD (soluble) |
| | MGC: 1375 | | |
| 0.801 | MDS016; RPMS21; MRP-S21 | MRPS21 | mitochondrial ribosomal protein S21 |
| 0.8 | AIBP; MGC119143; | APOA1BP | apolipoprotein A-I binding protein |
| | MGC119144; MGC119145 | | |
| 0.8 | ERV29; FLJ22993; | SURF4 | surfeit 4 |
| | MGC102753 | | |
| 0.797 | MGC874 | CXorf26 | chromosome X open reading frame 26 |
| 0.795 | FLJ22789 | C12orf26 | chromosome 12 open reading frame 26 |
| 0.795 | RC68; INT11; RC-68; INTS11; | CPSF3L | cleavage and polyadenylation specific factor |
| | CPSF73L; FLJ13294; | | 3-like |
| | FLJ20542 | | |
| 0.793 | HSPC196 | HSPC196 | transmembrane protein 138 |
| 0.79 | DS-1 | ICT1 | immature colon carcinoma transcript 1 |
| 0.789 | SIAHBP1; FIR; PUF60; | SIAHBP1 | fuse-binding protein-interacting repressor |
| | RoBPI; FLJ31379 | | |
| 0.788 | bMRP36a; MGC17989; | MRPL43 | mitochondrial ribosomal protein L43 |
| | MGC48892 | | |
| 0.788 | HIT-17 | HINT2 | histidine triad nucleotide binding protein 2 |
| 0.785 | MGC2714; FLJ32431 | DCUN1D5 | DCN1, defective in cullin neddylation 1, |
| | | | domain containing 5 (S. cerevisiae) |
| 0.784 | WDC146; FLJ11294 | WDR33 | WD repeat domain 33 |
| 0.775 | N27C7-4; MGC70831 | C22orf16 | chromosome 22 open reading frame 16 |
| 0.774 | | LOC653709 | |
| 0.772 | CGI-138; HSPC329; MRP-S23 | MRPS23 | mitochondrial ribosomal protein S23 |
| 0.769 | P54; NMT55; NRB54; | NONO | non-POU domain containing, octamer- |
| | P54NRB | | binding |
| 0.764 | NSE2; MMS21; C8orf36; | C8orf36 | non-SMC element 2, MMS21 homolog (S. cerevisiae) |
| | FLJ32440 | | |
| 0.764 | C8orf40 | C8orf40 | chromosome 8 open reading frame 40 |
| 0.763 | FLJ31795 | CCDC43 | coiled-coil domain containing 43 |
| 0.755 | NSE1 | NSMCE1 | non-SMC element 1 homolog (S. cerevisiae) |
| 0.753 | MY105; THY28; MDS012; | THYN1 | thymocyte nuclear protein 1 |
| | HSPC144; THY28KD; | | |
| | MGC12187 | | |
| 0.752 | YSA1H; hYSAH1 | NUDT5 | nudix (nucleoside diphosphate linked |
| | | | moiety X)-type motif 5 |
| 0.751 | TOK-1 | BCCIP | BRCA2 and CDKN1A interacting protein |
| 0.747 | VARSL; VARS2L; | VARSL | valyl-tRNA synthetase 2, mitochondrial |
| | MGC138259; MGC142165 | | (putative) |
| 0.732 | FLJ13657; RP11-337A23.1 | C9orf82 | chromosome 9 open reading frame 82 |
| 0.728 | GLOD2 | MCEE | methylmalonyl CoA epimerase |
| 0.728 | C40 | C2orf29 | chromosome 2 open reading frame 29 |
| 0.726 | MGC12966 | MGC12966 | hypothetical protein LOC84792; Homo |
| | | | sapiens hypothetical protein LOC84792 |
| | | | (MGC12966), mRNA. |
| 0.722 | FLJ14803 | FLJ14803 | hypothetical protein FLJ14803 |
| 0.717 | HSPC335; MRP-S24 | MRPS24 | mitochondrial ribosomal protein S24 |
| 0.716 | RALBP1 | REPS1 | RALBP1 associated Eps domain containing 1 |
| 0.712 | CAF1; hCAF-1 | CNOT7 | CCR4-NOT transcription complex, subunit 7 |
| 0.711 | A1U; UBIN; C1orf6 | UBQLN4 | ubiquilin 4 |
| 0.71 | CGI-118; MGC13323 | MRPL48 | mitochondrial ribosomal protein L48 |
| 0.701 | Gm83; HSPC064; | WDSOF1 | WD repeats and SOF1 domain containing |
| | MGC126859; MGC138247; | | |
| | DKFZP564O0463 | | |
| 0.701 | FMT1 | MTFMT | mitochondrial methionyl-tRNA |
| | | | formyltransferase |
| 0.697 | DKFZp686E10109 | NUDCD2 | NudC domain containing 2 |
| 0.697 | MGC11321 | MRPL45 | mitochondrial ribosomal protein L45 |
| 0.691 | SDOS; MGC11275 | NUDT16L1 | nudix (nucleoside diphosphate linked |
| | | | moiety X)-type motif 16-like 1 |
| 0.683 | FLJ20989 | C8orf33 | chromosome 8 open reading frame 33 |
| 0.681 | AK6; FIX; AK3L1; AKL3L; | AK3 | adenylate kinase 3 |
| | AKL3L1 | | |
| 0.671 | RIP; HRIP; MGC4189 | RIP | RPA interacting protein |
| 0.666 | PRP8; RP13; HPRP8; PRPC8 | PRPF8 | PRP8 pre-mRNA processing factor 8 |
| | | | homolog (S. cerevisiae) |
| 0.664 | PCMT; PPMT; PCCMT; | ICMT | isoprenylcysteine carboxyl |
| | HSTE14; MST098; MSTP098; | | methyltransferase |
| | MGC39955 | | |
| 0.66 | YTM1; FLJ10881; FLJ12719; | WDR12 | WD repeat domain 12 |
| | FLJ12720 | | |
| 0.646 | GAB1; CDC91L1; MGC40420 | CDC91L1 | phosphatidylinositol glycan anchor |
| | | | biosynthesis, class U |
| 0.613 | MGC4248 | C10orf58 | chromosome 10 open reading frame 58 |
| 0.613 | sen15 | C1orf19 | chromosome 1 open reading frame 19 |
| 0.599 | MGC2404 | ACBD6 | acyl-Coenz A binding domain containing 6 |

TABLE 7O
M3.8 PTB v. Control, Genes Underrepresented in Active TB.

| Relative | | | |
|---|---|---|---|
| normalised | | Gene | |
| expression | Common Name | Symbol | Description |
| | P22_15_PTBvCSelect_09May08_PAL2Ttest_DOWN_M3.8 | | |
| 0.841 | MAP; RUSC3; SGSM3; | RUTBC3 | RUN and TBC1 domain containing 3 |
| | DKFZp761D051 | | |
| 0.84 | FLJ13848 | FLJ13848 | N-acetyltransferase 11 |
| 0.827 | HEL308; MGC20604 | HEL308 | DNA helicase HEL308 |
| 0.826 | dgkd-2; DGKdelta; KIAA0145 | DGKD | diacylglycerol kinase, delta 130 kDa |
| 0.814 | DKFZp779L2418 | SFRS14 | splicing factor, arginine/serine-rich 14 |
| 0.814 | HMMH; MUTM; OGH1; | OGG1 | 8-oxoguanine DNA glycosylase |
| | HOGG1 | | |
| 0.808 | PRO9856; LAVS3040; | BRD9 | bromodomain containing 9 |
| | DKFZp434D0711; | | |
| | DKFZp686L0539 | | |
| 0.807 | HCDI | C14orf124 | chromosome 14 open reading frame 124 |
| 0.798 | GTF2D; SCA17; TFIID; | TBP | TATA box binding protein |
| | GTF2D1; MGC117320; | | |
| | MGC126054; MGC126055 | | |
| 0.772 | ZIS; ZIS1; ZIS2; ZNF265; | ZNF265 | zinc finger, RAN-binding domain |
| | FLJ41119; DKFZp686J1831; | | containing 2 |
| | DKFZp686N09117 | | |
| 0.764 | | OGT | |
| 0.762 | MTMR8; C8orf9; LIP-STYX; | MTMR9 | myotubularin related protein 9 |
| | MGC126672; DKFZp434K171 | | |
| 0.76 | TDP-43 | TARDBP | TAR DNA binding protein |
| 0.754 | FPM315; ZKSCAN12 | ZNF263 | zinc finger protein 263 |
| 0.754 | C42; CGI-05; HSPC167; | CDK5RAP1 | CDK5 regulatory subunit associated |
| | C20orf34; CDK5RAP1.3; | | protein 1 |
| | CDK5RAP1.4 | | |
| 0.747 | P50; P85; PAK3; PIXB; | ARHGEF7 | Rho guanine nucleotide exchange factor |
| | COOL1; P50BP; P85SPR; | | (GEF) 7 |
| | BETA-PIX; KIAA0142; | | |
| | KIAA0412; P85COOL1; | | |
| | Nbla10314; DKFZp761K1021 | | |
| 0.745 | NAC; CARD7; NALP1; | NALP1 | NLR family, pyrin domain containing 1 |
| | SLEV1; DEFCAP; PP1044; | | |
| | VAMAS1; CLR17.1; | | |
| | KIAA0926; DEFCAP-L/S; | | |
| | DKFZp586O1822 | | |
| 0.744 | KIAA0388 | EZH1 | enhancer of zeste homolog 1 (Drosophila) |
| 0.741 | MGC19570; dJ34B21.3 | C6orf130 | chromosome 6 open reading frame 130 |
| 0.737 | RP11-336K24.1 | KIAA0907 | KIAA0907 |
| 0.732 | LAM; TSC; KIAA0243; | TSC1 | tuberous sclerosis 1 |
| | MGC86987 | | |
| 0.725 | LRS; LEUS; LARS1; LEURS; | LARS | leucyl-tRNA synthetase |
| | PIG44; RNTLS; HSPC192; | | |
| | hr025Cl; FLJ10595; FLJ21788; | | |
| | KIAA1352 | | |
| 0.724 | HZF1 | ZNF266 | zinc finger protein 266 |
| 0.72 | FAC1; FALZ; NURF301 | FALZ | bromodomain PHD finger transcription |
| | | | factor |
| 0.72 | FLJ12892; FLJ41065; | CCDC14 | coiled-coil domain containing 14 |
| | DKFZp434L1050 | | |
| 0.708 | TIR8; MGC110992 | SIGIRR | single immunoglobulin and toll-interleukin |
| | | | 1 receptor (TIR) domain |
| 0.7 | FLJ21007; RP11-459E2.1 | TDRD3 | tudor domain containing 3 |
| 0.691 | CGI75; mtTFB; CGI-75 | TFB1M | transcription factor B1, mitochondrial |
| 0.689 | FP977; FLJ12270; MGC11230 | WDR59 | WD repeat domain 59 |
| 0.684 | TS11 | ASNS | asparagine synthetase |
| 0.677 | MGC111199 | NIT2 | nitrilase family, member 2 |
| 0.675 | | ASB1 | |
| 0.663 | MCAF2; FLJ12668 | ATF7IP2 | activating transcription factor 7 interacting |
| | | | protein 2 |
| 0.648 | SIN; RPC5 | POLR3E | polymerase (RNA) III (DNA directed) |
| | | | polypeptide E (80 kD) |
| 0.646 | BMS1L; KIAA0187 | BMS1L | BMS1 homolog, ribosome assembly |
| | | | protein (yeast) |
| 0.636 | CBX7 | CBX7 | chromobox homolog 7 |
| 0.63 | PAN2; hPAN2; FLJ39360; | USP52 | ubiquitin specific peptidase 52 |
| | KIAA0710 | | |
| 0.623 | MSK1; RLPK; MSPK1; | RPS6KA5 | ribosomal protein S6 kinase, 90 kDa, |
| | MGC1911 | | polypeptide 5 |
| 0.612 | SYB1; VAMP-1; | VAMP1 | vesicle-associated membrane protein 1 |
| | DKFZp686H12131 | | (synaptobrevin 1) |
| 0.601 | ALC1; CHDL; FLJ22530 | CHD1L | chromodomain helicase DNA binding |
| | | | protein 1-like |
| 0.587 | KIAA0355 | KIAA0355 | KIAA0355 |
| 0.557 | KIAA1615 | ZNF529 | zinc finger protein 529 |
| 0.554 | MGC2146 | IL11RA | interleukin 11 receptor, alpha |
| 0.552 | RNF84; MGC: 39780 | TRAF5 | TNF receptor-associated factor 5 |
| 0.551 | FLJ11795; MGC126013; | FLJ11795 | ankyrin repeat domain 55 |
| | MGC126014 | | |
| 0.548 | DKFZp686O1788 | MTX3 | metaxin 3 |
| 0.544 | DABP | DBP | D site of albumin promoter (albumin D-box) |
| | | | binding protein |
| 0.541 | FISH; SH3MD1 | SH3PXD2A | SH3 and PX domains 2A |
| 0.524 | CLAX; LLT1; OCIL | CLEC2D | C-type lectin domain family 2, member D |
| 0.518 | HPF1; FLJ11015; FLJ14876; | ZNF83 | zinc finger protein 83 |
| | FLJ90585; MGC33853 | | |
| 0.514 | ZCW4; ZCWCC2; FLJ11565; | MORC4 | MORC family CW-type zinc finger 4 |
| | dJ75H8.2 | | |
| 0.512 | RTS; TYMSAS; RTS beta; | ENOSF1 | enolase superfamily member 1 |
| | HSRTSBETA; RTS alpha | | |
| 0.483 | C7orf32; ATP6V0E2L | ATP6V0E2L | ATPase, H+ transporting V0 subunit e2 |
| 0.458 | PLC1; PLC-II; PLC148; | PLCG1 | phospholipase C, gamma 1 |
| | PLCgamma1 | | |
| 0.428 | RLK; TKL; BTKL; PTK4; | TXK | TXK tyrosine kinase |
| | PSCTK5; MGC22473 | | |
| 0.367 | T14; S152; Tp55; TNFRSF7; | TNFRSF7 | CD27 molecule |
| | MGC20393 | | |

TABLE 7P
M3.9 PTB v. Control, Genes Underrepresented in Active TB.

| Relative normalised | | | |
|---|---|---|---|
| expression | Common Name | Gene Symbol | Description |
| | P22_15_PTBvCSelect_09May08_PAL2Ttest_DOWN_M3.9 | | |
| 0.869 | ABC43; PMP70; PXMP1 | ABCD3 | ATP-binding cassette, sub-family D (ALD), |
| | | | member 3 |
| 0.86 | SPG8; MGC111053 | KIAA0196 | KIAA0196 |
| 0.859 | PUMH; HSPUM; PUMH1; | PUM1 | pumilio homolog 1 (Drosophila) |
| | PUML1; KIAA0099 | | |
| 0.856 | ASF; SF2; SF2p33; SRp30a; | SFRS1 | splicing factor, arginine/serine-rich 1 |
| | MGC5228 | | (splicing factor 2, alternate splicing factor) |
| 0.848 | DKFZp779N2044 | KIAA0528 | KIAA0528 |
| 0.843 | ALG6 | ALG6 | asparagine-linked glycosylation 6 homolog |
| | | | (S. cerevisiae, alpha-1,3- |
| | | | glucosyltransferase) |
| 0.829 | MGC111579; | DARS | aspartyl-tRNA synthetase |
| | DKFZp781B11202 | | |
| 0.829 | ADDL | ADD3 | adducin 3 (gamma) |
| 0.829 | KOX18; ZNF36; PHZ-37; | ZKSCAN1 | zinc finger with KRAB and SCAN |
| | ZNF139; MGC138429; | | domains 1 |
| | 9130423L19Rik | | |
| 0.826 | RPD3; YAF1 | HDAC2 | histone deacetylase 2 |
| 0.825 | FLJ21634; MGC71630 | GALNT11 | UDP-N-acetyl-alpha-D- |
| | | | galactosamine:polypeptide N- |
| | | | acetylgalactosaminyltransferase 11 |
| | | | (GalNAc-T11) |
| 0.816 | POLZ; REV3 | REV3L | REV3-like, catalytic subunit of DNA |
| | | | polymerase zeta (yeast) |
| 0.812 | Ki; PA28G; REG-GAMMA; | PSME3 | proteasome (prosome, macropain) activator |
| | PA28-gamma | | subunit 3 (PA28 gamma; Ki) |
| 0.811 | BRM; SNF2; SWI2; hBRM; | SMARCA2 | SWI/SNF related, matrix associated, actin |
| | Sth1p; BAF190; SNF2L2; | | dependent regulator of chromatin, subfamily |
| | SNF2LA; hSNF2a; FLJ36757; | | a, member 2 |
| | MGC74511 | | |
| 0.807 | ZNT5; ZTL1; ZNTL1; ZnT-5; | SLC30A5 | solute carrier family 30 (zinc transporter), |
| | MGC5499; FLJ12496; | | member 5 |
| | FLJ12756 | | |
| 0.802 | RAB7L; DKFZp686P1051 | RAB7L1 | RAB7, member RAS oncogene family-like 1 |
| 0.796 | ASCIZ; KIAA0431; | ASCIZ | ATM/ATR-Substrate Chk2-Interacting |
| | DKFZp779K1455 | | Zn2+-finger protein |
| 0.796 | TAF2B; CIF150; TAFII150 | TAF2 | TAF2 RNA polymerase II, TATA box |
| | | | binding protein (TBP)-associated factor, |
| | | | 150 kDa |
| 0.786 | N4WBP5; MGC10924 | NDFIP1 | Nedd4 family interacting protein 1 |
| 0.782 | PAP41; MGC117304; | PRPSAP2 | phosphoribosyl pyrophosphate synthetase- |
| | MGC126719; MGC126721 | | associated protein 2 |
| 0.779 | FLJ22584 | TTC13 | tetratricopeptide repeat domain 13 |
| 0.775 | CLCI; ICln; CLNS1B | CLNS1A | chloride channel, nucleotide-sensitive, 1A |
| 0.772 | LRRC5; FLJ10470; FLJ20403 | LRRC8D | leucine rich repeat containing 8 family, |
| | | | member D |
| 0.77 | CCT6; Cctz; HTR3; TCPZ; | CCT6A | chaperonin containing TCP1, subunit 6A |
| | TCP20; MoDP-2; TTCP20; | | (zeta 1) |
| | CCT-zeta; MGC126214; | | |
| | MGC126215; CCT-zeta-1; | | |
| | TCP-1-zeta | | |
| 0.765 | TOK-1 | BCCIP | BRCA2 and CDKN1A interacting protein |
| 0.764 | G3BP; HDH-VIII; | G3BP | GTPase activating protein (SH3 domain) |
| | MGC111040 | | binding protein 1 |
| 0.763 | FACT; CDC68; FACTP140; | SUPT16H | suppressor of Ty 16 homolog |
| | | | (S. cerevisiae) |
| | FLJ10857; FLJ14010; | | |
| | FLJ34357; SPT16/CDC68 | | |
| 0.757 | FBP2; FLJ12799; FLJ38170 | C14orf135 | chromosome 14 open reading frame 135 |
| 0.753 | GCP3; SPBC98; Spc98p | TUBGCP3 | tubulin, gamma complex associated protein 3 |
| 0.752 | FLJ13576; DKFZp564C012 | FLJ13576 | transmembrane protein 168 |
| 0.751 | SRP72 | SRP72 | signal recognition particle 72 kDa |
| 0.75 | CIA1; WDR39 | WDR39 | cytosolic iron-sulfur protein assembly 1 |
| | | | homolog (S. cerevisiae) |
| 0.738 | HPT; MRS2; MGC78523 | MRS2L | MRS2-like, magnesium homeostasis factor |
| | | | (S. cerevisiae) |
| 0.729 | CED-4; FLASH; RIP25; | CASP8AP2 | CASP8 associated protein 2 |
| | FLJ11208; KIAA1315 | | |
| 0.728 | PTPLB | PTPLB | protein tyrosine phosphatase-like (proline |
| | | | instead of catalytic arginine), member b |
| 0.724 | CHAC; FLJ42030; KIAA0986 | VPS13A | vacuolar protein sorting 13 homolog A |
| | | | (S. cerevisiae) |
| 0.724 | REC14 | WDR61 | WD repeat domain 61 |
| 0.719 | EB9; PDAF; RCAS1 | EBAG9 | estrogen receptor binding site associated, |
| | | | antigen, 9 |
| 0.712 | SNX4 | SNX4 | sorting nexin 4 |
| 0.704 | TOPIIB; top2beta | TOP2B | topoisomerase (DNA) II beta 180 kDa |
| 0.704 | CGI-12; FLJ10939 | MTERFD1 | MTERF domain containing 1 |
| 0.703 | CBC2; NIP1; CBP20; PIG55 | NCBP2 | nuclear cap binding protein subunit 2, |
| | | | 20 kDa |
| 0.702 | HAD; HHF4; HADH1; | HADHSC | hydroxyacyl-Coenzyme A dehydrogenase |
| | SCHAD; HADHSC; | | |
| | M/SCHAD; MGC8392 | | |
| 0.701 | p56; HSD8; FLJ11088; | DKFZP779L1558 | coiled-coil domain containing 91 |
| | DKFZP779L1558; | | |
| | DKFZp779L1558 | | |
| 0.701 | CREB; MGC9284 | CREB1 | cAMP responsive element binding protein 1 |
| 0.7 | AIP5; Tiul1; hSDRP1; | WWP1 | WW domain containing E3 ubiquitin protein |
| | DKFZp434D2111 | | ligase 1 |
| 0.681 | TAT-SF1; dJ196E23.2 | HTATSF1 | HIV-1 Tat specific factor 1 |
| 0.674 | LDLC | COG2 | component of oligomeric golgi complex 2 |
| 0.671 | HC71; CGI-150; C17orf25 | C17orf25 | glyoxalase domain containing 4 |
| 0.67 | GABAT; NPD009; GABA-AT | ABAT | 4-aminobutyrate aminotransferase |
| 0.668 | AKAP18 | AKAP7 | A kinase (PRKA) anchor protein 7 |
| 0.661 | LSFC; GP130; LRP130; | LRPPRC | leucine-rich PPR-motif containing |
| | CLONE-23970 | | |
| 0.644 | SCC-112; PIG54; FLJ41012; | SCC-112 | SCC-112 protein |
| | KIAA0648; MGC131948; | | |
| | MGC161503; | | |
| | DKFZp686B19246 | | |
| 0.643 | GDE | AGL | amylo-1,6-glucosidase, 4-alpha- |
| | | | glucanotransferase (glycogen debranching |
| | | | enzyme, glycogen storage disease type III) |
| 0.643 | NIP3 | BNIP3 | BCL2/adenovirus E1B 19 kDa interacting |
| | | | protein 3 |
| 0.64 | HSSB; RF-A; RP-A; REPA1; | RPA1 | replication protein A1, 70 kDa |
| | RPA70 | | |
| 0.63 | TAF2C; TAF4A; TAF2C1; | TAF4 | TAF4 RNA polymerase II, TATA box |
| | FLJ41943; TAFII130; | | binding protein (TBP)-associated factor, |
| | TAFII135 | | 135 kDa |
| 0.626 | TMP21; S31I125; Tmp-21-I; | TMED10 | transmembrane emp24-like trafficking |
| | S31III125; P24(DELTA) | | protein 10 (yeast) |
| 0.617 | FLJ20397; FLJ25564; | FLJ20397 | HEAT repeat containing 2 |
| | FLJ31671; FLJ39381 | | |
| 0.612 | CHA; Figlb; E2BP-1; | TCFL5 | transcription factor-like 5 (basic helix-loop- |
| | MGC46135 | | helix) |
| 0.588 | SRB; Cctd; MGC126164; | CCT4 | chaperonin containing TCP1, subunit 4 |
| | MGC126165 | | (delta) |
| 0.582 | Seh1; SEH1A; SEH1B; | SEH1L | SEH1-like (S. cerevisiae) |
| | SEC13L | | |
| 0.527 | HSU79274 | C12orf24 | chromosome 12 open reading frame 24 |

TABLE 8A
M1.5 LTB v. Control, Genes Underrepresented in Latent TB.

| Relative | | | |
|---|---|---|---|
| normalised | | Gene | |
| expression | Common Name | Symbol | Description |
| | P22_15_LTBvCSelect_09May08_PAL2Ttest_DOWN_M1.5 | | |
| 2.007 | STF1; STFA | CSTA | cystatin A (stefin A) |
| 1.915 | LSH; NRAMP; NRAMP1 | SLC11A1 | solute carrier family 11 (proton-coupled |
| | | | divalent metal ion transporters), member 1 |
| 1.903 | EZI; Zfp467 | ZNF467 | zinc finger protein 467 |
| 1.813 | TIL4; CD282 | TLR2 | toll-like receptor 2 |
| 1.811 | HSULF-2; FLJ90554; | SULF2 | sulfatase 2 |
| | KIAA1247; MGC126411; | | |
| | DKFZp313E091 | | |
| 1.716 | FLJ22662 | FLJ22662 | hypothetical protein FLJ22662 |
| 1.691 | FDF03 | PILRA | paired immunoglobin-like type 2 receptor |
| | | | alpha |
| 1.686 | HET; ITM; BWR1A; IMPT1; | SLC22A18 | solute carrier family 22 (organic cation |
| | TSSC5; ORCTL2; BWSCR1A; | | transporter), member 18 |
| | SLC22A1L; p45-BWR1A; | | |
| | DKFZp667A184 | | |
| 1.682 | ILT1; LIR7; CD85H; LIR-7 | LILRA2 | leukocyte immunoglobulin-like receptor, |
| | | | subfamily A (with TM domain), member 2 |
| 1.657 | C1QR1; C1qRP; CDw93; | C1QR1 | CD93 molecule |
| | MXRA4; C1qR(P); dJ737E23.1 | | |
| 1.636 | NCF; MGC3810; P40PHOX; | NCF4 | neutrophil cytosolic factor 4, 40 kDa |
| | SH3PXD4 | | |
| 1.623 | NOXA2; p67phox; P67-PHOX | NCF2 | neutrophil cytosolic factor 2 (65 kDa, |
| | | | chronic granulomatous disease, autosomal |
| | | | 2) |
| 1.542 | FLJ10357; SOLO | FLJ10357 | hypothetical protein FLJ10357 |
| 1.525 | JTK9 | HCK | hemopoietic cell kinase |
| 1.521 | FEM-2; POPX2; hFEM-2; | PPM1F | protein phosphatase 1F (PP2C domain |
| | CaMKPase; KIAA0015 | | containing) |
| 1.498 | CD32; FCG2; FcGR; CD32A; | FCGR2A | Fc fragment of IgG, low affinity IIa, |
| | CDw32; FCGR2; IGFR2; | | receptor (CD32) |
| | FCGR2A1; MGC23887; | | |
| | MGC30032 | | |
| 1.493 | DHRS8; PAN1B; RETSDR2; | DHRS8 | hydroxysteroid (17-beta) dehydrogenase 11 |
| | 17-BETA-HSD11; 17-BETA- | | |
| | HSDXI | | |
| 1.482 | FLJ11151; CSTP1 | FLJ11151 | hypothetical protein FLJ11151 |
| 1.478 | CD31; PECAM-1 | PECAM1 | platelet/endothelial cell adhesion molecule |
| | | | (CD31 antigen) |
| 1.469 | DORA | IGSF6 | immunoglobulin superfamily, member 6 |
| 1.452 | GP; G1RZFP; GOLIATH; | RNF130 | ring finger protein 130 |
| | MGC99542; MGC117241; | | |
| | MGC138647 | | |
| 1.45 | MLN70; S100C | S100A11 | S100 calcium binding protein A11 |
| 1.449 | MGC3886 | CTSS | cathepsin S |
| 1.425 | APPH; APPL2; CDEBP | APLP2 | amyloid beta (A4) precursor-like protein 2 |
| 1.41 | IMPD; RP10; IMPD1; LCA11; | IMPDH1 | IMP (inosine monophosphate) |
| | sWSS2608; DKFZp781N0678 | | dehydrogenase 1 |
| 1.406 | FCNM | FCN1 | ficolin (collagen/fibrinogen domain |
| | | | containing) 1 |
| 1.376 | MYD88 | MYD88 | myeloid differentiation primary response |
| | | | gene (88) |
| 1.371 | B144; LST-1; D6S49E; | LST1 | leukocyte specific transcript 1 |
| | MGC119006; MGC119007 | | |
| 1.348 | OS9 | OS9 | amplified in osteosarcoma |
| 1.334 | TEM7R; FLJ14623 | PLXDC2 | plexin domain containing 2 |
| 1.334 | Rab22B | RAB31 | RAB31, member RAS oncogene family |
| 1.301 | TS; TXS; CYP5; THAS; | TBXAS1 | thromboxane A synthase 1 (platelet, |
| | TXAS; CYP5A1 | | cytochrome P450, family 5, subfamily A) |
| 1.292 | HXK3; HKIII | HK3 | hexokinase 3 (white cell) |
| 1.292 | RISC; HSCP1 | SCPEP1 | serine carboxypeptidase 1 |
| 1.283 | IBA1; AIF-1; IRT-1 | AIF1 | allograft inflammatory factor 1 |
| 1.283 | CD14 | CD14 | CD14 molecule |
| 1.27 | PI; A1A; AAT; PI1; A1AT; | SERPINA1 | serpin peptidase inhibitor, clade A (alpha-1 |
| | MGC9222; PRO2275; | | antiproteinase, antitrypsin), member 1 |
| | MGC23330 | | |
| 1.261 | LIR6; CD85I; LIR-6; | LILRA1 | leukocyte immunoglobulin-like receptor, |
| | MGC126563 | | subfamily A (with TM domain), member 1 |
| 1.221 | CAP102; FLJ36832 | CTNNA1 | catenin (cadherin-associated protein), alpha |
| | | | 1, 102 kDa |
| 1.192 | BCKDK | BCKDK | branched chain ketoacid dehydrogenase |
| | | | kinase |
| 1.137 | p75; TBPII; TNFBR; TNFR2; | TNFRSF1B | tumor necrosis factor receptor superfamily, |
| | CD120b; TNFR80; TNF-R75; | | member 1B |
| | p75TNFR; TNF-R-II | | |

TABLE 8B
M2.1 LTB v. Control, Genes Overrepresented in Latent TB.

| Relative | | | |
|---|---|---|---|
| normalised | | | |
| expression | Common Name | Gene Symbol | Description |
| | P22_15_LTBvCSelect_09May08_PAL2Ttest_UP_M2.01 | | |
| 0.801 | LIME; LP8067; FLJ20406; | LIME1 | Lck interacting transmembrane adaptor 1 |
| | dJ583P15.4; RP4-583P15.5 | | |
| 0.769 | FLJ34563; MGC35163 | SAMD3 | sterile alpha motif domain containing 3 |
| 0.763 | SISd; SCYA5; RANTES; | CCL5 | chemokine (C-C motif) ligand 5 |
| | TCP228; D17S136E; | | |
| | MGC17164 | | |
| 0.758 | ORP7; MGC71150 | OSBPL7 | oxysterol binding protein-like 7 |
| 0.757 | | LOC387882 | |
| 0.736 | SLP2; SGA72M; CHR11SYT; | SYTL2 | synaptotagmin-like 2 |
| | KIAA1597; MGC102768 | | |
| 0.735 | DORZ1; DKFZP564O243 | ABHD14A | abhydrolase domain containing 14A |
| 0.727 | MGC33870; MGC74858 | NCALD | neurocalcin delta |
| 0.691 | LPAP; CD45-AP; | PTPRCAP | protein tyrosine phosphatase, receptor type, |
| | MGC138602; MGC138603 | | C-associated protein |
| 0.686 | T11; SRBC | CD2 | CD2 molecule |
| 0.671 | CD8; MAL; p32; Leu2 | CD8A | CD8a molecule |
| 0.656 | HOP; OB1; LAGY; Toto; | HOP | homeodomain-only protein |
| | Cameo; NECC1; SMAP31; | | |
| | MGC20820 | | |
| 0.651 | 2F1; MAFA; MAFA-L; | KLRG1 | killer cell lectin-like receptor subfamily G, |
| | CLEC15A; MAFA-2F1; | | member 1 |
| | MGC13600 | | |
| 0.65 | | LOC197135 | |
| 0.643 | GIG1 | NKG7 | natural killer cell group 7 sequence |
| 0.638 | TSAd; F2771 | SH2D2A | SH2 domain protein 2A |
| 0.634 | FEOM; CFEOM; FEOM1; | KIF21A | kinesin family member 21A |
| | CFEOM1; FLJ20052; | | |
| | KIAA1708; DKFZp779C159 | | |
| 0.627 | KIAA0442; MGC13140 | AUTS2 | autism susceptibility candidate 2 |
| 0.583 | BFPP; TM7LN4; TM7XN1; | GPR56 | G protein-coupled receptor 56 |
| | DKFZp781L1398 | | |
| 0.572 | TARP; CD3G; TCRG; | TARP | TCR gamma alternate reading frame protein |
| | TCRGC1; TCRGC2 | | |
| 0.502 | 519; LAG2; NKG5; LAG-2; | GNLY | granulysin |
| | D2S69E; TLA519 | | |
| 0.303 | CCP-X; CGL-2; CSP-C; | GZMH | granzyme H (cathepsin G-like 2, protein h- |
| | CTLA1; CTSGL2 | | CCPX) |

TABLE 8C
M2.6 LTB v. Control, Genes Underrepresented in Latent TB.

| Relative | | | |
|---|---|---|---|
| normalised | | | |
| expression | Common Name | Gene Symbol | Description |
| | P22_15_LTBvCSelect_09May08_PAL2Ttest_DOWN_M2.06 | | |
| | Module 2.06, myeloid, fold | | |
| | change is healthy relative to | | |
| | LTB, ie DOWN in LTB | | |
| 2.409 | HsT287 | ZNF516 | zinc finger protein 516 |
| 2.286 | CRISP11; LCRISP2; | CRISPLD2 | cysteine-rich secretory protein LCCL |
| | MGC74865; DKFZP434B044 | | domain containing 2 |
| 2.177 | MAG1; GPAT3; AGPAT8; | HMFN0839 | lung cancer metastasis-associated protein |
| | MGC11324 | | |
| 2.095 | CDD | CDA | cytidine deaminase |
| 2.094 | CRBP4; CRBPIV; MGC70641 | RBP7 | retinol binding protein 7, cellular |
| 1.917 | SSC1; HsT17287 | AQP9 | aquaporin 9 |
| 1.916 | GMR; CD116; CSF2R; | CSF2RA | colony stimulating factor 2 receptor, alpha, |
| | CDw116; CSF2RX; CSF2RY; | | low-affinity (granulocyte-macrophage) |
| | GMCSFR; CSF2RAX; | | |
| | CSF2RAY; MGC3848; | | |
| | MGC4838; GM-CSF-R-alpha | | |
| 1.853 | G0S8 | RGS2 | regulator of G-protein signalling 2, 24 kDa |
| 1.734 | HKII; HXK2; | HK2 | hexokinase 2 |
| | DKFZp686M1669 | | |
| 1.734 | BB1 | LENG4 | leukocyte receptor cluster (LRC) member 4 |
| 1.701 | UB1; CEP3; BORG2; | CDC42EP3 | CDC42 effector protein (Rho GTPase |
| | FLJ46903 | | binding) 3 |
| 1.671 | SPAL2; FLJ23126; FLJ23632; | SIPA1L2 | signal-induced proliferation-associated 1 |
| | KIAA1389 | | like 2 |
| 1.669 | ST1; SYCL; MDA-9; TACIP18 | SDCBP | syndecan binding protein (syntenin) |
| 1.669 | CAN; CAIN; N214; D9S46E; | NUP214 | nucleoporin 214 kDa |
| | MGC104525 | | |
| 1.651 | | SLC19A1 | |
| 1.65 | LPB3; S1P3; EDG-3; S1PR3; | EDG3 | endothelial differentiation, sphingolipid G- |
| | FLJ37523; MGC71696 | | protein-coupled receptor, 3 |
| 1.642 | FPR; FMLP | FPR1 | formyl peptide receptor 1 |
| 1.61 | GPCR1; GPR86; GPR94; | P2RY13 | purinergic receptor P2Y, G-protein coupled, |
| | P2Y13; SP174; FKSG77 | | 13 |
| 1.606 | WDR80; FLJ00012 | ATG16L2 | ATG16 autophagy related 16-like 2 (S. cerevisiae) |
| 1.601 | LENG5; SEN34; SEN34L | TSEN34 | tRNA splicing endonuclease 34 homolog |
| | | | (S. cerevisiae) |
| 1.575 | FPF; p55; p60; TBP1; TNF-R; | TNFRSF1A | tumor necrosis factor receptor superfamily, |
| | TNFAR; TNFR1; p55-R; | | member 1A |
| | CD120a; TNFR55; TNFR60; | | |
| | TNF-R-I; TNF-R55; | | |
| | MGC19588 | | |
| 1.572 | PELI2 | PELI2 | pellino homolog 2 (Drosophila) |
| 1.562 | FLJ13052; FLJ37724; | NADK | NAD kinase |
| | dJ283E3.1; RP1-283E3.6 | | |
| 1.558 | 5-LO; 5LPG; LOG5; | ALOX5 | arachidonate 5-lipoxygenase |
| | MGC163204 | | |
| 1.534 | TMPIT | TMPIT | transmembrane protein induced by tumor |
| | | | necrosis factor alpha |
| 1.517 | FLJ31978 | GLT1D1 | glycosyltransferase 1 domain containing 1 |
| 1.517 | PFKFB4 | PFKFB4 | 6-phosphofructo-2-kinase/fructose-2,6- |
| | | | biphosphatase 4 |
| 1.516 | FLJ22470; KIAA1993; | ZBTB34 | zinc finger and BTB domain containing 34 |
| | MGC24652; RP11-106H5.1 | | |
| 1.482 | P39; VATX; VMA6; ATP6D; | ATP6V0D1 | ATPase, H+ transporting, lysosomal 38 kDa, |
| | ATP6DV; VPATPD | | V0 subunit d1 |
| 1.473 | PRAM-1; MGC39864 | PRAM1 | PML-RARA regulated adaptor molecule 1 |
| 1.471 | BIT; MFR; P84; SIRP; MYD- | PTPNS1 | signal-regulatory protein alpha |
| | 1; SHPS1; CD172A; PTPNS1; | | |
| | SHPS-1; SIRPalpha; | | |
| | SIRPalpha2; SIRP-ALPHA-1 | | |
| 1.463 | M130; MM130 | CD163 | CD163 molecule |
| 1.434 | AF-1; IFGR2; IFNGT1 | IFNGR2 | interferon gamma receptor 2 (interferon |
| | | | gamma transducer 1) |
| 1.405 | RALB | RALB | v-ral simian leukemia viral oncogene |
| | | | homolog B (ras related; GTP binding |
| | | | protein) |
| 1.405 | SLCO3A1 | SLCO3A1 | solute carrier organic anion transporter |
| | | | family, member 3A1; synonyms: OATP-D, |
| | | | OATP3A1, FLJ40478, SLC21A11; solute |
| | | | carrier family 21 (organic anion |
| | | | transporter), member 11; Homo sapiens |
| | | | solute carrier organic anion transporter |
| | | | family, member 3A1 (SLCO3A1), mRNA. |
| 1.397 | PTPE; HPTPE; | PTPRE | protein tyrosine phosphatase, receptor type, E |
| | DKFZp313F1310; R-PTP- | | |
| | EPSILON | | |
| 1.397 | RCC4; FLJ14784 | DIRC2 | disrupted in renal carcinoma 2 |
| 1.396 | DAP12; KARAP; PLOSL | TYROBP | TYRO protein tyrosine kinase binding |
| | | | protein |
| 1.371 | B144; LST-1; D6S49E; | LST1 | leukocyte specific transcript 1 |
| | MGC119006; MGC119007 | | |
| 1.359 | BFD; PFC; PFD; PROPERDIN | PFC | complement factor properdin |
| 1.31 | CAG4A; ERDA5; PRAT4A | TNRC5 | trinucleotide repeat containing 5 |
| 1.307 | CD18; TNFCR; D12S370; | LTBR | lymphotoxin beta receptor (TNFR |
| | TNFR-RP; TNFRSF3; TNFR2- | | superfamily, member 3) |
| | RP; LT-BETA-R; TNF-R-III | | |
| 1.305 | CEB | VAMP3 | vesicle-associated membrane protein 3 |
| | | | (cellubrevin) |
| 1.304 | CSC-21K | TIMP2 | TIMP metallopeptidase inhibitor 2 |
| 1.301 | BPOZ; EF1ABP; PP2259; | ABTB1 | ankyrin repeat and BTB (POZ) domain |
| | MGC20585 | | containing 1 |
| 1.294 | C6orf209; FLJ11240; | LMBRD1 | LMBR1 domain containing 1 |
| | bA810I22.1; RP11-810I22.1 | | |
| 1.266 | PBF; C21orf1; C21orf3 | PTTG1IP | pituitary tumor-transforming 1 interacting |
| | | | protein |
| 1.235 | ZFYVE10; FLJ32333; | MTMR3 | myotubularin related protein 3 |
| | KIAA0371; FYVE-DSP1 | | |
| 1.216 | CFP1; CBCP1; C10orf9 | C10orf9 | cyclin Y |
| 1.2 | SPT4H; SUPT4H | SUPT4H1 | suppressor of Ty 4 homolog 1 (S. cerevisiae) |

TABLE 8D
M2.10 LTB v. Control, Genes Underrepresented in Latent TB.

| Relative | | | |
|---|---|---|---|
| normalised | | | |
| expression | Common Name | Gene Symbol | Description |
| | P22_15_LTBvCSelect_09May08_PAL2Ttest_DOWN_M2.10 | | |
| | Undefined module M2.10, fold | | |
| | change healthy relative to LTB, | | |
| | ie DOWN in LTB | | |
| 1.608 | JAML; AMICA; Gm638; | AMICA1 | adhesion molecule, interacts with |
| | CREA7-1; CREA7-4; | | CXADR antigen 1 |
| | FLJ37080; MGC118814; | | |
| | MGC118815 | | |
| 1.537 | MPEG1; MGC132657; | MPEG1 | macrophage expressed gene 1 |
| | MGC138435 | | |
| 1.514 | L13; MGC13061 | RNF135 | ring finger protein 135 |
| 1.507 | PAKalpha; MGC130000; | PAK1 | p21/Cdc42/Rac1-activated kinase 1 |
| | MGC130001 | | (STE20 homolog, yeast) |
| 1.471 | T49; pT49 | FGL2 | fibrinogen-like 2 |
| 1.405 | KIAA0513 | KIAA0513 | KIAA0513 |
| 1.396 | NCKX4; SLC24A2; FLJ38852 | SLC24A4 | solute carrier family 24 |
| | | | (sodium/potassium/calcium exchanger), |
| | | | member 4 |
| 1.358 | FLJ34389 | MLKL | mixed lineage kinase domain-like |
| 1.348 | ETO2; MTG16; MTGR2; | CBFA2T3 | core-binding factor, runt domain, alpha |
| | ZMYND4 | | subunit 2; translocated to, 3 |
| 1.331 | IRC1; IRC2; IRp60; IGSF12; | CD300A | CD300a molecule |
| | CMRF35H; CMRF-35H; | | |
| | CMRF35H9; CMRF-35-H9 | | |
| 1.3 | GLIPR; RTVP1; CRISP7 | GLIPR1 | GLI pathogenesis-related 1 (glioma) |
| 1.229 | ENC-1AS | HEXB | hexosaminidase B (beta polypeptide) |
| 1.222 | TIRP; TRAM; TIRAP3; | TICAM2 | toll-like receptor adaptor molecule 2 |
| | TICAM-2; MGC129876; | | |
| | MGC129877 | | |
| 1.175 | FLJ31265 | NUDT16 | nudix (nucleoside diphosphate linked |
| | | | moiety X)-type motif 16 |
| 1.17 | FKBP133; KIAA0674 | KIAA0674 | FK506 binding protein 15, 133 kDa |

TABLE 8E
M3.2 LTB v. Control, Genes Underrepresented in Latent TB.

| Relative | | | |
|---|---|---|---|
| normalised | | | |
| expression | Common Name | Gene Symbol | Description |
| | P22_15_LTBvCSelect_09May08_PAL2Ttest_DOWN_M3.2 | | |
| | Inflammation 3.2 fold change is | | |
| | healthy relative to LTB, ie | | |
| | DOWN in LTB | | |
| 4.289 | K60; NAF; GCP1; LECT; | IL8 | interleukin 8 |
| | LUCT; NAP1; 3-10C; CXCL8; | | |
| | GCP-1; LYNAP; MDNCF; | | |
| | MONAP; NAP-1; SCYB8; | | |
| | TSG-1; AMCF-I; b-ENAP | | |
| 2.068 | CD87; UPAR; URKR | PLAUR | plasminogen activator, urokinase receptor |
| 2.009 | PBEF; NAMPT; MGC117256; | PBEF1 | pre-B-cell colony enhancing factor 1 |
| | DKFZP666B131; | | |
| | 1110035O14Rik | | |
| 1.9 | | IER3 | |
| 1.87 | TREM-1 | TREM1 | triggering receptor expressed on myeloid |
| | | | cells 1 |
| 1.79 | E4BP4; IL3BP1; NFIL3A; NF- | NFIL3 | nuclear factor, interleukin 3 regulated |
| | IL3A | | |
| 1.739 | KIAA1145 | TMCC3 | transmembrane and coiled-coil domain |
| | | | family 3 |
| 1.728 | PINH; FLJ21759; FLJ23500; | TP53INP2 | tumor protein p53 inducible nuclear |
| | C20orf110; dJ1181N3.1; | | protein 2 |
| | DKFZp434B2411; | | |
| | DKFZp434O0827 | | |
| 1.705 | MAD; MAD1; MGC104659 | MXD1 | MAX dimerization protein 1 |
| 1.657 | SGK1 | SGK | serum/glucocorticoid regulated kinase |
| 1.654 | SLCO3A1 | SLCO3A1 | solute carrier organic anion transporter |
| | | | family, member 3A1; synonyms: |
| | | | OATP-D, OATP3A1, FLJ40478, |
| | | | SLC21A11; solute carrier family 21 |
| | | | (organic anion transporter), member 11; |
| | | | Homo sapiens solute carrier |
| | | | organic anion transporter family, |
| | | | member 3A1 (SLCO3A1), mRNA. |
| 1.637 | C5orf6 | FAM53C | family with sequence similarity 53, |
| | | | member C |
| 1.632 | PDLIM7 | PDLIM7 | PDZ and LIM domain 7 (enigma) |
| 1.591 | NIN1; NINJURIN | NINJ1 | ninjurin 1 |
| 1.572 | RIT; RIBB; ROC1; | RIT1 | Ras-like without CAAX 1 |
| | MGC125864; MGC125865 | | |
| 1.567 | SB135 | MYADM | myeloid-associated differentiation marker |
| 1.54 | RCP; NOEL1A; FLJ22524; | RAB11FIP1 | RAB11 family interacting protein 1 |
| | FLJ22622; MGC78448; rab11- | | (class I) |
| | FIP1; DKFZp686E2214 | | |
| 1.526 | DANGER; bA127L20; | KIAA1754 | KIAA1754 |
| | bA127L20.2; RP11-127L20.4 | | |
| 1.515 | | SPAG9 | |
| 1.499 | HSS; JLP; HLC4; PHET; | SPAG9 | sperm associated antigen 9 |
| | PIG6; FLJ13450; FLJ14006; | | |
| | FLJ26141; FLJ34602; | | |
| | KIAA0516; MGC14967; | | |
| | MGC74461; MGC117291 | | |
| 1.496 | MGC20461 | OSM | oncostatin M |
| 1.444 | KIAA1673 | CPEB4 | cytoplasmic polyadenylation element |
| | | | binding protein 4 |
| 1.433 | IL-1; IL1F2; IL1-BETA | IL1B | interleukin 1, beta |
| 1.413 | TRIP8; FLJ14374; KIAA1380; | JMJD1C | jumonji domain containing 1C |
| | RP11-10C13.2; | | |
| | DKFZp761F0118 | | |
| 1.41 | FLJ11080; FLJ33961; | FAM49A | family with sequence similarity 49, |
| | DKFZP566A1524 | | member A |
| 1.4 | EOPA; NUDEL; MITAP1; | NDEL1 | nudE nuclear distribution gene E homolog |
| | DKFZp451M0318 | | (A. nidulans)-like 1 |
| 1.384 | NHE8; FLJ42500; KIAA0939; | SLC9A8 | solute carrier family 9 (sodium/hydrogen |
| | MGC138418; | | exchanger), member 8 |
| | DKFZp686C03237 | | |
| 1.379 | FLJ14744 | PPP1R15B | protein phosphatase 1, regulatory |
| | | | (inhibitor) subunit 15B |
| 1.356 | PPG; PRG; PRG1; MGC9289; | PRG1 | serglycin |
| | FLJ12930 | | |
| 1.348 | ATG8; GEC1; APG8L | GABARAPL1 | GABA(A) receptor-associated protein |
| | | | like 1 |
| 1.332 | TTP; G0S24; GOS24; TIS11; | ZFP36 | zinc finger protein 36, C3H type, homolog |
| | NUP475; RNF162A | | (mouse) |
| 1.329 | PFK2; IPFK2 | PFKFB3 | 6-phosphofructo-2-kinase/fructose-2,6- |
| | | | biphosphatase 3 |
| 1.31 | DKFZp547M072 | MIDN | midnolin |
| 1.301 | FLJ13448 | COQ10B | coenzyme Q10 homolog B (S. cerevisiae) |
| 1.285 | C8FW; GIG2; SKIP1 | TRIB1 | tribbles homolog 1 (Drosophila) |
| 1.284 | FLJ13725; KIAA1930 | FAM65A | family with sequence similarity 65, |
| | | | member A |
| 1.272 | FLJ46337; MGC117209; | C15orf39 | chromosome 15 open reading frame 39 |
| | DKFZP434H132 | | |
| 1.258 | AII; AVP; FCU; MWS; FCAS; | CIAS1 | NLR family, pyrin domain containing 3 |
| | CIAS1; NALP3; C1orf7; | | |
| | CLR1.1; PYPAF1; AII/AVP; | | |
| | AGTAVPRL | | |
| 1.252 | BRF1; ERF1; cMG1; ERF-1; | ZFP36L1 | zinc finger protein 36, C3H type-like 1 |
| | Berg36; TIS11B; RNF162B | | |
| 1.249 | FRA2; FLJ23306 | FOSL2 | FOS-like antigen 2 |
| 1.235 | GADD34 | PPP1R15A | protein phosphatase 1, regulatory |
| | | | (inhibitor) subunit 15A |
| 1.235 | p33; p47; p33ING1; p24ING1c; | ING1 | inhibitor of growth family, member 1 |
| | p33ING1b; p47ING1a | | |
| 1.231 | P47; FLJ27168 | PLEK | pleckstrin |
| 1.218 | UBP; SIH003; MGC129878; | USP3 | ubiquitin specific peptidase 3 |
| | MGC129879 | | |
| 1.208 | Sei-2; TRIP-Br2; MGC126688; | SERTAD2 | SERTA domain containing 2 |
| | MGC126690 | | |
| 1.204 | DCTN4 | DCTN4 | dynactin 4 (p62) |
| 1.192 | ROX; MAD6; MXD6 | MNT | MAX binding protein |
| 1.165 | RBT1 | SERTAD3 | SERTA domain containing 3 |
| 1.157 | WIPI3; WIPI-3 | WDR45L | WDR45-like |
| 1.156 | ERF; RF1; ERF1; TB3-1; | ETF1 | eukaryotic translation termination factor 1 |
| | D5S1995; SUP45L1; | | |
| | MGC111066 | | |
| 1.156 | KIAA0118 | RAB21 | RAB21, member RAS oncogene family |
| 1.098 | MAPKAPK2 | MAPKAPK2 | mitogen-activated protein kinase-activated |
| | | | protein kinase 2 |

TABLE 8F
M3.3 LTB v. Control, Genes Underrepresented in Latent TB.

| Relative | | | |
|---|---|---|---|
| normalised | | Gene | |
| expression | Common Name | Symbol | Description |
| | P22_15_LTBvCSelect_09May08_PAL2Ttest_DOWN_M3.3 | | |
| | Inflammation 3.2 fold change is | | |
| | healthy relative to LTB, ie | | |
| | DOWN in LTB | | |
| 2.716 | QC; GCT | QPCT | glutaminyl-peptide cyclotransferase |
| | | | (glutaminyl cyclase) |
| 2.579 | CRE-BPA | CREB5 | cAMP responsive element binding protein 5 |
| 2.468 | APN; CD13; LAP1; PEPN; | ANPEP | alanyl (membrane) aminopeptidase |
| | gp150 | | (aminopeptidase N, aminopeptidase M, |
| | | | microsomal aminopeptidase, CD13, p150) |
| 2.426 | PAD; PDI4; PDI5; PADI5 | PADI4 | peptidyl arginine deiminase, type IV |
| 2.245 | MRP; WLS; C1orf139; | GPR177 | G protein-coupled receptor 177 |
| | FLJ23091; MGC14878; | | |
| | MGC131760 | | |
| 2 | HIS; HSTD; histidase | HAL | histidine ammonia-lyase |
| 1.963 | PYGL | PYGL | phosphorylase, glycogen; liver (Hers |
| | | | disease, glycogen storage disease type VI) |
| 1.948 | | EGFL5 | |
| 1.935 | L-H2; ASGP-R; CLEC4H2; | ASGR2 | asialoglycoprotein receptor 2 |
| | Hs.1259 | | |
| 1.892 | CD114; GCSFR | CSF3R | colony stimulating factor 3 receptor |
| | | | (granulocyte) |
| 1.882 | LAMPB; CD107b; LAMP-2C | LAMP2 | lysosomal-associated membrane protein 2 |
| 1.813 | ALFY; ZFYVE25; KIAA0993; | WDFY3 | WD repeat and FYVE domain containing 3 |
| | MGC16461 | | |
| 1.8 | STX3A | STX3A | syntaxin 3 |
| 1.771 | CR1 | CR1 | complement component (3b/4b) receptor 1 |
| | | | (Knops blood group); synonyms: KN, |
| | | | C3BR, CD35; isoform F precursor is |
| | | | encoded by transcript variant F; C3-binding |
| | | | protein; CD35 antigen; complement |
| | | | component receptor 1; C3b/C4b receptor; |
| | | | Knops blood group antigen; Homo sapiens |
| | | | complement component (3b/4b) receptor 1 |
| | | | (Knops blood group) (CR1), transcript |
| | | | variant F, mRNA. |
| 1.764 | DCL-1; BIMLEC; CLEC13A; | CD302 | CD302 molecule |
| | KIAA0022 | | |
| 1.758 | FER1L1; LGMD2B; | DYSF | dysferlin, limb girdle muscular dystrophy |
| | FLJ00175; FLJ90168 | | 2B (autosomal recessive) |
| 1.733 | TM6SF1 | TM6SF1 | transmembrane 6 superfamily member 1 |
| 1.721 | MYO1F | MYO1F | myosin IF |
| 1.691 | CPR8; KIAA1254 | CCPG1 | cell cycle progression 1 |
| 1.688 | LAB; NTAL; WSCR5; | LAT2 | linker for activation of T cells family, |
| | WBSCR5; HSPC046; | | member 2 |
| | WBSCR15 | | |
| 1.687 | CNAIP; FLJ40652; bK126B4.4 | NFAM1 | NFAT activating protein with ITAM motif 1 |
| 1.659 | FVL; PCCF; factor V | F5 | coagulation factor V (proaccelerin, labile |
| | | | factor) |
| 1.655 | FLJ20273; DKFZp686F02235 | FLJ20273 | RNA-binding protein |
| 1.647 | NR4; CD213A1; IL-13Ra | IL13RA1 | interleukin 13 receptor, alpha 1 |
| 1.636 | NCF; MGC3810; P40PHOX; | NCF4 | neutrophil cytosolic factor 4, 40 kDa |
| | SH3PXD4 | | |
| 1.635 | p63; CLIMP-63; ERGIC-63; | CKAP4 | cytoskeleton-associated protein 4 |
| | MGC99554 | | |
| 1.611 | SELR; SELX; MSRB1; | SEPX1 | selenoprotein X, 1 |
| | HSPC270; MGC3344 | | |
| 1.6 | MD-2 | LY96 | lymphocyte antigen 96 |
| 1.599 | NPL1; c112; C1orf13; | NPL | N-acetylneuraminate pyruvate lyase |
| | MGC61869; MGC149582 | | (dihydrodipicolinate synthase) |
| 1.59 | HAP; ASYIP; NSPL2; NSPLII; | RTN3 | reticulon 3 |
| | RTN3-A1 | | |
| 1.581 | VMP1; DKFZP566I133 | TMEM49 | transmembrane protein 49 |
| 1.567 | HBP; HEBP | HEBP1 | heme binding protein 1 |
| 1.562 | LAMPB; CD107b; LAMP-2C | LAMP2 | lysosomal-associated membrane protein 2 |
| 1.559 | C32; CKLF1; CKLF2; CKLF3; | CKLF | chemokine-like factor |
| | CKLF4; UCK-1; HSPC224 | | |
| 1.538 | | RASSF2 | |
| 1.532 | SemE; SEMAE | SEMA3C | sema domain, immunoglobulin domain (Ig), |
| | | | short basic domain, secreted, (semaphorin) |
| | | | 3C |
| 1.53 | ARAP3; DRAG1; FLJ21065 | CENTD3 | centaurin, delta 3 |
| 1.516 | HIG-1; C14orf75; FLJ36164; | TDRD9 | tudor domain containing 9 |
| | MGC135025; | | |
| | DKFZp434N0820 | | |
| 1.51 | CAMKK; CAMKKB; | CAMKK2 | calcium/calmodulin-dependent protein |
| | KIAA0787; MGC15254 | | kinase kinase 2, beta |
| 1.503 | MEKK3; MAPKKK3 | MAP3K3 | mitogen-activated protein kinase kinase |
| | | | kinase 3 |
| 1.488 | AC; PHP; ASAH; PHP32; | ASAH1 | N-acylsphingosine amidohydrolase (acid |
| | FLJ21558; FLJ22079 | | ceramidase) 1 |
| 1.484 | FCRN; alpha-chain | FCGRT | Fc fragment of IgG, receptor, transporter, |
| | | | alpha |
| 1.479 | MGC33054 | SNX10 | sorting nexin 10 |
| 1.474 | HO68; VA68; VPP2; Vma1; | ATP6V1A | ATPase, H+ transporting, lysosomal 70 kDa, |
| | ATP6A1; ATP6V1A1 | | V1 subunit A |
| 1.466 | MGST; GST12; MGST-I; | MGST1 | microsomal glutathione S-transferase 1 |
| | MGC14525 | | |
| 1.466 | GAIP; RGSGAIP | RGS19 | regulator of G-protein signalling 19 |
| 1.461 | TKT1; FLJ34765 | TKT | transketolase (Wernicke-Korsakoff |
| | | | syndrome) |
| 1.449 | S171 | NUMB | numb homolog (Drosophila) |
| 1.448 | FCHO2 | FCHO2 | FCH domain only 2 |
| 1.444 | LOC339745 | LOC339745 | hypothetical protein LOC339745 |
| 1.443 | CR3A; MO1A; CD11B; MAC- | ITGAM | integrin, alpha M (complement component 3 |
| | 1; MAC1A; MGC117044 | | receptor 3 subunit) |
| 1.442 | D54; hD54; DKFZp686A1765 | TPD52L2 | tumor protein D52-like 2 |
| 1.432 | MY014; KIAA0488; | SNX27 | sorting nexin family member 27 |
| | MGC20471; MGC126871; | | |
| | MGC126873 | | |
| 1.429 | QK; Hqk; QK3; | QKI | quaking homolog, KH domain RNA binding |
| | DKFZp586I0923 | | (mouse) |
| 1.424 | EVDB; D17S376 | EVI2B | ecotropic viral integration site 2B |
| 1.424 | PPT; CLN1; INCL | PPT1 | palmitoyl-protein thioesterase 1 (ceroid- |
| | | | lipofuscinosis, neuronal 1, infantile) |
| 1.405 | AOAH | AOAH | acyloxyacyl hydrolase (neutrophil) |
| 1.404 | MAY1; MGC49908; nPKC- | PRKCD | protein kinase C, delta |
| | delta | | |
| 1.39 | IMPA2 | IMPA2 | inositol(myo)-1(or 4)-monophosphatase 2 |
| 1.382 | ZYG11; FLJ13456 | ZYG11B | zyg-11 homolog B (C. elegans) |
| 1.366 | a3; Stv1; Vph1; Atp6i; OC116; | TCIRG1 | T-cell, immune regulator 1, ATPase, H+ |
| | OPTB1; TIRC7; ATP6N1C; | | transporting, lysosomal V0 subunit A3 |
| | ATP6V0A3; OC-116 kDa | | |
| 1.364 | PGCP | PGCP | plasma glutamate carboxypeptidase |
| 1.362 | NNA1; KIAA1035; | AGTPBP1 | ATP/GTP binding protein 1 |
| | DKFZp686M20191 | | |
| 1.355 | TTG2; RBTN2; RHOM2; | LMO2 | LIM domain only 2 (rhombotin-like 1) |
| | RBTNL1 | | |
| 1.344 | CIP1; FLJ46905 | SLC12A9 | solute carrier family 12 (potassium/chloride |
| | | | transporters), member 9 |
| 1.34 | ASRT5; IRAKM; IRAK-M | IRAK3 | interleukin-1 receptor-associated kinase 3 |
| 1.34 | NEU; SIAL1 | NEU1 | sialidase 1 (lysosomal sialidase) |
| 1.332 | CRFB4; CRF2-4; D21S58; | IL10RB | interleukin 10 receptor, beta |
| | D21S66; CDW210B; IL-10R2 | | |
| 1.321 | ASC; TMS1; CARD5; | PYCARD | PYD and CARD domain containing |
| | MGC10332 | | |
| 1.31 | KLHDC7C; KIAA0711 | KBTBD11 | kelch repeat and BTB (POZ) domain |
| | | | containing 11 |
| 1.308 | LTA4H | LTA4H | leukotriene A4 hydrolase |
| 1.307 | NR2B1; FLJ16020; FLJ16733; | RXRA | retinoid X receptor, alpha |
| | MGC102720 | | |
| 1.303 | JAM; KAT; JAM1; JAMA; | F11R | F11 receptor |
| | JCAM; CD321; JAM-1; JAM- | | |
| | A; PAM-1 | | |
| 1.298 | LH; LLH; PLOD | PLOD1 | procollagen-lysine 1,2-oxoglutarate 5- |
| | | | dioxygenase 1 |
| 1.285 | JTK8; FLJ26625 | LYN | v-yes-1 Yamaguchi sarcoma viral related |
| | | | oncogene homolog |
| 1.281 | MTX; MTXN | MTX1 | metaxin 1 |
| 1.28 | CGI-44 | SQRDL | sulfide quinone reductase-like (yeast) |
| 1.267 | FLJ20424 | C14orf94 | chromosome 14 open reading frame 94 |
| 1.248 | DCIR; LLIR; DDB27; | CLEC4A | C-type lectin domain family 4, member A |
| | CLECSF6; HDCGC13P | | |
| 1.238 | EI; LEI; PI2; MNEI; M/NEI; | SERPINB1 | serpin peptidase inhibitor, clade B |
| | ELANH2 | | (ovalbumin), member 1 |
| 1.234 | 3PK; MAPKAP3 | MAPKAPK3 | mitogen-activated protein kinase-activated |
| | | | protein kinase 3 |
| 1.227 | | ACSS2 | |
| 1.217 | H2A.y; H2A/y; H2AFJ; | H2AFY | H2A histone family, member Y |
| | mH2A1; H2AF12M; | | |
| | MACROH2A1.1; | | |
| | macroH2A1.2 | | |
| 1.213 | PP3856 | NAPRT1 | nicotinate phosphoribosyltransferase |
| | | | domain containing 1 |
| 1.212 | ESP-2; HED-2 | ZYX | zyxin |
| 1.179 | SPC18; SPCS4A; SEC11L1; | SEC11L1 | SEC11 homolog A (S. cerevisiae) |
| | sid2895; 1810012E07Rik | | |
| 1.173 | hEDTP; C3orf29; FLJ22405; | C3orf29 | myotubularin related protein 14 |
| | FLJ90311 | | |
| 1.129 | TGN38; TGN46; TGN48; | TGOLN2 | trans-golgi network protein 2 |
| | TGN51; TTGN2; MGC14722 | | |

The active TB group showed 5281 genes to be differentially expressed as compared to healthy controls, as compared to the latent group, which showed only differential expression of 3137 genes as compared to controls, possibly reflective of a more subdued, although clearly active immune response as shown by overexpression/representation of genes in the cytotoxic module. As an explanation, and not a limitation of the present invention, these results probably explain the observation that changes in additional modules were seen in active TB patients as compared to controls, but not in latent TB as compared to controls. These included overexpressed/represented genes in M1.2 (platelets, genes listed in Table 7A), and underexpressed/represented genes in M1.3 (B cells, genes listed in Table 7B), and M2.8 (T cells, genes listed in Table 7H), the latter perhaps being expected since in the T cells response to M. tuberculosis infection, it is possible that T cells are recruited to the site of infection and/or are suppressed during chronic infection. Genes in module M2.4, under-expressed/represented (genes listed in Table 7G) included transcripts encoding ribosomal protein family members whose expression is altered in acute infection and sepsis (Calvano, 2005; Thach, 2005), and genes in this module have also been shown to be underexpressed in SLE, liver transplant patients and those infected with Streptococcus (S). pneumoniae (Chaussabel, Immunity, 2005). The largest set of overexpressed genes (66 genes out of 90 detected, Table 71) in active TB was observed in module, M3.1, (IFN-inducible), and is in keeping with a role of IFN-γ in protection, however genes in this module were not differentially expressed in latent TB patients, who control the infection, as compared to controls. In active TB genes were underexpressed in a number of modules (M3.4, M3.6, M3.7, M3.8 and M3.9, genes listed in Tables 7L-7P) containing genes, which did not present a coherent functional module but consisted of an apparently diverse set of genes, and had also been observed to be underexpressed in liver transplant recipients (Chaussabel., 2008, Immunity).

Based on transcriptional analysis of whole blood and using this modular map approach active TB patients could be distinguished from latent TB patients. Furthermore, comparison of the modular map obtained for active TB in this study with other modular maps created for different diseases, it is clear that active TB patients have a distinct global transcriptional profile (FIG. 9), than observed in patients with SLE, transplant, melanoma or S. pneumoniae patients (Chaussabel, 2008, Immunity). Certain modules may be common to a number of diseases such as M2.4, included transcripts encoding ribosomal protein family members, which is underexpressed in active TB, SLE, liver transplant patients and those infected with S. pneumoniae. However, genes in other modules are less widely affected, such as M3.1 (IFN-inducible), which although overexpressed in active TB (FIG. 9) and SLE (Chaussabel, 2008, Immunity), but not other diseases, particularly S. pneumoniae, which shows no differential gene expression in M3.1 as compared to controls. Transcriptional profiles in SLE differ from active TB with respect to over or underexpession of genes in a number of other modules. Likewise, although overexpression of genes in modules M3.2 and M3.3 (“inflammatory”), M1.2 (platelets) and M1.5 (“myeloid”), and underexpression of genes in M3.4, 5, 6, 7, 8 and 9 (non-functionally coherent modules) is observed in active TB and S. pneumoniae these diseases can still be distinguished by this method since genes in modules M2.2 (neutrophils), M2.3 (erythrocytes), M3.5 (non-functionally coherent module) are overexpressed in S. pneumoniae as compared to controls but not differentially affected in active TB. Thus by retaining the complexity and magnitude of the data, yet organizing and reducing the dimension of the complex data, it is possible to distinguish different infectious and inflammatory diseases by transcriptional profiles of blood (Chaussabel, 2008, Immunity).

The present invention identifies a discreet differential and reciprocal dataset of transcriptional signatures in the blood of latent and active TB patients. Specifically, active TB patients showed an over-expression/representation of genes in functional IFN-inducible, inflammatory and myeloid modules, which on the other hand were down-regulated/under-represented in latent TB. Active TB patients showed and increased expression/over-representation of immunomodulatory genes PDL-1 and PDL-2, which may contribute to the immunopathogenesis in TB. Blood from latent TB patients showed an over-expression/representation of genes within a cytotoxic module, which may contribute to the protective response that contains the infection with M. tuberculosis in these patients and could provide biomarkers for testing efficacy of vaccinations in clinical trials. We believe the success of our preliminary study is achieved by the strict clinical criteria we have employed, accompanying immune reactivity studies to support attribution of latency, improved quality of RNA collection and isolation, advanced high throughput whole genome microarray platform, and sophisticated data mining tools to retain the magnitude of the gene expression but with an accessible format (Chaussabel et al., submitted). Such findings will be of value as diagnostics of latent and active TB, may yield insights into the potential mechanisms of immune protection (Latent TB) versus immune pathogenesis (Active TB), underlying these transcriptional differences, and the design of novel therapies for protection or in the design of immune therapeutics in active TB to achieve more rapid cure with anti-mycobacterial drugs.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof' as used herein refers to all permutations and combinations of the listed items preceding the term. For example, “A, B, C, or combinations thereof' is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Alizadeh, A. A., Eisen, M. B., Davis, R. E., Ma, C., Lossos, I. S., Rosenwald, A., Boldrick, J. C., Sabet, H., Tran, T., Yu, X., et al. (2000). Distinct types of diffuse large Bcell lymphoma identified by gene expression profiling. Nature 403, 503-511.

Allantaz, F., Chaussabel, D., Stichweh, D., Bennett, L., Allman, W., Mejias, A., Ardura, M., Chung, W., Wise, C., Palucka, K., et al. (2007). Blood leukocyte microarrays to diagnose systemic onset juvenile idiopathic arthritis and follow the response to IL-1 blockade. J Exp Med 204, 2131-2144.

Allantaz F, Chaussabel D, Banchereau J, Pascual V (2007) Microarray-based identification of novel biomarkers in IL-1-mediated diseases. Curr Opin Immunol 19: 623-632.

Baechler, E. C., Batliwalla, F. M., Karypis, G., Gaffney, P. M., Ortmann, W. A., Espe, K. J., Shark, K. B., Grande, W. J., Hughes, K. M., Kapur, V., et al. (2003). Interferon inducible gene expression signature in peripheral blood cells of patients with severe lupus. Proc Natl Acad Sci USA 100, 2610-2615.

Bennett, L., Palucka, A. K., Arce, E., Cantrell, V., Borvak, J., Banchereau, J., and Pascual, V. (2003). Interferon and granulopoiesis signatures in systemic lupus erythematosus blood. J Exp Med 197, 711-723.

Bittner, M., Meltzer, P., Chen, Y., Jiang, Y., Seftor, E., Hendrix, M., Radmacher, M., Simon, R., Yakhini, Z., Ben-Dor, A., et al. (2000). Molecular classification of cutaneous malignant melanoma by gene expression profiling. Nature 406, 536-540.

Bleharski, J. R., H. Li, C. Meinken, T. G. Graeber, M. T. Ochoa, M. Yamamura, A. Burdick, E. N. Sarno, M. Wagner, M. Rollinghoff, T. H. Rea, M. Colonna, S. Stenger, B. R. Bloom, D. Eisenberg, and R. L. Modlin. Use of genetic profiling in leprosy to discriminate clinical forms of the disease. Science (New York, N.Y. 2003. 301:1527-1530.

Burczynski, M. E., Twine, N. C., Dukart, G., Marshall, B., Hidalgo, M., Stadler, W. M., Logan, T., Dutcher, J., Hudes, G., Trepicchio, W. L., et al. (2005). Transcriptional profiles in peripheral blood mononuclear cells prognostic of clinical outcomes in patients with advanced renal cell carcinoma. Clin Cancer Res 11, 1181-1189.

Casanova, J. L., and L. Abel. Genetic dissection of immunity to mycobacteria: the human model. Annual review of immunology 2002. 20:581-620.

Chaussabel, D., Allman, W., Mejias, A., Chung, W., Bennett, L., Ramilo, O., Pascual, V., Palucka, A. K., and Banchereau, J. (2005). Analysis of significance patterns identifies ubiquitous and disease-specific gene-expression signatures in patient peripheral blood leukocytes. Ann N Y Acad Sci 1062, 146-154.

Chaussabel, C., Quinn, C., Shen, J., Patel, P, Glaser, C., Baldwin, N., Stichweh, D., Blankenship, D., Li, L., Munagala, I., Bennett, L., Allantaz, F., Mejias, A., Ardura, M., Kaizer, E., Monnet, L., Allman, W., Randall, H., Johnson, D., Lanier, A., Punar, M., Wittkowski, K. M., White, P., Fay, J., Klintmalm, G., Ramilo, O., Palucka, A. K., Banchereau, J., and Pascual, V. (2008). A Modular Framework for Biomarker and Knowledge Discovery from Blood Transcriptional Profiling Studies: Application to Systemic Lupus Erythematosus. Immunity. In press.

Cobb, J. P., Mindrinos, M. N., Miller-Graziano, C., Calvano, S. E., Baker, H. V., Xiao, W., Laudanski, K., Brownstein, B. H., Elson, C. M., Hayden, D. L., et al. (2005). Application of genome-wide expression analysis to human health and disease. Proc Natl Acad Sci USA 102, 4801-4806.

Gack, M. U., Y. C. Shin, C. H. Joo, T. Urano, C. Liang, L. Sun, O. Takeuchi, S. Akira, Z. Chen, S. Inoue, and J. U. Jung. TRIM25 RING-finger E3 ubiquitin ligase is essential for RIG-I-mediated antiviral activity. Nature 2007. 446:916-920.

Greenwald, R. J., Y. E. Latchman, and A. H. Sharpe. Negative co-receptors on lymphocytes. Current opinion in immunology 2002. 14:391-396.

Golub, T. R., Slonim, D. K., Tamayo, P., Huard, C., Gaasenbeek, M., Mesirov, J. P., Coller, H., Loh, M. L., Downing, J. R., Caligiuri, M. A., et al. (1999). Molecular classification of cancer: class discovery and class prediction by gene expression monitoring. Science 286, 531-537.

Jacobsen, M., J. Mattow, D. Repsilber, and S. H. Kaufmann. Novel strategies to identify biomarkers in tuberculosis. Biological chemistry 2008.

Jacobsen, M., D. Repsilber, A. Gutschmidt, A. Neher, K. Feldmann, H. J. Mollenkopf, A. Ziegler, and S. H. Kaufmann. Candidate biomarkers for discrimination between infection and disease caused by Mycobacterium tuberculosis. Journal of molecular medicine (Berlin, Germany) 2007. 85:613-621.

Kaizer, E. C., Glaser, C. L., Chaussabel, D., Banchereau, J., Pascual, V., and White, P. C. (2007). Gene expression in peripheral blood mononuclear cells from children with diabetes. J Clin Endocrinol Metab 92, 3705-3711.

Kaufmann, S. H., and A. J. McMichael. Annulling a dangerous liaison: vaccination strategies against AIDS and tuberculosis. Nature medicine 2005. 11:S33-44.

Keane, J. TNF-blocking agents and tuberculosis: new drugs illuminate an old topic. Rheumatology (Oxford, England) 2005. 44:714-720.

Li, X., B. Gold, C. O'Huigin, F. Diaz-Griffero, B. Song, Z. Si, Y. Li, W. Yuan, M. Stremlau, C. Mische, H. Javanbakht, M. Scally, C. Winkler, M. Dean, and J. Sodroski. Unique features of TRIM5alpha among closely related human TRIM family members. Virology 2007. 360:419-433.

Martinez, F. O., S. Gordon, M. Locati, and A. Mantovani. Transcriptional profiling of the human monocyte-to-macrophage differentiation and polarization: new molecules and patterns of gene expression. J Immunol 2006. 177:7303-7311.

Meroni, G., and G. Diez-Roux. TRIM/RBCC, a novel class of ‘single protein RING finger’ E3 ubiquitin ligases. Bioessays 2005. 27:1147-1157.

Mistry, R., J. M. Cliff, C. L. Clayton, N. Beyers, Y. S. Mohamed, P. A. Wilson, H. M. Dockrell, D. M. Wallace, P. D. van Helden, K. Duncan, and P. T. Lukey. Gene-expression patterns in whole blood identify subjects at risk for recurrent tuberculosis. The Journal of infectious diseases 2007. 195:357-365.

Nisole, S., J. P. Stoye, and A. Saib. TRIM family proteins: retroviral restriction and antiviral defence. Nat Rev Microbiol 2005. 3:799-808.

Pascual V, Allantaz F, Arce E, Punaro M, Banchereau J (2005) Role of interleukin-1 (IL-1) in the pathogenesis of systemic onset juvenile idiopathic arthritis and clinical response to IL-1 blockade. J Exp Med 201: 1479-1486.

Rajsbaum, R., J. P. Stoye, and A. O'Garra. Type I interferon-dependent and -independent expression of tripartite motif proteins in immune cells. European journal of immunology 2008. 38:619-630.

Ramilo, O., Allman, W., Chung, W., Mejias, A., Ardura, M., Glaser, C., Wittkowski, K. M., Piqueras, B., Banchereau, J., Palucka, A. K., and Chaussabel, D. (2007). Gene expression patterns in blood leukocytes discriminate patients with acute infections. Blood 109, 2066-2077.

Reljic, R. IFN-gamma therapy of tuberculosis and related infections. J Interferon Cytokine Res 2007. 27:353-364.

Reymond, A., G. Meroni, A. Fantozzi, G. Merla, S. Cairo, L. Luzi, D. Riganelli, E. Zanaria, S. Messali, S. Cainarca, A. Guffanti, S. Minucci, P. G. Pelicci, and A. Ballabio. The tripartite motif family identifies cell compartments. Embo J 2001. 20:2140-2151.

Rubins, K. H., L. E. Hensley, P. B. Jahrling, A. R. Whitney, T. W. Geisbert, J. W. Huggins, A. Owen, J. W. Leduc, P. O. Brown, and D. A. Relman. The host response to smallpox: analysis of the gene expression program in peripheral blood cells in a nonhuman primate model. Proceedings of the National Academy of Sciences of the United States of America 2004. 101:15190-15195.

Song, B., B. Gold, C. O'Huigin, H. Javanbakht, X. Li, M. Stremlau, C. Winkler, M. Dean, and J. Sodroski. The B30.2(SPRY) domain of the retroviral restriction factor TRIM5alpha exhibits lineage-specific length and sequence variation in primates. J Virol 2005. 79:6111-6121.

Thach, D. C., Agan, B. K., Olsen, C., Diao, J., Lin, B., Gomez, J., Jesse, M., Jenkins, M., Rowley, R., Hanson, E., et al. (2005). Surveillance of transcriptomes in basic military trainees with normal, febrile respiratory illness, and convalescent phenotypes. Genes Immun. 6(7): 588-95.

What is claimed is:

1. A method for distinguishing between active and latent Mycobacterium tuberculosis infection in a patient suspected of being infected with Mycobacterium tuberculosis, the method comprising:
obtaining a gene expression dataset from a whole blood sample from the patient;
determining the differential expression of one or more transcriptional gene expression modules that distinguish between infected patients and non-infected individuals, wherein the dataset demonstrates an aggregate change in the levels of polynucleotides in the one or more transcriptional gene expression modules as compared to matched non-infected individuals, and
distinguishing between active and latent Mycobacterium tuberculosis (TB) infection based on the one or more transcriptional gene expression modules that differentiate between active and latent infection.

2. The method of claim 1, further comprising the step of using the determined comparative gene product information to formulate a diagnosis.

3. The method of claim 1, further comprising the step of using the determined comparative gene product information to formulate a prognosis.

4. The method of claim 1, further comprising the step of using the determined comparative gene product information to formulate a treatment plan.

5. The method of claim 1, further comprising the step of distinguishing patients with latent TB from active TB patients.

6. The method of claim 1, wherein the module comprises a dataset of the genes in modules M1.2, M1.3, M1.4, M1.5, M1.8, M2.1, M2.4, M2.8, M3.1, M3.2, M3.3, M3.4, M3.6, M3.7, M3.8 or M3.9 to detect active pulmonary infection.

7. The method of claim 1, wherein the module comprises a dataset of the genes in modules M1.5, M2.1, M2.6, M2.10, M3.2 or M3.3 to detect a latent infection.

8. The method of claim 1, wherein the following genes are down-regulated in active pulmonary infection CD3, CTLA-4, CD28, ZAP-70, IL-7R, CD2, SLAM, CCR7 and GATA-3.

9. The method of claim 1, wherein the expression profile of FIG. 9 is indicative of active pulmonary infection.

10. The method of claim 1, wherein the expression profile of FIG. 10 is indicative of latent infection.

11. The method of claim 1, wherein the underexpression of genes in modules M3.4, M3.6, M3.7, M3.8 and M3.9 is indicative of active infection.

12. The method of claim 1, wherein the overexpression of genes in modules M3.1 is indicative of active infection.

13. The method of claim 1, further comprising the step of distinguishing TB infection from other bacterial infections by determining the gene expression in modules M2.2, M2.3 and M3.5, which are overexpressed by the peripheral blood mononuclear cells or whole blood in infection other than Mycobacterium.

14. The method of claim 1, further comprising the step of distinguishing the differential and reciprocal transcriptional signatures in the blood of latent and active TB patients using two or more of the following modules: M1.3, M1.4, M1.5, M1.8, M2.1, M2.4, M2.8, M3.1, M3.2, M3.3, M3.4, M3.6, M3.7, M3.8 or M3.9 for active pulmonary infection and modules M1.5, M2.1, M2.6, M2.10, M3.2 or M3.3 for a latent infection.

15. The method of claim 1, wherein the genes that are upregulated in active pulmonary TB infection versus a healthy patient are selected from Tables 7A, 7D, 71, 7J and 7K.

16. The method of claim 1, wherein the genes that are downregulated in active pulmonary TB infection versus a healthy patient are selected from Tables 7B, 7C, 7E, 7F, 7G, 7H, 7L, 7M, 7N, 7O and 7P.

17. The method of claim 1, wherein the genes that are upregulated in latent TB infection versus a healthy patient are selected from Table 8B.

18. The method of claim 1, wherein the genes that are downregulated in latent TB infection versus a healthy patient are selected from Tables 8A, 8C, 8D, 8E and 8F.

19. A method for distinguishing between active and latent Mycobacterium tuberculosis infection in a patient suspected of being infected with Mycobacterium tuberculosis, the method comprising:
obtaining a first gene expression dataset obtained from a first clinical group with active Mycobacterium tuberculosis infection, a second gene expression dataset obtained from a second clinical group with a latent Mycobacterium tuberculosis infection patient and a third gene expression dataset obtained from a clinical group of non-infected individuals;
generating a gene cluster dataset comprising the differential expression of genes between any two of the first, second and third datasets; and
determining a unique pattern of expression/representation that is indicative of latent infection, active infection or being healthy.

20. The method of claim 19, wherein each clinical group is separated into a unique pattern of expression/representation for each of the 119 genes of Table 6.

21. The method of claim 19, wherein values for the first and third datasets are compared and the values for the dataset from the third dataset are subtracted therefrom.

22. The method of claim 19, wherein values for the second and third datasets are compared and the values for the dataset from the third dataset are subtracted therefrom.

23. The method of claim 19, further comprising the step of comparing values for two different datasets and subtracting the values for the remaining dataset to distinguish between a patient with a latent infection, a patient with an active infection and a non-infected individual.

24. The method of claim 19, further comprising the step of using the determined comparative gene product information to formulate a diagnosis or a prognosis.

25. The method of claim 19, further comprising the step of using the determined comparative gene product information to formulate a treatment plan.

26. The method of claim 19, further comprising the step of distinguishing patients with latent TB from active TB patients.

27. The method of claim 19, further comprising of determining the expression levels of the genes: ST3GAL6, PAD14, TNFRSF12A, VAMP3, BR13, RGS19, PILRA, NCF1, LOC652616, PLAUR(CD87), SIGLEC5, B3GALT7, IBRDC3(NKLAM), ALOX5AP(FLAP), MMP9, ANPEP(APN), NALP12, CSF2RA, IL6R(CD126), RASGRP4, TNFSF14(CD258), NCF4, HK2, ARID3A, PGLYRP1(PGRP), which are underexpressed/underrepresented in the blood of Latent TB patients but not in the blood of Healthy individuals or Active TB patients.

28. The method of claim 19, further comprising of determining the expression levels of the genes: ABCG1, SREBF1, RBP7(CRBP4), C22orf5, FAM101B, S100P, LOC649377, UBTD1, PSTPIP-1, RENBP, PGM2, SULF2, FAM7A1, HOM-TES-103, NDUFAF1, CES1, CYP27A1, FLJ33641, GPR177, MID1IP1(MIG-12), PSD4, SF3A1, NOV(CCN3), SGK(SGK1), CDK5R1, LOC642035, which are overexpressed/overrepresented in the blood of Healthy control individuals but were underexpressed/underrepresented in the blood of Latent TB patients, and underexpressed/underrepresented in the blood of Active TB patients.

29. The method of claim 19, further comprising of determining the expression levels of the genes: ARSG, LOC284757, MDM4, CRNKL1, IL8, LOC389541, CD300LB, NIN, PHKG2, HIP1, which are overexpressed/overrepresented in the blood of Healthy individuals, are underexpressed/underrepresented in the blood of both Latent and Active TB patients.

30. The method of claim 19, further comprising of determining the expression levels of the genes: PSMB8(LMP7), APOL6, GBP2, GBP5, GBP4, ATF3, GCH1, VAMPS, WARS, LIMK1, NPC2, IL-15, LMTK2, STX11(FHL4), which are overexpressed/overrepresented in the blood of Active TB, and underexpressed/underrepresented in the blood of Latent TB patients and Healthy control individuals.

31. The method of claim 19, further comprising of determining the expression levels of the genes: FLJ11259(DRAM), JAK2, GSDMDC1(DF5L)(FKSG10), SIPAIL1, [2680400](KIAA1632), ACTA2(ACTSA), KCNMB1(SLO-BETA), which are overexpressed/overrepresented in blood from Active TB patients, and underexpressed/underrepresented in the blood from Latent TB patients and Healthy control individuals.

32. The method of claim 19, further comprising of determining the expression levels of the genes: SPTANI, KIAAD179(Nnp1)(RRP1), FAM84B(NSE2), SELM, IL27RA, MRPS34, [6940246](IL23A), PRKCA(PKCA), CCDC41, CD52(CDW52), [3890241](ZN404), MCCC1(MCCA/B), SOX8, SYNJ2, FLJ21127, FHIT, which are underexpressed/underrepresented in the blood of Active TB patients but not in the blood of Latent TB patients or Healthy Control individuals.

33. The method of claim 19, further comprising of determining the expression levels of the genes: CDKL1(p42), MICALCL, MBNL3, RHD, ST7(RAY1), PPR3R1, [360739](PIP5K2A), AMFR, FLJ22471, CRAT(CAT1), PLA2G4C, ACOT7(ACT)(ACH1), RNF182, KLRC3(NKG2E), HLA-DPB1, which are underexpressed/underrepresented in the blood of Healthy Control individuals, overexpressed/overrepresented in the blood of the Latent TB patients, and overexpressed/overrepresented in the blood of Active TB patients.

34. A method for distinguishing between active and latent mycobacterium tuberculosis infection in a patient suspected of being infected with Mycobacterium tuberculosis, the method comprising:
obtaining a gene expression dataset from a whole blood sample;
sorting the gene expression dataset into one or more transcriptional gene expression modules; and
mapping the differential expression of the one or more transcriptional gene expression modules that distinguish between active and latent Mycobacterium tuberculosis infection, thereby distinguishing between active and latent Mycobacterium tuberculosis infection.

35. The method of claim 34, wherein the dataset comprises TRIM genes.

36. The method of claim 34, wherein the dataset comprises TRIM genes, and TRIM 5, 6, 19(PML), 21, 22, 25, 68 are overrepresented/expressed in active pulmonary TB.

37. The method of claim 34, wherein the dataset comprises TRIM genes, and TRIM 28, 32, 51, 52, 68, are underepresented/expressed in active pulmonary TB.

38. A method of diagnosing a patient with active and latent Mycobacterium tuberculosis infection in a patient suspected of being infected with mycobacterium tuberculosis, the method comprising detecting differential expression of one or more transcriptional gene expression modules that distinguish between infected and non-infected patients obtained from whole blood, wherein whole blood demonstrates an aggregate change in the levels of polynucleotides in the one or more transcriptional gene expression modules as compared to matched non-infected patients, thereby distinguishing between active and latent mycobacterium tuberculosis infection.

39. The method of claim 38, further comprising the step of using the determined comparative gene product information to formulate a diagnosis.

40. The method of claim 38, further comprising the step of using the determined comparative gene product information to formulate a prognosis.

41. The method of claim 38, further comprising the step of using the determined comparative gene product information to formulate a treatment plan.

42. The method of claim 38, wherein the module comprises a dataset of the genes in modules M1.2, M1.3, M1.4, M1.5, M1.8, M2.1, M2.4, M2.8, M3.1, M3.2, M3.3, M3.4, M3.6, M3.7, M3.8, or M_{3.9} to detect active pulmonary infection.

43. The method of claim 38, wherein the module comprises a dataset of the genes in modules M1.5, M2.1, M2.6, M2.10, M3.2 or M3.3 to detect a latent infection.

44. The method of claim 38, wherein the following genes are down-regulated in active pulmonary infection CD3, CTLA-4, CD28, ZAP-70, IL-7R, CD2, SLAM, CCR7 and GATA-3.

45. The method of claim 38, wherein the expression profile of modules of FIG. 9 is diagnostic of active pulmonary infection.

46. The method of claim 38, wherein the expression profile of modules of FIG. 10 is diagnostic of latent infection.

47. The method of claim 38, wherein the underexpression of genes in modules M3.4, M3.6, M3.7, M3.8 and M3.9 is indicative of active infection.

48. The method of claim 38, wherein the overexpression of genes in modules M3.1 is indicative of active infection.

49. The method of claim 38, further comprising the step of distinguishing TB infection from other bacterial infections by determining the gene expression in modules M2.2, M2.3 and M3.5, which are overexpressed by the peripheral blood mononuclear cells or whole blood in infection other than Mycobacterium.

50. The method of claim 38, further comprising the step of distinguishing the differential and reciprocal transcriptional signatures in the blood of latent and active TB patients using two or more of the following modules: M1.3, M1.4, M1.5, M1.8, M2.1, M2.4, M2.8, M3.1, M3.2, M3.3, M3.4, M3.6, M3.7, M3.8 or M3.9 for active pulmonary infection and modules M1.5, M2.1, M2.6, M2.10, M3.2 or M3.3 for a latent infection.

51. A kit for diagnosing a patient with active and latent mycobacterium tuberculosis infection in a patient suspected of being infected with Mycobacterium tuberculosis, the kit comprising:
a gene expression detector for obtaining a gene expression dataset from the patient; and
a processor capable of comparing the gene expression to pre-defined gene module dataset that distinguish between infected and non-infected patients obtained from whole blood, wherein whole blood demonstrates an aggregate change in the levels of polynucleotides in the one or more transcriptional gene expression modules as compared to matched non-infected patients, thereby distinguishing between active and latent Mycobacterium tuberculosis infection.

52. A system of diagnosing a patient with active and latent Mycobacterium tuberculosis infection comprising:
a gene expression dataset from the patient; and
a processor capable of comparing the gene expression to pre-defined gene module dataset that distinguish between infected and non-infected patients obtained from whole blood, wherein whole blood demonstrates an aggregate change in the levels of polynucleotides in the one or more transcriptional gene expression modules as compared to matched non-infected patients, thereby distinguishing between active and latent Mycobacterium tuberculosis infection, wherein the modules are selected from M1.3, M1.4, M1.5, M1.8, M2.1, M2.4, M2.8, M3.1, M3.2, M3.3, M3.4, M3.6, M3.7, M3.8 or M3.9 for active pulmonary infection and modules M1.5, M2.1, M2.6, M2.10, M3.2 or M3.3 for a latent infection.
